(19) United States
(12) Patent Application Publication
(10) Pub. No.: US 2025/0057463 A1
(43) Pub. Date: Feb. 20, 2025

(54) ASSESSING CARDIAC RISK

(71) Applicant: ZOLL Medical Corporation, Chelmsford, MA (US)

(72) Inventors: Jeffery T. Mitchell, Mars, PA (US); Francesco Nicolo, Oakmont, PA (US); Michael A. Sipe, Pittsburgh, PA (US); Nan Song, Pittsburgh, PA (US)

(21) Appl. No.: 18/805,891

(22) Filed: Aug. 15, 2024

Related U.S. Application Data
(60) Provisional application No. 63/520,042, filed on Aug. 16, 2023.

(51) Int. Cl. A61B 5/352; A61B 5/00; A61B 5/282; A61B 5/355; A61B 5/363; A61B 5/366

(52) U.S. Cl. CPC A61B 5/352; A61B 5/282; A61B 5/355; A61B 5/363; A61B 5/366; A61B 5/6802; A61B 5/7267; A61B 5/7275

(57) ABSTRACT

Disclosed are systems, methods, and computer program product for cardiac event risk assessment including for assessing future cardiac risk for a patient. The disclosed systems can include a wearable cardiac sensing device configured to be removably, bodily-attached to the patient, configured to sense signals from a patient and provide the digitized to a processor configured to determine cardiac event risk assessment. In some embodiments, cardiac event risk assessment can be determined based on a cardiovibration classifier, counts or burdens associated with premature ventricular contractions (PVC) patterns, and multi-focal PVCs.

### CROSS-REFERENCE TO RELATED APPLICATIONS

This application claims priority under 35 USC § 119 (e) to U.S. Patent Application Ser. No. 63/520,042, filed on Aug. 16, 2023, the entire contents of which are hereby incorporated by reference.

### BACKGROUND

There are a wide variety of electronic and mechanical devices for monitoring and treating patients' medical conditions. In some examples, depending on the underlying medical condition being monitored or treated, medical devices such as cardiac monitors or defibrillators may be surgically implanted or externally connected to the patient. In some cases, a patient with a known cardiac condition may be provided with a device that monitors the patient's cardiac activity. The device or a remote server in communication with the device may analyze the patient's cardiac activity as part of monitoring the patient's ongoing cardiac health.

While existing systems may be configured to monitor a patient's ongoing health, they are often unable to provide a prediction of a patient's risk of having a cardiac event in the future such as a sudden cardiac arrest. During a sudden cardiac arrest there is a complete stoppage of all heart activity. A patient may experience a sudden, unexpected loss of heart function, breathing, and consciousness that requires immediate medical intervention (e.g., CPR or defibrillator).

### SUMMARY

In some implementations a cardiac event risk assessment system for assessing future cardiac risk for a patient includes a wearable cardiac sensing device configured to be removably, bodily-attached to the patient, the wearable cardiac sensing device configured to sense cardiovibration signals from a patient and provide digitized cardiovibration signals, and a processor in communication with the wearable cardiac sensing device. The processor may be further configured for identifying cardiovibration data points corresponding to a predetermined physiological marker of the patient based on the digitized cardiovibration signal, analyzing a frequency spectrum corresponding to the identified cardiovibration data points to determine one or more cardiovibration frequency metrics for the patient associated with the predetermined physiological marker of the patient, providing the one or more cardiovibration frequency metrics for the patient to a trained cardiovibration classifier, wherein the trained cardiovibration classifier is trained at least in part on historical cardiovibration frequency metrics associated with the predetermined physiological marker derived from a plurality of patients, optimizing the trained cardiovibration classifier based on one or more predetermined classifier evaluation metrics, and outputting, based on the trained and optimized cardiovibration classifier, risk information concerning a future cardiac ischemia and/or a sudden cardiac arrest event occurring within a predetermined future period of time.

In some implementations a cardiac event risk assessment system for assessing future cardiac risk for a patient includes a wearable cardiac sensing device configured to be removably, bodily-attached to the patient, and configured to sense electrocardiogram (ECG) signals from a patient and provide digitized ECG signals and a processor in communication with the wearable cardiac sensing device. The processor may be further configured for identifying ECG data points corresponding to premature ventricular contractions (PVCs) of the patient based on the digitized ECG signal, determining one or more PVC patterns based on the identified ECG data points corresponding to the PVCs of the patient, determining one or more of a count or a burden associated with the one or more PVC patterns, and outputting risk information concerning sudden cardiac arrest event occurring within a predetermined future period of time by applying a trained machine learning algorithm to the determined one or more of the count or the burden associated with the patient, wherein the trained machine learning algorithm is trained at least in part on historical PVC pattern data derived from a plurality of patients.

In some implementations a cardiac event risk assessment system for assessing future cardiac risk for a patient includes a wearable cardiac sensing device configured to be removably, bodily-attached to the patient, and configured to sense electrocardiogram (ECG) signals from a patient and provide digitized ECG signals and a processor in communication with the wearable cardiac sensing device. The processor may be further configured for identifying ECG data points corresponding to premature ventricular contractions (PVCs) of the patient based on the digitized ECG signal, determining multi-focal PVC data from the identified ECG data points corresponding to PVCs by applying an unsupervised machine learning algorithm, wherein the unsupervised machine learning algorithm is trained to determine_multiple focal points within a set of PVCs, and generating risk information for the patient indicative of a future sudden cardiac arrest event by applying a trained classifier to the determined multi-focal PVC data, wherein the classifier is trained on a historical data set of multi-focal PVC data derived from a plurality of patients. Optionally, the processor may be configured for identifying QRS notch data points corresponding to QRS notches of the patient based on the digitized ECG signal, where generating risk information for the patient includes applying a second classifier trained on a historical data set of QRS notch data derived from a plurality of patients.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example wearable cardiac sensing system including a wearable cardiac sensing device.

FIG. 2 depicts an example adhesive patch of a wearable cardiac sensing device.

FIG. 3 depicts an example cardiac sensing unit of a wearable cardiac sensing device.

FIG. 4 depicts an example of using an adhesive patch with a cardiac sensing unit.

FIG. 5 depicts an example electronic architecture for a cardiac sensing unit.

FIG. 6 depicts another example wearable cardiac sensing system including a wearable cardiac sensing device.

FIG. 7 depicts an example garment-based monitoring device of a wearable cardiac sensing device.

FIG. 8 depicts an example electronic architecture for a garment-based monitoring device.

FIG. 9 depicts an example process flow for applying an improved cardiovibration analysis for determining a risk of future cardiac events.

FIG. 10 depicts a sample set of data points corresponding to cardiovibration data.

FIG. 11 depicts a sample set of data points corresponding to cardiovibration data in the power spectrum.

FIG. 12 depicts an example of a cardiovibration frequency metric.

FIG. 13 depicts a second example of a cardiovibration frequency metric.

FIG. 14 depicts a third example of a cardiovibration frequency metric.

FIG. 15 depicts an example process flow for a clinician using an improved cardiovibration analysis for determining a risk of a future cardiac event in a patient.

FIG. 16 depicts an example of a graphic provided to a clinician using an improved cardiovibration analysis for determining a risk of a future cardiac event in a patient.

FIG. 17 depicts an example process flow for using PVC pattern analysis for determining a risk of future cardiac events in a patient.

FIG. 18A depicts a sample set of data points corresponding to a bigeminy PVC pattern.

FIG. 18B depicts a sample set of data points corresponding to a quadrageminy PVC pattern.

FIG. 18C depicts a sample set of data points corresponding to a couplet PVC pattern.

FIG. 18D depicts a sample set of data points corresponding to a triplet PVC pattern.

FIG. 18E depicts a sample set of data points corresponding to multi-focal PVCs.

FIG. 18F depicts a sample set of data points corresponding to PVC pattern data.

FIG. 19A depicts training data for a particular PVC pattern (e.g., isolated PVCs) in patients experiencing a cardiac event and patients not experiencing a cardiac event.

FIG. 19B depicts training data for a particular PVC pattern (e.g., PVC doublets) in patients experiencing a cardiac event and patients not experiencing a cardiac event.

FIG. 19C depicts training data for a particular PVC pattern (e.g., PVC triplets) in patients experiencing a cardiac event and patients not experiencing a cardiac event.

FIG. 19D depicts training data a for a particular PVC pattern (e.g., PVC quadruplets) in patients experiencing a cardiac event and patients not experiencing a cardiac event.

FIG. 19E depicts training data for a particular PVC pattern (e.g., NSVT) in patients experiencing a cardiac event and patients not experiencing a cardiac event.

FIG. 19F depicts training data for a particular PVC pattern (e.g., bigeminy) in patients experiencing a cardiac event and patients not experiencing a cardiac event.

FIG. 19G depicts training data for a particular PVC pattern (e.g., trigeminy) in patients experiencing a cardiac event and patients not experiencing a cardiac event.

FIG. 19H depicts training data for a particular PVC pattern (e.g., quadrageminy) in patients experiencing a cardiac event and patients not experiencing a cardiac event.

FIG. 19I depicts training data for a particular PVC pattern (e.g., 5-geminy) in patients experiencing a cardiac event and patients not experiencing a cardiac event.

FIG. 20 depicts an example process flow for using multi-focal PVC analysis in determining a risk of future cardiac events in a patient.

FIG. 21 depicts a sample set of data points corresponding to QRS data from a patient with multi-focal PVCs.

FIG. 22A depicts a histogram of morphology scores for PVCs across a patient.

FIG. 22B depicts a histogram of patient PVC characteristics data (e.g., R-R duration).

FIG. 22C depicts a scatterplot of patient PVC characteristics data (e.g., current beat R-R duration and previous beat R-R duration).

FIG. 22D depicts a histogram of patient PVC characteristics data (e.g., prematurity index).

FIG. 22E depicts a histogram of patient PVC characteristics data (e.g., QRS duration).

FIG. 22F depicts a histogram of patient PVC characteristics data (e.g., morphology score).

FIG. 22G depicts a histogram of patient PVC characteristics data (e.g., R-S segment area).

FIG. 22H depicts a histogram of patient PVC characteristics data (e.g., Q-R/R-S segment area).

FIG. 23 depicts clustering algorithms applied to PVC characteristics data.

FIG. 24 depicts validation data for classifiers applied to PVC data.

FIG. 25 depicts PVC parameters for multi-focal PVC data.

FIG. 26 depicts an example process flow for using QRS notch analysis in determining a risk of future cardiac events in a patient.

FIG. 27 depicts QRS data including QRS notches.

FIG. 28 depicts QRS notch analysis performance in data including normal beats and PVC beats.

FIG. 29A depicts a graph of example estimation of risk scores.

FIG. 29B depicts a second graph of example estimation of risk scores.

### DETAILED DESCRIPTION

The present disclosure relates to a wearable cardiac event risk assessment system that is configured to assess future cardiac risk for a patient. In some embodiments, the cardiac event risk assessment system may be configured to assess the future risk of having a sudden cardiac arrest. In some embodiments, the cardiac event risk assessment may be configured to assess the future risk of having cardiac ischemia. In some scenarios, ischemia can be a precursor to sudden cardiac arrest, and is characterized by a lack of blood flow and oxygen to the heart muscle that may cause damage to the tissues in the heart muscle. Embodiments of the wearable cardiac event risk assessment system may analyze frequencies associated with cardiovibration data points to determine risk of a future cardiac ischemia event and/or a future sudden cardiac arrest event. Embodiments of the wearable cardiac event risk assessment system may also apply one or more trained machine learning algorithms to data associated with premature ventricular contractions (PVCs) indicative of counts, burdens, and/or patterns to determine the risk of a future sudden cardiac arrest event. Embodiments of the wearable cardiac event risk assessment system may also apply one or more machine learning algorithms to data associated with PVCs and QRS patterns to determine the risk of a future sudden cardiac arrest event. For example, the risk associated with a future adverse cardiac event can be presented to interested parties, such as a physician, caregiver, and/or the patient themselves, in the form of a risk estimate valid for multiple future time periods, e.g., the next hour, the next three hours, the next 6 hours, the next 24 hours, the next three days, the next week, the next 2 weeks, the next month, the next 3 months, the next 6 months, and/or other configurable duration. For example, the interested party may select a certain duration via a user manipulable parameter for which to learn about the patient's associated risk. In implementations, the associated risk estimates for the various future time periods can include information reflecting a confidence or other reliability measure for the associated risk estimate, e.g., in the form of a confidence rating or other such metric.

Cardiac event risk assessment systems implementing the devices, methods, and techniques disclosed herein can be used to provide assessments regarding the risk of future cardiac events taking place in patients. For example, patients with known or suspected cardiac conditions may be prescribed a wearable cardiac monitor or a wearable cardiac monitor and treatment device that the patient must wear for the prescribed period of time. During the period of wear, the cardiac monitor may generate cardiovibration signals and ECG signals for the patient and may collect other physiological data about the patient, such as data regarding the patient's movement, posture, lung fluid levels, respiration rate, and/or the like. In implementations, the cardiac monitor can estimate initial and/or baseline values for these physiological parameters at an initial period of time during the beginning of the patient's use of the device. For instance, baseline ECG, cardiovibration, and/or lung fluid level recordings may be taken within the first hour, first two hours, first three hours, first day, first week, or other predetermined initial period of time. In implementations, such baseline information can be used to establish a baseline risk estimate for future cardiac events for the patient in a manner described in further detail below. Thereafter, the risk estimates can vary during the patient's use of the device. In such cases, the implementations herein can present such changes to the risk estimates with information showing the changes relative to the baseline values.

The ECG signals and cardiovibration signals may be analyzed, at the cardiac monitor and/or at a remote server in communication with the cardiac monitor, to determine if the patient is at risk of having a future cardiac event. Example cardiac events include cardiac ischemia, or sudden cardiac arrest (SCA). In examples, potential cardiac ischemia and/or sudden cardiac arrest events are based on how such events are labeled in the training data associated with the classifiers in accordance with implementations herein. For instance, SCA events can include developing treatable ventricular tachycardia (VT) or treatable ventricular fibrillation (VF) event that is usually a precursor to SCA. As such, the training data can include such events in determining SCA risk.

In some implementations, a cardiac event risk assessment system as described herein is integrated into a system for cardiac event monitoring, which is configured to monitor for the occurrence of cardiac conditions such as arrhythmias including ventricular ectopic beats (VEB), ventricular runs/ventricular tachycardia, bigeminy, supraventricular ectopic beats (SVEB), supraventricular tachycardia, atrial fibrillation, ventricular fibrillation, pauses, 2^{nd} AV blocks, 3^{rd} AV blocks, bradycardia, and/or other types of tachycardia. For example, such a system may be used for both monitoring of the occurrence of cardiac conditions as well as for providing an assessment of future cardiac event risk. An assessment of future cardiac event risk can be provided irrespective of whether the occurrence of cardiac conditions is monitored. In some implementations, a cardiac event risk assessment system as described herein is integrated into a system for treating certain cardiac arrhythmic events, such as a wearable cardioverter defibrillator system as described in further detail below that is configured to monitor for the occurrence of treatable cardiac arrhythmias, including VT, VF, bradycardia, tachycardia and issue one or more therapeutic electrical pulses (e.g., pacing pulses, cardioversion pulses, and/or defibrillation pulses) to the patient.

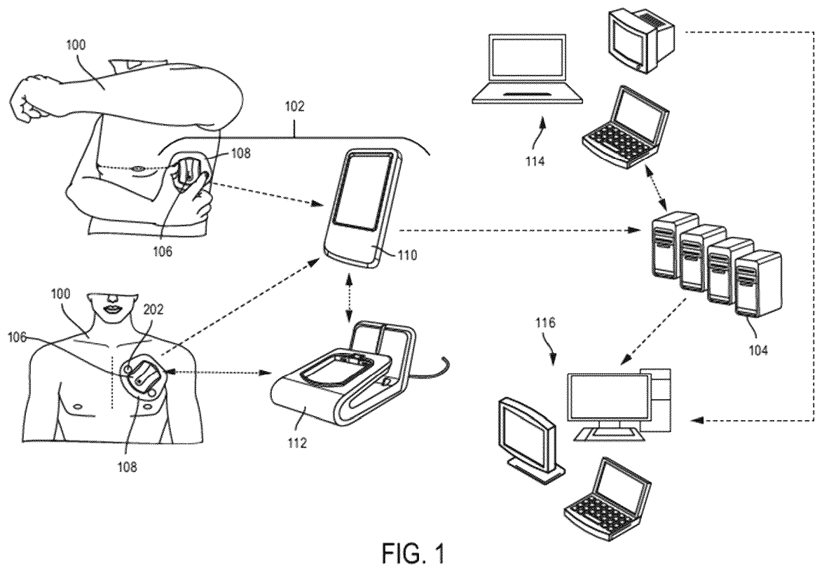

For example, a cardiac event risk assessment system including a cardiac monitor is shown in FIG. 1. The cardiac event risk assessment system includes a remote server 102 in network communication with a patient-worn mounted cardiac monitor 106 For example, the communication can be based on a predetermined radio wave wireless communications standard based on one or more of the IEEE 802.11x Wi-Fi protocol, the Bluetooth protocol, the ZigBee standard, RFID standards, ultra wide band standards, or WiMAX standard. The system (e.g., either on the remote server 102 or within the monitor 106) is configured to perform processing or analyses of the ECG signal received from the cardiac monitor 106, such as band pass filtering, detecting R-R intervals, detecting QRS intervals, and/or heart rate estimation. The system (e.g., either on the remote server 102 or within the cardiac monitor 106) also perform other processing or analysis of one or more cardiovibration signals obtained from the patient. Additionally or alternatively, a patient with a known cardiac condition is prescribed a wearable cardiac monitor configured to provide the patient and/or clinical provider with an assessment of future cardiac events. Additionally or alternatively, the cardiac monitor is configured to provide a treatment, such as therapeutic defibrillation, cardioversion, anti-tachycardiac pacing, and/or anti-bradycardiac pacing pulses, upon detecting certain types of treatable arrhythmias (e.g., ventricular fibrillation, ventricular tachycardia, bradycardia, etc.). The patient may similarly use the wearable cardiac monitor and treatment device for the prescribed period of time, while the device and/or remote server analyzes the patient's ECG signals and/or cardiovibrations to determine future cardiac event risk the patient.

As discussed in further detail below, the cardiac event risk assessment system implementing the devices, methods, and techniques disclosed herein provides improved methods of analyzing ECG signals and/or cardiovibrations to determine risk of future cardiac events including, for example, cardiac ischemia and/or sudden cardiac arrest. More specifically, in some examples described below, the cardiac event risk assessment system implementing the devices, methods, and techniques disclosed herein identifies cardiovibration data points corresponding to a predetermined physiological marker (e.g., S1, S2 sounds), and analyzes a frequency spectrum corresponding to the identified cardiovibration data points to determine cardiovibration frequency metrics. The cardiovibration frequency metrics may then be provided to a trained cardiovibration classifier that outputs risk information concerning a future cardiac ischemic event and/or sudden cardiac arrest.

Additionally or alternatively, the cardiac event risk assessment system implementing the devices, methods, and techniques disclosed herein provides improved methods for analyzing ECG data to determine risk of future cardiac events including sudden cardiac arrest. More specifically, the cardiac event risk assessment system implementing the devices, methods, and techniques disclosed herein determines one or more PVC patterns based on ECG data, determines counts and/or burdens associated with the determined PVC patterns, and outputs risk information concerning sudden cardiac assessment by applying a trained machine learning algorithm to the determined one or more of the count or the burden associated with the patient. For example, the trained machine learning algorithm may be trained at least in part on historical PVC pattern data derived from a plurality of patients.

Additionally or alternatively, the cardiac event risk assessment system implementing the devices, methods, and techniques disclosed herein determines multi-focal PVC data from the identified ECG data points corresponding to PVCs by applying an unsupervised machine learning algorithm and generating risk information for the patient indicative of a future sudden cardiac arrest event by applying a trained classifier to the determined multi-focal PVC data. Optionally, the cardiac event risk assessment system may include a second classifier trained on QRS notch data that contributes to the generation of risk information for the patient indicative of a future sudden cardiac arrest event.

In implementations, the cardiac event risk assessment system includes a wearable cardiac sensing device configured to be bodily-attached to the patient. Examples of wearable cardiac sensing devices may include an adhesive device configured to be adhered to the skin of the patient or a garment-based device configured to be worn under the patient's clothing. The wearable cardiac sensing device includes a number of physiological sensors, including one or more ECG electrodes configured to sense electrical signals from a skin surface of the patient. The wearable cardiac sensing device also includes an ECG digitizing circuit configured to provide digitized signals of the patient based on the sensed electrical signals that are stored in a non-transitory memory of the wearable cardiac sensing device. Other physiological sensors of the wearable cardiac sensing device may include, for instance, a motion sensor such as an single-axial accelerometer, bi-axial accelerometer, tri-axial accelerometer, and/or multi-channel accelerometer and/or a gyroscope configured to monitor for body movement and/or body position of the patient, a respiration sensor, e.g., an accelerometer or biovibrational sensor configured to monitor low frequency chest wall motion, a biovibrational sensor (e.g., configured to monitor for vibrational signals from within the body of the patient such as cardiovibrations, lung vibrations, etc.), a blood pressure sensor, a temperature sensor, a pressure sensor, a humidity sensor, a P-wave optimized ECG sensor (e.g., an ECG sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), a lung fluid monitor (e.g., implemented through radio-frequency based sensors configured to transmit radio-frequency microwaves through a chest cavity of the patient and receive reflections to monitor for lung fluid changes over a period of time) and so on. In some implementations, the wearable cardiac sensing device also include a circuit configured to provide digitized signals of the vibrations signals detected from the patient's heart (e.g., cardiovibrations).

The cardiac event risk assessment system also includes a processor in communication with the wearable cardiac sensing device. The processor may be located at the wearable cardiac sensing device or at a remote server in communication with the wearable cardiac sensing device. Alternatively, the functions of the processor may be split between the wearable cardiac sensing device and the remote server. The processor is configured to extract a predetermined timeseries of ECG data points from the digitized ECG signals and identify ECG feature data points corresponding to a certain ECG feature from within the digitized ECG signals of the patient. For example, the certain ECG feature may be a QRS complex or a feature considered a QRS complex candidate that needs to be confirmed as a normal QRS complex or corresponding to an abnormal cardiac beat, such as a premature ventricular contraction.

The processor may also be configured to identify cardiovibration data points corresponding to a predetermined physiological marker (e.g., S1, S2 sounds) of the patient based on a digitized cardiovibration signal.

The processor may be further configured to apply frequency analysis and/or machine learning based techniques on the digitized ECG signals, digitized cardiovibration signals, or data derived therefrom, to generate assessments on the risk of future cardiac events.

In one example use case, a cardiologist may prescribe that a patient with a suspected cardiac condition use a wearable cardiac sensing device for a prescribed time period. The wearable cardiac sensing device may include an adhesive patch, a cardiac sensing unit configured to be mounted onto the adhesive patch, and a portable gateway in communication with the cardiac sensing unit and with a remote server. The patient may use the wearable cardiac sensing device during their daily activities (e.g., with the adhesive patch and cardiac sensing unit worn under the patient's clothes and the portable gateway carried with the patient) during the prescribed time period. As the wearable cardiac sensing device is being used by the patient, the wearable cardiac sensing device may generate provides ECG signals for the patient based on sensed electrical activity. At the portable gateway and/or at the remote server, the ECG signals may be subjected to analysis. For example, the portable gateway and/or the remote server may analyze ECG signals to generate or obtain data regarding abnormal cardiac events or cardiovibration signals such as PVCs or notched QRS complexes. In one implementation, the gateway and/or the remote server may determine whether the patient is at risk for future cardiac events such as cardiac ischemia and/or sudden cardiac arrest based on the analysis of ECG signals and/or cardiac vibration signals. In implementations, the determination whether the patient is at risk for future cardiac events can be carried out on both the gateway and the remote server. In implementations, some portion of the determination whether the patient is at risk for future cardiac events can be carried on the gateway, and another portion of such determination can be carried out at the remote server. In implementations, an initial determination whether the patient is at risk of future cardiac events can be carried on the gateway, and a confirmation, e.g., verification of the initial determined can be carried out at the remote server. In some or all such implementations, the remote server may then can prepare reports for the patient's cardiologist summarizing the patient's risk for future cardiac events. For example, such reports that may can be displayed on a cardiac dashboard displayed via a web-based portal with access to the remote server presenting information on a number of patients being overseen by the cardiologist or otherwise transmitted to the cardiologist. For instance, the remote server may send electronic email (e-mail) reports summarizing on the risk of a patient having a cardiac event to the patient's cardiologist.

The cardiac event risk assessment system described herein may provide advantages over prior art systems. For example, cardiovibration frequencies associated with physiological markers can be analyzed to determine if a patient has an elevated risk of having a future cardiac event. Similarly, ECG data can be analyzed to determine QRS signals, PVC data and the like. Additionally, artificial intelligence based systems can be applied to the ECG based data in order to determine whether a patient has an elevated risk of having a future cardiac event. By more accurately identifying patients with elevated risks of having a future cardiac event, clinicians can more effectively provide treatment options, which may help patients experience better long-term health outcomes.

FIG. 1 illustrates an example of a cardiac event risk assessment system for applying machine learning based systems to ECG signals and/or cardiovibration signals gathered from a patient 100, according to implementations disclosed herein.

The cardiac event risk assessment system of FIG. 1 includes a wearable cardiac sensing device 102 configured for to be bodily-attached to the patient 100 (e.g., for long-term continuous wear by the patient 100), where the wearable cardiac sensing device 102 is in communication with a remote server 104. The wearable cardiac sensing device 102 includes a number of physiological sensors. As an example, the physiological sensors may include one or more ECG electrodes configured to sense electrical signals from a skin surface of the patient 100. In another example, the physiological sensors may include one or more sensors configured to obtain cardiovibration signals from the cardiac system including cardiovibration sensors.

In the example of FIG. 1, the wearable cardiac sensing device 102 includes a cardiac sensing unit 106 removably coupled to a removable adhesive patch 108. However, other implementations of the wearable cardiac event risk assessment system may include different embodiments of a wearable cardiac sensing device 102, as described in further detail below.

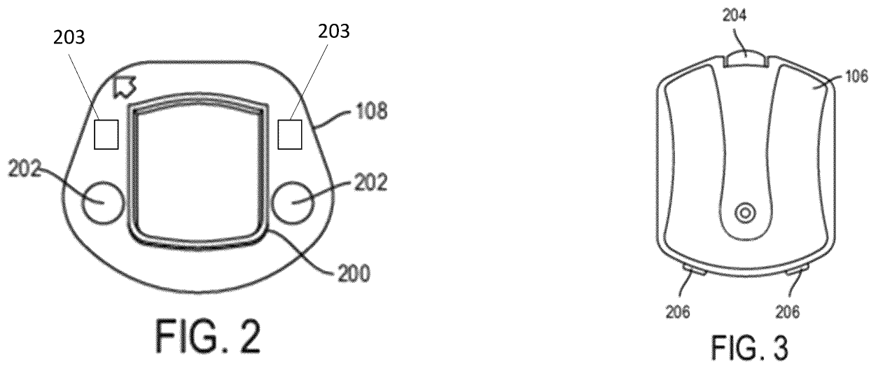

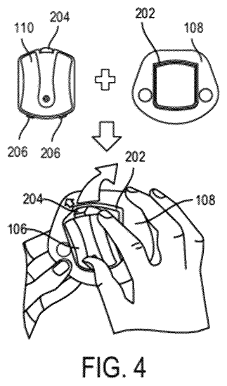

The adhesive patch 108 is configured to be adhesively attached or coupled to the skin of the patient 100. The adhesive patch 108 is further configured such that the cardiac sensing unit 106 may be removably attached to the adhesive patch 108. For example, referring to FIG. 2, the adhesive patch 108 may include a frame 200 (e.g., a plastic frame) delineating the boundary of the region of the adhesive patch 108 that is configured for receiving the cardiac sensing unit 106. The adhesive patch 108 may be disposable (e.g., single-, few-, or multiple-use patches) and may be made of biocompatible, non-woven material. In some implementations, the cardiac sensing unit 106 may be designed for long-term, continuous usage. In such implementations, the connection between the cardiac sensing unit 106 and the adhesive patch 108 may be configured to be reversible. For example, the cardiac sensing unit 106 may be configured to be removably attached to the adhesive patch 108. In implementations, as shown in FIG. 3, the cardiac sensing unit 106 may include components such as a snap-in clip 204 that is configured to secure the cardiac sensing unit 106 to the adhesive patch 108 upon attachment to the patch frame 200. After the cardiac sensing unit 106 is attached to the frame 200, a user may press the snap-in clip 204 to subsequently release the cardiac sensing unit 106 from the frame 200. In implementations, as further shown in FIG. 3, the cardiac sensing unit 106 may include positioning tabs 206 that facilitate the attachment process between the cardiac sensing unit 106 and the adhesive patch 108. For example, the positioning tabs 206 may guide a user to insert the cardiac sensing unit 106 onto the correct portion of the frame 200 such that the cardiac sensing unit 106 can then be coupled, connected, or snapped into the frame 200 using the snap-in clip 204, as shown in FIG. 4.

Referring back to FIG. 1, an adhesive patch 108 housing the cardiac sensing unit 106 may be placed on predetermined locations on the body of the patient 100 as illustrated. As an example, the adhesive patch 108 may be placed on the side of the patient's thorax (e.g., under the patient's armpit, level with a circumferential line running across the patient's chest). As another example, the adhesive patch 108 may be placed on the front of the patient's thorax (e.g., to the left of a medial line running between the patient's collarbone, for patients without dextrocardia). However, the adhesive patch 108 may also be placed on any part of the patient's thorax that allows for efficient monitoring and recording of signals from and/or associated with the patient 100. For instance, the adhesive patch 108 may be placed over the patient's sternum, over a lower portion of the patient's front or side thorax (e.g., under the patient's sternum), and/or the like.

The cardiac sensing unit 106 and adhesive patch 108 are configured for long-term and/or extended use or wear by, or attachment or connection to, the patient 100. For example, devices as described herein are capable of being continuously used or continuously worn by, or attached or connected to, the patient 100 without substantial interruption (e.g., for 24 hours, 2 days, 5 days, 7 days, 2 weeks, 30 days or 1 month, or beyond such as multiple months or even years). In some implementations, such devices may be removed for a period of time before use, wear, attachment, or connection to the patient 100 is resumed. As an illustration, the cardiac sensing unit 106 may be removed for charging, to carry out technical service, to update the device software or firmware, for the patient 100 to take a shower, and/or for other reasons or activities without departing from the scope of the examples described herein. As another illustration, the patient 100 may remove a used adhesive patch 108, as well as the cardiac sensing unit 106, so that the patient 100 may adhere a new adhesive patch 108 to their body and attach the cardiac sensing unit 106 to a new adhesive patch 108. Such substantially or nearly continuous use, monitoring, or wear as described herein may nonetheless be considered continuous use, monitoring, or wear.

For example, in implementations, the adhesive patch 108 may be designed to maintain attachment to skin of the patient 100 for several days (e.g., in a range from about 4 days to about 10 days, from about 3 days to about 5 days, from about 5 days to about 7 days, from about 7 days to about 10 days, from about 10 days to about 14 days, from about 14 days to about 30 days, etc.). After the period of use, the adhesive patch 108 can be removed from the patient's skin and the cardiac sensing unit 106 can be removed from the adhesive patch 108. The cardiac sensing unit 106 can then be removably coupled, connected, or snapped onto a new adhesive patch 108 and reapplied to the patient's skin.

As further shown in FIG. 1, the wearable cardiac sensing device 102 may include a portable gateway 110 configured to facilitate communication between the wearable cardiac sensing device 102 and the remote server 104. In implementations, the portable gateway 110 is configured to receive data and/or signals provided by the wearable cardiac sensing device 102 and transmit the data and/or signals to the remote server 104. As an illustration, in implementations, the portable gateway 110 may communicate with the remote server 104 via Ethernet, via Wi-Fi, via near-field communication (NFC), via radiofrequency, via cellular networks, via Bluetooth®-to-TCP/IP access point communication, and/or the like. Accordingly, the portable gateway 110 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, the wearable cardiac sensing device 102 and/or the portable gateway 110 may include communications circuitry that is part of an Internet of Things (IoT) and communicate with each other and/or the remote server 104 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

Alternatively, in other implementations, the wearable cardiac sensing device 102 may be configured to transmit data and/or signals directly to the remote server 104 instead of, or in addition to, transmitting the signals to the portable gateway 110. Accordingly, the wearable cardiac sensing device 102 may be in wired or wireless communication with the remote server 104. As an illustration, the wearable cardiac sensing device 102 may communicate with the remote server 104 via Ethernet, via Wi-Fi, via near-field communication (NFC), via radiofrequency, via cellular networks, via Bluetooth®-to-TCP/IP access point communication, and/or the like. Further, in some implementations, the cardiac event risk assessment system may not include the portable gateway 110. In such implementations, the wearable cardiac sensing device 102 may perform the functions of the portable gateway 110 described herein. Additionally, in implementations where the wearable cardiac sensing device 102 is configured to communicate directly with the remote server 104, the wearable cardiac sensing device 102 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, as indicated above, the communications circuitry in the wearable cardiac sensing device 102 may be part of an IoT and communicate with the remote server 104 via IoT protocols for handling secure (e.g., encrypted) messaging and routing.

The wearable cardiac risk assessment system may also include a charger 112, as further shown in FIG. 1. In implementations, the charger 112 includes charging cradles configured to hold and recharge the cardiac sensing unit 106 and the portable gateway 110. Alternatively, in some implementations, the cardiac risk assessment system may not include the portable gateway 110, and accordingly, the charger 112 may be configured to hold the cardiac sensing unit 106 alone. In implementations, the cardiac sensing unit 106 and the portable gateway 110 may have separate chargers, or one or both of the cardiac sensing unit 106 and the portable gateway 110 may have removable batteries that may be replaced and/or recharged.

The remote server 104 is configured to receive and process the data and/or signals received from the wearable cardiac sensing device 102. In implementations, the remote server 104 may be in electronic communication with a number of wearable cardiac sensing devices 102 and be configured to receive and process the data and/or signals received from all of the wearable cardiac sensing devices 102 in communication with the remote server 104. The remote server 104 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the data and/or signals received from the wearable cardiac sensing device 102. For example, the at least one processor of the remote server 104 may be implemented as a digital signal processor (DSP), such as a 24-bit DSP processor; as a multicore-processor (e.g., having two or more processing cores); as an Advanced RISC Machine (ARM) processor, such as a 32-bit ARM processor; and/or the like. The at least one processor of the remote server 104 can execute an embedded operating system and further execute services provided by the operating system, where these services can be used for file system manipulation, display and audio generation, basic networking, firewalling, data encryption, communications, and/or the like. The database may be implemented as flash memory, solid state memory, magnetic memory, optical memory, cache memory, combinations thereof, and/or others. In various implementations, the remote server 104 may use the data received from the wearable cardiac sensing device 102 to determine a current status of cardiotoxicity in the patient 100, as described in further detail below. Alternatively, in some implementations, the wearable cardiac sensing device 102 may perform some or all of the analysis described herein as being performed by the remote server 104 (e.g., with the wearable cardiac sensing device 102 transmitting an indication of an abnormal cardiac event in the patient 100 or another output to the remote server 104, for instance, via the portable gateway 110).

The remote server 104 is configured to receive and process the data and/or signals received from the wearable cardiac sensing device 102. In implementations, the remote server 104 may be in electronic communication with a number of wearable cardiac sensing devices 102 and be configured to receive and process the data and/or signals received from all of the wearable cardiac sensing devices 102 in communication with the remote server 104. The remote server 104 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the data and/or signals received from the wearable cardiac sensing device 102. For example, the at least one processor of the remote server 104 may be implemented as a digital signal processor (DSP), such as a 24-bit DSP processor; as a multicore-processor (e.g., having two or more processing cores); as an Advanced RISC Machine (ARM) processor, such as a 32-bit ARM processor; and/or the like. The at least one processor of the remote server 104 can execute an embedded operating system and further execute services provided by the operating system, where these services can be used for file system manipulation, display and audio generation, basic networking, firewalling, data encryption, communications, and/or the like. The database may be implemented as flash memory, solid state memory, magnetic memory, optical memory, cache memory, combinations thereof, and/or others.

As shown in FIG. 1, in implementations, the cardiac event risk assessment system further includes one or more user interfaces, such as technician interfaces 114 and caregiver interfaces 116. The technician interfaces 114 and caregiver interfaces 116 are in electronic communication with the remote server 104 through a wired or wireless connection. For instance, the technician interfaces 114 and caregiver interfaces 116 may communicate with the remote server 104 via Wi Fi, via Ethernet, via cellular networks, and/or the like. Additionally, as shown, at least some of the technician interfaces 114 may be in electronic communication with at least some of the caregiver interfaces 116 through a wired or wireless connection, such as via Wi-Fi, via Ethernet, via cellular networks, and/or the like. The technician interfaces 114 and the caregiver interfaces 116 may include, for example, desktop computers, laptop computers, and/or portable personal digital assistants (e.g., smartphones, tablet computers, etc.).

In implementations, the technician interfaces 114 are configured to electronically communicate with the remote server 104 for the purpose of viewing and analyzing information gathered from one or more wearable cardiac sensing devices 102. For example, a technician interface 114 may provide one or more instructions to the remote server 104 to prepare a report on data and/or signals received from a given wearable cardiac sensing device 102 for a certain time period. Accordingly, a technician interface 114 may include a computing device having a processor communicably connected to a memory and a visual display. The technician interface 114 may display to a user of the technician interface 114 (e.g., a technician) data received from a wearable cardiac sensing device 102 and/or information computed from the data and/or signals received from wearable cardiac sensing device 102, described in further detail below. The user of the technician interface 114 may then provide one or more inputs to the remote server 104 to guide the remote server 104 in, for example, preparing a report for a patient 100.

As an illustration, a user of a technician interface 114 may select a time period to use for a report, and the remote server 104 may prepare a report corresponding to the selected time period. As another example, a user of a technician interface 114 may select types of data to be included in a report, such as risk assessments as described in more detail below. The remote server 104 may then prepare a report according to the types of data selected by the user. As another example, a user of a technician interface 114 may view a report prepared by the remote server 104 and draft a summary of the report to be included in a summary section for the report. Alternatively, in implementations, the remote server 104 may analyze, summarize, etc. the data and/or signals received from a wearable cardiac sensing device 102 with minimal or no input or interaction with a technician interface 114. In this way, the remote server 104 may analyze, summarize, etc. the information gathered from a wearable cardiac sensing device 102 and prepare a report on this information through a completely or mostly automated process.

The caregiver interfaces 116 are configured to electronically communicate with the remote server 104 for the purpose of viewing information on various patients 100 using a wearable cardiac sensing device 102. As such, a caregiver interface 116 may include a computing device having a processor communicably connected to a memory and a visual display. The caregiver interface 116 may display to a user of the caregiver interface 116 (e.g., a physician, a nurse, or other caregiver), for example, risk assessments for the patient 100 using the wearable cardiac sensing device 102, as described in further detail below. In implementations, the caregiver interface 116 may display to a user one or more reports summarizing risk of having a future cardiac events in the patient 100, such as one or more reports prepared by the remote server 104 (e.g., based on inputs from one or more technician interfaces 114). In implementations, the user of a caregiver interface 116 may be able to interact with the information displayed on the caregiver interface 116. As an example, the user of a caregiver interface 116 may be able to select a portion of a patient report and, in response, be able to view additional information relating to the selected portion of the report. Additional information may include, for instance, ECG data or cardiovibration data from the wearable cardiac sensing device 102 used to prepare the report and/or the like. In implementations, the user of the caregiver interface 116 may instead view a static patient report that does not have interactive features.

In implementations, a technician interface 114 and/or a caregiver interface 116 may be a specialized interface configured to communicate with the remote server 104. As an example, the technician interface 114 may be a specialized computing device configured to receive preliminary patient reports from the remote server 104, receive inputs from a user to adjust the preliminary report, and transmit the inputs back to the remote server 104. The remote server 104 then uses the inputs from the technician interface 114 to prepare a finalized patient report, which the remote server 104 also transits to the technician interface 114 for review by the user. As another example, the caregiver interface 116 may be a specialized computing device configured to communicate with the remote server 104 to receive and display patient reports, as well as other information regarding patients 100 using a wearable cardiac sensing device 102.

In implementations, a technician interface 114 and/or a caregiver interface 116 may be a generalized user interface that has been adapted to communicate with the remote server 104. To illustrate, the technician interface 114 may be a computing device (e.g., a laptop, a portable personal digital assistant such as a smartphone or tablet, etc.) executing a technician application that configures the computing device to communicate with the remote server 104. For example, the technician application may be downloaded from an application store or otherwise installed on the computing device. Accordingly, when the computing device executes the technician application, the computing device is configured to establish an electronic communication link with the remote server 104 to receive and transmit information regarding patients 100 using a wearable cardiac sensing device 102. Similarly, the caregiver interface 116 may be a computing device (e.g., a laptop, a portable personal digital assistant such as a smartphone or tablet, etc.) executing a caregiver application that configures the computing device to communicate with the remote server 104. The caregiver application may be similarly downloaded from an application store or otherwise installed on the computing device and, when executed, may configure the computing device to establish a communication link with the remote server 104 to receive and display information on patients 100 using a wearable cardiac sensing device 102.

The application store is typically included within an operating system of a computing device implementing a user interface. For example, in a device implementing an operating system provided by Apple Inc. (Cupertino, California), the application store can be the App Store, a digital distribution platform, developed and maintained by Apple Inc., for mobile apps on its iOS and iPadOS® operating systems. The application store allows a user to browse and download an application, such as the technician or caregiver application, developed in accordance with the Apple® iOS Software Development Kit. For instance, such technician or caregiver application may be downloaded on an iPhone® smartphone, an iPod Touch® handheld computer, or an iPad® tablet computer, or transferred to an Apple Watch® smartwatch. Other application stores may alternatively be used for other types of computing devices, such as computing devices operating on the Android® operating system.

In some implementations, the technician application and the caregiver application may be the same application, and the application may provide different functionalities to the computing device executing the application based on, for example, credentials provided by the user. For instance, the application may provide technician functionalities to a first computing device in response to authenticating technician credentials entered on the first computing device, and may provide caregiver functionalities to a second computing device in response to authenticating caregiver credentials entered on the second computing device. In other cases, the technician application and the caregiver application may be separate applications, each providing separate functionalities to a computing device executing them.

In implementations, the cardiac event risk assessment system shown in FIG. 1 may include other types of interfaces. To illustrates, in examples, the cardiac event risk assessment system may include patient interfaces. The remote server 104 and/or a technician interface 114 may provide a report on a patient 100 using a wearable cardiac sensing device 102 to this patient 100 via a patient interface. This patient report may be the same as a report provided to a caregiver via a caregiver interface 116, or this report may be different from the report provided to the caregiver via the caregiver interface 116. For instance, the report provided to a patient 100 may be an abridged version of a report prepared for the patient's caregiver, such as a report that summarizes the key information from the report prepared for the patient's caregiver. In various implementations, the patient interface may be configured similarly to and function similarly to the caregiver interface 116 discussed above. As an example, the patient interface may be similar to the caregiver interface 116 but with some restrictions on what is included in a patient report compared to a report prepared for the patient's caregiver, and/or with some restrictions on the functionalities of, for instance, an application executed by the patient interface that the patient 100 can use to access and view a patient report or other information on the patient 100.

Returning back to the wearable cardiac sensing device 102, as noted above the wearable cardiac sensing device 102 includes a number of physiological sensors configured to sense signals from and/or associated with the patient 100. To illustrate, in implementations of the wearable cardiac sensing device 102 that includes a cardiac sensing unit 106 and an adhesive patch 108, as described above with reference to FIG. 1, the physiological sensors may be implemented in the cardiac sensing unit 106 and/or implemented in the adhesive patch 108. For example, at least a portion of the physiological sensors may be integrated in the adhesive patch 108. As another example, at least a portion of the physiological sensors may be configured to be removably mounted onto the adhesive patch 108 (e.g., through being incorporated in or otherwise implemented via the cardiac sensing unit 106, which is configured to be removably mounted on to the adhesive patch 108 as discussed above).

In implementations, a number of ECG electrodes 202 may be embedded into the adhesive patch 108, as shown in FIG. 2. The ECG electrodes 202 are configured to sense one or more electrical signals indicative of ECG activity from a skin surface of the patient 100 (e.g., skin contacted directly or through a covering). The ECG electrodes 202 may be coupled to the cardiac sensing unit 106 by dedicated wiring within the adhesive patch 108, where the cardiac sensing unit 106 receives electrical signals sensed by the ECG electrodes 202 via the dedicated metal contacts or traces on the cardiac sensing unit 106 when the cardiac sensing unit 106 is mounted on the adhesive patch 108. Example ECG electrodes 202 include a metal electrode with an oxide coating, such as tantalum pentoxide electrodes. For instance, digital sensing electrodes can include skin-contacting electrode surfaces that may be deemed polarizable or non-polarizable depending on a variety of factors, including the metals and/or coatings used in constructing the electrode surface. All such electrodes can be used with the principles, techniques, devices, and systems described herein. As an illustration, the electrode surfaces can be based on stainless steel, noble metals such as platinum, or Ag—AgCl.

In examples, the ECG electrodes 202 can be used with an electrolytic gel dispersed between the electrode surface and the patient's skin. In other examples, the ECG electrodes 202 can be dry electrodes that do not need an electrolytic material or optionally can be used with an electrolytic material. For instances, such a dry electrode can be based on tantalum metal and have a tantalum pentoxide coating, as is described above. Such dry electrodes may be more comfortable for long-term monitoring applications, in various implementations.

In implementations, the ECG electrodes 202 can include additional components such as accelerometers, biovibration signal detecting devices, cardiovibrational sensors, or other measuring devices for additional parameters. For example, the ECG electrodes 202 may be configured to detect other types of physiological signals, such as thoracic fluid levels, heart vibrations, lung vibrations, respiration vibrations, patient movement, etc. Alternatively, or additionally, the cardiac sensing unit 106 and/or the adhesive patch 108 may include sensors or detectors separate from the ECG electrodes 202, such as separate motion detectors, biovibration sensors, cardiovibrational sensors, respiration sensors, temperature sensors, pressure sensors, and/or the like.

In some implementations, cardiovibrational sensors 203 may be included within the adhesive patch and separate from the ECG electrodes. Optionally, the cardiovibrational sensors 203 may be used in an implementation of a cardiac sensing unit that does not include ECG electrodes. Alternatively, the cardiovibrational sensors 203 may be incorporated into the ECG electrodes as discussed above. Cardiovibrational sensors 203 may be positioned within the adhesive patch so as to be in the optimal location to record physiological markers. For example, in some implementations the cardiovibrational sensors 203 may be positioned to be proximate the left sternal border of a patient. Cardiovibration sensors 203 may be configured to detect physiological signals such as cardiovibrations. The detected physiological signals may undergo processing locally, at the sensor, or remotely, at a server communicatively coupled to the cardiovibrational sensors 203. Examples of physiological signals that can be detected by the cardiovibration sensors 203 include signals corresponding to S1, S2, S3, and/or S4 heart sounds. In examples, cardiovibration sensors 203 may include accelerometers or piezo electric sensors.

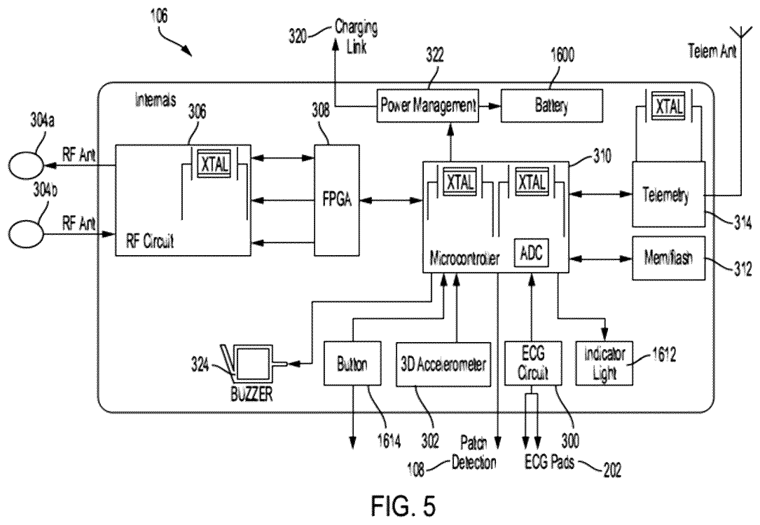

FIG. 5 illustrates an example electronic architecture for the cardiac sensing unit 106. The ECG circuit 300 may receive signals from the ECG electrodes 202 when the cardiac sensing unit 106 is attached to the adhesive patch 108, where the signals received from the ECG electrodes 202 include ECG waveforms sensed from the patient 100. In embodiments, the ECG electrodes 202 and cardiac sensing unit 106 may have a sampling rate in a range from about 250 Hz to about 500 Hz, from about 300 Hz to about 450 Hz, from about 350 Hz to about 400 Hz, including values and subranges therebetween. In embodiments, the cardiac sensing unit 106 may sample the signals from the ECG electrodes 202 after applying band-pass filtering by a 12 bit ADC. In implementations, during normal operation, the cardiac sensing unit 106 may transfer data from the ECG electrodes 202 to the remote server 104 “as-is,” where the remote server 104 uses the data for analysis as described in further detail below. Alternatively, in other implementations, the cardiac sensing unit 106 may perform at least some processing of the data from the ECG electrodes 202 (e.g., filtering of the data, analysis of the data, etc.). For example, the cardiac sensing unit 106 may generated digitized ECG signals from the data received from the ECG electrodes 202 and transmit the digitized ECG signals to the remote server 104 (e.g., directly or via the portable gateway 110). Similarly, in some implementations, the cardiac sensing unit 106 may perform at least some of the processing of the data form the cardiovibration sensors 203. For example, the cardiac sensing unit 106 may generate digitized cardiovibration signals from the data received from the cardiovibration sensors 203 and transmit the digitized cardiovibration signals to the remote server 104 (e.g., directly or via the portable gateway 110).

In implementations, the wearable cardiac sensing device 102 can be designed to include a digital front-end where analog signals sensed by skin-contacting electrode surfaces of a set of digital sensing electrodes are converted to digital signals for processing. In this respect, the ECG circuit 300 may be an ECG digitizing circuit configured to provide digitized ECG signals of the patient 100 based on the electrical signals sensed from the patient 100. Typical wearable, ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device (e.g., the microcontroller 310 discussed below) for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

Internally, the wearable cardiac sensing device 102 includes a processor operationally coupled to, for example, the ECG circuit 300. In embodiments, the processor may be implemented as a microcontroller 310, as shown in FIG. 5. The microcontroller 310 stores instructions specifying how measurements (e.g., ECG, accelerometer, RF, etc.) are taken, how obtained data are transmitted, how to relay a status of the cardiac sensing unit 106, how/when the cardiac sensing unit 106 can enter a sleep level, and/or the like. In implementations, the instructions may specify the conditions for performing certain types of measurements. For example, the instructions may specify that a sensor of and/or connected to the cardiac sensing unit 106 may not commence measurements (e.g., ECG measurements, RF measurements, respiration rate measurements, and/or the like) unless the patient 100 is at rest or maintaining a certain posture. As another example, the instructions may identify the conditions that have to be fulfilled before any measurements can commence, such as a sufficient attachment level between the cardiac sensing unit 106 and the adhesive patch 108, or a sufficient attachment level between the adhesive patch 108 and the surface of the patient's body to which the adhesive patch 108 is attached. In implementations, the microcontroller 310 may have internal and/or external non-volatile memory banks (e.g., memory 312) that can be used for measurement directories and data, scheduler information, and/or a log of actions and errors. This non-volatile memory may allow for retaining data and status information in the case of a total power down. In implementations, instead of a microcontroller 310 as described above, the cardiac sensing unit 106 may include a microprocessor connected to a separate non-volatile memory, such as the memory 312.

The memory 312 may also be configured as a non-transitory memory configured to store data and/or signals of the cardiac sensing unit 106. For instance, the memory 312 may be configured to store digitized ECG signals of the patient 100.

The cardiac sensing unit 106 may further be able to establish wireless communications channels with other devices, such as the portable gateway 110 and/or the remote server 104, using a telemetry or wireless communications circuit 314. For example, the wireless communications circuit 314 may be a Bluetooth® unit. Additionally, or alternatively, the wireless communications circuit 314 may include other modules facilitating other types of wireless communication (e.g., Wi-Fi, cellular, etc.). The cardiac sensing unit 106 may transmit data to the remote server 104 using the wireless communication circuit 314. In implementations, the cardiac sensing unit 106 may transmit data indirectly to the remote server 104, such as by transmitting the signals and/or data to the portable gateway 110, with the portable gateway 110 transmitting the data received from the cardiac sensing unit 106 to the remote server 104. In implementations, the cardiac sensing unit 106 may instead transmit the data directly to the remote server 104.

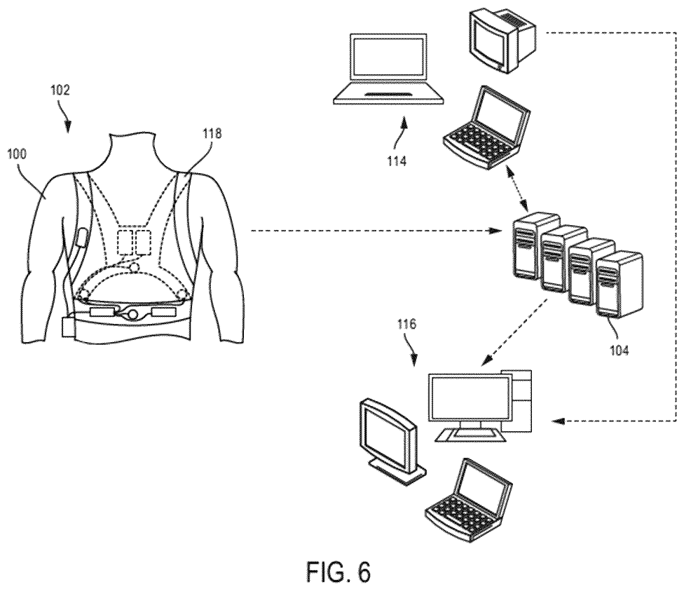

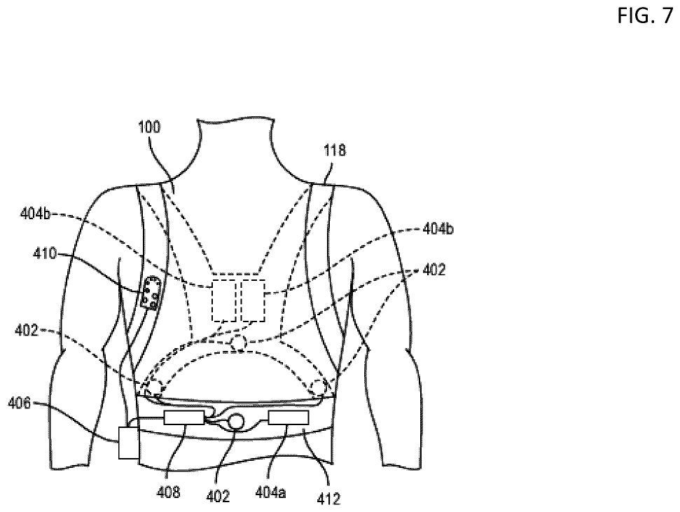

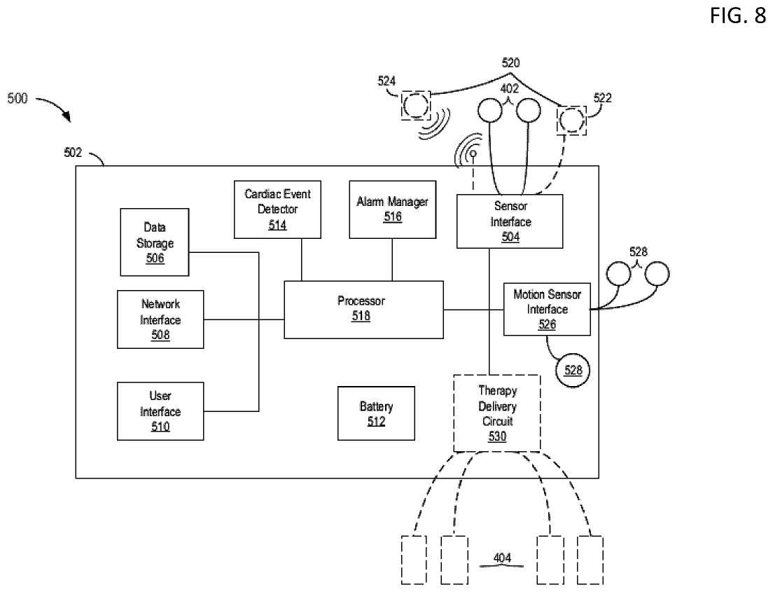

In implementations, the wearable cardiac sensing device 102 may be configured as a different wearable device from the example embodiments illustrated in FIGS. 1-5. For instance, FIG. 6 illustrates another example of a cardiac event risk assessment system for improved ECG morphological analysis for determining abnormal cardiac events in a patient 100, according to implementations disclosed herein. The cardiac event risk assessment system of FIG. 6 includes another embodiment of the wearable cardiac sensing device 102 configured to be bodily-attached to the patient 100. The wearable cardiac sensing device 102 according to FIG. 6 is a garment-based sensing device 118 configured to be worn about the patient's torso, where various electronic components are temporarily and/or permanently mounted to the garment of the garment-based sensing device 118 (as described in further detail with respect to FIG. 7 below). Such a garment-based sensing device 118 can be, for example, capable of and designed for moving with the patient 100 as the patient 100 goes about their daily routine. For example, the garment-based sensing device 118 as described herein with respect to FIG. 6 (as well as FIGS. 7 and 8 below) can be a wearable cardioverter defibrillator, or wearable defibrillator, configured to be bodily-attached to the patient 100. In one example scenario, such wearable defibrillators can be worn nearly continuously or substantially continuously for a week, two weeks, a month, or two or three months at a time. During the period of time in which it is worn by the patient 100, the wearable defibrillator can be configured to continuously or substantially continuously monitor the vital signs of the patient 100 and, upon determining that the patient 100 is experiencing a treatable arrhythmia, deliver one or more therapeutic electrical pulses to the patient 100. For example, such therapeutic shocks can be pacing, defibrillation, cardioversion, or transcutaneous electrical nerve stimulation (TENS) pulses.

The cardiac event risk assessment system shown in FIG. 6 (including the remote server 104, technician interface(s) 114, and caregiver interface(s) 116) may generally function similarly to the cardiac event risk assessment system shown and described above with respect to FIG. 1. Similar to the example wearable cardiac sensing device 102 shown in FIG. 2, the example wearable cardiac sensing device 102 shown in FIG. 6 is configured for long-term and/or extended use or wear by, or attachment or connection to, the patient 100, as discussed above. Such substantially or nearly continuous use, monitoring, or wear similar to the usages described above may nonetheless be considered continuous use, monitoring, or wear.

FIG. 7 illustrates the garment-based sensing device 118 in more detail, according to various implementations. As shown in FIG. 7, the garment-based sensing device 118 can include one or more of the following: a garment 400 configured to be worn around the patient's torso, a plurality of physiological sensors, one or more therapy electrodes 404a and 404b (collectively referred to herein as therapy electrodes 404), a medical device controller 406, a connection pod 408, a patient interface pod 410, a belt 412, or any combination of these components. In examples, at least some of the components of the garment-based sensing device 118 (e.g., at least a portion of the physiological sensors, including the ECG electrodes 402; at least a portion of the therapy electrodes 404; etc.) are configured to be mounted on or affixed to the garment 400, such as by mating hooks, hook-and-loop fabric strips, receptacles (e.g., pockets), and the like. For example, the ECG electrodes 402 may be mounted on the garment 400 by hook-and-loop fabric strips on the electrodes 402 and the garment 400, and the therapy electrodes 404 may be mounted on the garment 400 by being inserted into receptacles of the garment 400 (e.g., into pockets of the garment 400). In examples, at least some of the components of the garment-based sensing device 118 (e.g., at least a portion of the physiological sensors, including the ECG electrodes 402; at least a portion of the therapy electrodes 404; etc.) can be permanently integrated into the garment 400, such as being sewn into or permanently adhered onto the garment 400. In examples, at least some of the components may be connected to each other through external cables, through sewn-in connections (e.g., wires woven into the fabric of the garment 400 or between layers of the garment 400), through conductive fabric of the garment 400, and/or the like.

The medical device controller 406 can be operatively coupled to the ECG electrodes 402, which can be affixed to the garment 400 (e.g., assembled into the garment 400 or removably attached to the garment 400, for example, using hook-and-loop fasteners) or permanently integrated into the garment 400. In implementations, the medical device controller 406 is also operatively coupled to the therapy electrodes 404. The therapy electrodes 404 may be similarly assembled into the garment 400 (e.g., into pockets or other receptacles of the garment 400) or permanently integrated into the garment 400. As shown in FIG. 4, the ECG electrodes 402 and/or the therapy electrodes 404 can be directly operatively coupled to the medical device controller 406 through the connection pod 408. Component configurations other than those shown in FIG. 7 are also possible. For example, the ECG electrodes 402 can be configured to be attached at various positions about the body of the patient 100. In implementations, at least one of the ECG electrodes 402 and/or at least one of the therapy electrodes 404 can be included on a single integrated patch and adhesively applied to the patient's body. In implementations, at least one of the ECG electrodes 402 and/or at least one of the therapy electrodes 404 can be included in multiple patches and adhesively applied to the patient's body. Such patches may be in wired (e.g., directly or via the connection pod 408) or wireless connection with the medical device controller 406. Similar implementations may also be extended to the non-ECG physiological sensors of the garment-based sensing device 118.

The ECG electrodes 402 are configured to detect one or more cardiac signals, such as electrical signals indicative of ECG activity from a skin surface of the patient 100. In implementations, the ECG electrodes 402 of the garment-based sensing device 118 may be configured similarly to the ECG electrodes 202 of the adhesive patch 108 described above. In implementations, the therapy electrodes 404 can also be configured to include sensors that detect ECG signals as well as, or in the alternative from, other physiological signals from the patient 100. The connection pod 408 can, in various examples, include a signal processor configured to amplify, filter, and digitize these cardiac signals prior to transmitting the cardiac signals to the medical device controller 406.

Additionally, the therapy electrodes 404 can be configured to deliver one or more therapeutic cardioversion/defibrillation shocks to the body of the patient 100 when the medical device controller 406 determines that such treatment is warranted based on the signals detected by the ECG electrodes 402 and processed by the medical device controller 406. Example therapy electrodes 404 can include conductive metal electrodes such as stainless-steel electrodes. In implementations, the therapy electrodes 404 may also include one or more conductive gel deployment devices configured to deliver conductive gel between the metal electrode and the patient's skin prior to delivery of a therapeutic shock.

In implementations, the medical device controller 406 may also be configured to warn the patient 100 prior to the delivery of a therapeutic shock, such as via output devices integrated into or connected to the medical device controller 406, the connection pod 408, and/or the patient interface pod 410. The warning may be auditory (e.g., a siren alarm, a voice instruction indicating that the patient 100 is going to be shocked), visual (e.g., flashing lights on the medical device controller 406), haptic (e.g., a tactile, buzzing alarm generated by the connection pod 408), and/or the like. If the patient 100 is still conscious, the patient 100 may be able to delay or stop the delivery of the therapeutic shock. For example, the patient 100 may press one or more buttons on the patient interface pod 410 and/or the medical device controller 406 to indicate that the patient 100 is still conscious. In response to the patient 100 pushing the one or more buttons, the medical device controller 406 may delay or stop the delivery of the therapeutic shock.

In implementations, a garment-based sensing device 118 as described herein can be configured to switch between a therapeutic mode and a monitoring mode such that, when in the monitoring mode, the garment-based sensing device 118 is configured to only monitor the patient 100 (e.g., not provide or perform any therapeutic functions). For example, in such implementations, therapeutic components such as the therapy electrodes 404 and associated circuitry may be decoupled from (or coupled to) or switch out of (or switched into) the garment-based sensing device 118. As an illustration, a garment-based sensing device 118 can have optional therapeutic elements (e.g., defibrillation and/or pacing electrode components and associated circuitry) that are configured to operate in a therapeutic mode. The optional therapeutic elements may be physically decoupled from the garment-based sensing device 118 as a means to convert the garment-based sensing device 118 from a therapeutic mode into a monitoring mode. Alternatively, the optional therapeutic elements may be deactivated (e.g., by means of a physical or software switch), essentially rendering the garment-based sensing device 118 as a monitoring-only device for a specific physiological purpose for the particular patient 100. As an example of a software switch, an authorized person may be able to access a protected user interface of the garment-based sensing device 118 and select a preconfigured option or perform some other user action via the user interface to deactivate the therapeutic elements of the garment-based sensing device 118.

FIG. 8 illustrates a sample component-level view of a medical device controller 500 included in a garment-based sensing device 118. The medical device controller 500 is an example of the medical device controller 406 shown in FIG. 7 and described above. As shown in FIG. 8, the medical device controller 500 may include a housing 502 configured to house a number of electronic components, including a sensor interface 504, a data storage 506, a network interface 508, a user interface 510, at least one battery 512 (e.g., positioned within a battery chamber configured for such a purpose), a cardiac event detector 514, an alarm manager 516, at least one processor 518, and a therapy delivery circuit 530. As described above, in some implementations, the garment-based sensing device 118 may include like components to those described above but may not include therapeutic components. That is, in some implementations, the garment-based sensing device 118 can include ECG monitoring components and not provide therapy to the patient 100. Accordingly, the garment-based sensing device 118 may not include the therapy delivery circuit 530 and the therapy electrodes 404 (shown in dotted lines), or the therapy delivery circuit 530 and the therapy electrodes 404 may be disconnectable from the rest of the garment-based sensing device 118.

In implementations, the processor 518 includes one or more processors (or one or more processor cores) that are each configured to perform a series of instructions that result in the manipulation of data and/or the control of the operation of the other components of the medical device controller 500. In implementations, when executing a specific process (e.g., monitoring sensed electrical data of the patient 100), the processor 518 can be configured to make specific logic-based determinations based on input data received. The processor 518 may be further configured to provide one or more outputs that can be used to control or otherwise inform subsequent processing to be carried out by the processor 518 and/or other processors or circuitry to which the processor 518 is communicably coupled. Thus, the processor 518 reacts to a specific input stimulus in a specific way and generates a corresponding output based on that input stimulus. In example cases, the processor 518 can proceed through a sequence of logical transitions in which various internal register states and/or other bit cell states internal or external to the processor 518 may be set to logic high or logic low.

As referred to herein, the processor 518 can be configured to execute a function where software is stored in a data store (e.g., the data storage 506) coupled to the processor 518, the software being configured to cause the processor 518 to proceed through a sequence of various logic decisions that result in the function being executed. The various components that are described herein as being executable by the processor 518 can be implemented in various forms of specialized hardware, software, or a combination thereof. For example, the processor 518 can be a digital signal processor (DSP) such as a 24-bit DSP processor. As another example, the processor 518 can be a multi-core processor, e.g., having two or more processing cores. As another example, the processor 518 can be an Advanced RISC Machine (ARM) processor, such as a 32-bit ARM processor. The processor 518 can execute an embedded operating system and further execute services provided by the operating system, where these services can be used for file system manipulation, display and audio generation, basic networking, firewalling, data encryption, communications, and/or the like.

The data storage 506 can include one or more of non-transitory media, such as flash memory, solid state memory, magnetic memory, optical memory, cache memory, combinations thereof, and others. The data storage 506 can be configured to store executable instructions and data used for operation of the medical device controller 500. In implementations, the data storage 506 can include sequences of executable instructions that, when executed, are configured to cause the processor 518 to perform one or more functions. Additionally, the data storage 506 can be configured to store information such as digitized ECG signals of the patient 100.

In examples, the network interface 508 can facilitate the communication of information between the medical device controller 502 and one or more devices or entities over a communications network. For example, the network interface 508 can be configured to communicate with the remote server 104 or other similar computing device. Using the network interface 508, the garment-based sensing device 118 may transmit, for example, ECG signals, other physiological signals, indications of abnormal cardiac events, etc., to the remote server 104. In implementations, the network interface 508 can include communications circuitry for transmitting data in accordance with a Bluetooth® wireless standard for exchanging such data over short distances to an intermediary device(s) (e.g., a base station, “hotspot” device, smartphone, tablet, portable computing device, and/or other device in proximity with the garment-based sensing device 118, such as a device similar to the portable gateway 110). The intermediary device(s) may in turn communicate the data to the remote server 104 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 104 over a Wi-Fi communications link based on the IEEE 802.11 standard. In implementations, the network interface 508 may be configured to instead communicate directly with the remote server 104 without the use of intermediary device(s). In such implementations, the network interface 508 may use any of the communications links and/or protocols provided above to communicate directly with the remote server 104.

The sensor interface 504 can include physiological signal circuitry that is coupled to one or more externally applied sensors 520. The externally applied sensors 520 may include, for example, one or more externally applied physiological sensors. As shown, the sensors may be coupled to the medical device controller 500 via a wired or wireless connection. The externally applied sensors 520 may include the ECG electrodes 402 configured to sense one or more electrical signals indicative of ECG activity from the skin surface of the patient 100, as well as one or more non-ECG sensors such as a cardiovibration sensor 522 and a tissue fluid monitor 524 (e.g., configured similarly to the thoracic fluid sensor implemented through the at least one RF antenna 304a, 304b and RF circuitry 306 discussed in further detail below with reference to FIG. 5). In implementations, the sensor interface 504 and/or the processor 518 may be configured to provide digitized ECG signals of the patient 100 based on electrical signals sensed by the ECG electrodes 402. In this sense, the sensor interface 504 and/or the processor 518 may be considered an ECG digitizing circuit. The digitized ECG signals of the patient 100 may be stored in the data storage 506.

Regardless of the embodiment of the wearable cardiac sensing device 102 used, a processor in communication with the wearable cardiac sensing device 102 may be configured to process ECG data and/or cardiovibration data gathered by the wearable cardiac sensing device 102 to determine risk of having future cardiac events in the patient 100.

For example, cardiac ischemia is characterized as a lack of blood flow and oxygen to the heart muscle, which may cause damage to the tissues in the heart muscle. In particular, a lack of oxygen to regions of the heart can cause scarring in the heart tissue which can change the stiffness of heart muscle tissues. As the stiffness of the heart muscles is altered, vibrations experienced by the heart muscles may be altered. Changes in the vibrations experienced by the heart muscles can be observed in the frequencies associated with the resulting sounds produced by the heart muscles. Accordingly, implementations of the cardiac event risk assessment system described herein may analyze frequencies associated with sounds produced by the heart muscles to determine whether a patient has a higher risk for cardiac ischemia. Cardiac ischemia is often also a precursor to sudden cardiac arrest, which is the sudden loss of all heart activity. Accordingly, an analysis of the frequency associated with cardiovibrations can also be used to determine whether the patient has a higher risk for sudden cardiac arrest.

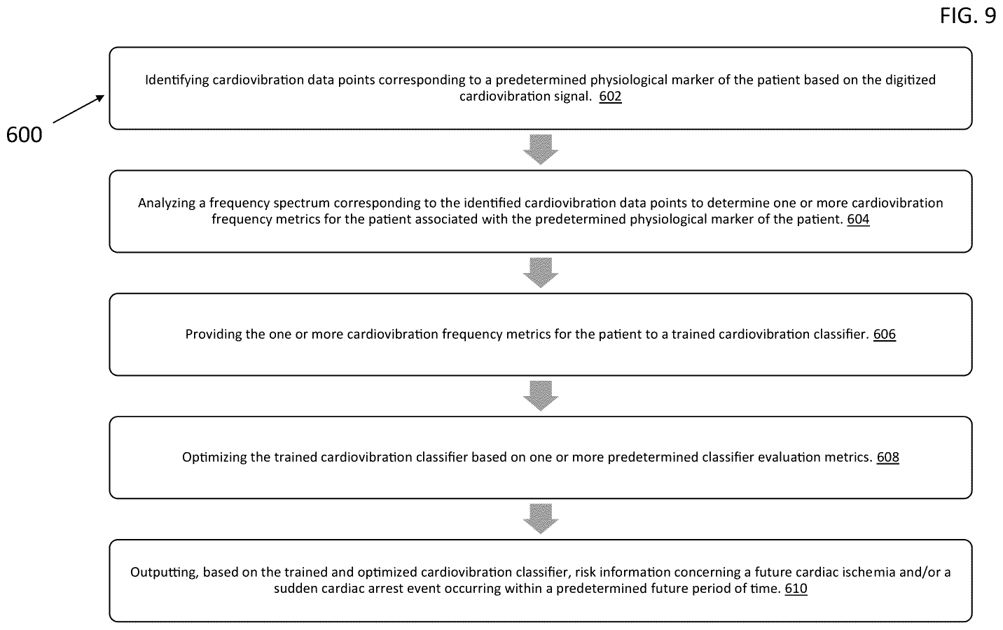

FIG. 9 illustrates a sample process flow in an cardiac event risk assessment system that analyzes cardiovibration data to generate and output risk information concerning a future cardiac ischemic or sudden cardiac arrest event occurring in a patient. The sample process 600 shown in FIG. 9 can be implemented by a processor in communication with the wearable cardiac sensing device 102. In implementations, the processor may be part of the wearable cardiac sensing device 102. For example, where the wearable cardiac sensing device 102 includes a cardiac sensing unit 106 and a portable gateway 110, the processor may be implemented by the microcontroller 310 of the cardiac sensing unit 106. As another example, the processor may be implemented in the portable gateway 110. As another example, where the wearable cardiac sensing device 102 includes a garment-based sensing device 118, the processor may be implemented by the processor 518. As another example, the processor may be implemented in the remote server 104. As another example, the processor may be implemented partially by a processor at the wearable cardiac sensing device 102 and partially by a processor at the remote server 104.

The processor is configured to identify cardiovibration data points corresponding to a predetermined physiological marker of the patient based on the digitized cardiovibration signal at step 602.

A digitized cardiovibration signal is obtained by the processor from digitized cardiovibration signals stored in a non-transitory memory of the wearable cardiac sensing device 102. As such, the processor receives the digitized cardiovibration signals from the wearable cardiac sensing device 102 (e.g., via a transmission of the wearable cardiac sensing device 102) or retrieves the digitized cardiovibration signals from the non-transitory memory of the wearable cardiac sensing device 102. For example, the processor may receive or retrieve a predetermined timeseries of cardiovibration data corresponding to the data points that form a segment of a digitized ECG signal. Alternatively, or additionally, the processor may receive or retrieve a predetermined timeseries of cardiovibration data corresponding to a heartbeat. The digitized cardiovibration signals may be pre-segmented (e.g., by the wearable cardiac sensing device 102, such as at the cardiac sensing unit 106, at the portable gateway 110, or at the controller 500 of the garment-based sensing device 118). Alternatively, the digitized cardiovibration signal segments may be segmented by the processor, for instance, at the time the processor receives or retrieves the digitized cardiovibration signals. The segments may be generated according to a predetermined time period (e.g., 60 seconds of cardiovibration data, 90 seconds of cardiovibration data, 120 seconds of cardiovibration data, 5 minutes of cardiovibration data, and/or the like), according to a transmission schedule (e.g., segmented to facilitate transmission of the digitized cardiovibration signals to the remote server 104), according to a symptom event recorded by the wearable cardiac sensing device 102, such that each segment represents a single ECG cycle, heartbeat, and/or the like.

The processor is configured to identify cardiovibration data points corresponding to a predetermined physiological marker of the patient. Examples of physiological markers of the patient include S1, S2, S3, and/or S4 heart beat sounds. S1 sounds may correspond to the vibrational sound made by the heart during closure of the atrioventricular (AV) valves (e.g., the mitral and tricuspid valves). S2 sounds may correspond to the vibrational sound made by the heart resulting from the closure of the semilunar valves (e.g., the pulmonary and aortic valves) which may also correspond to the beginning of diastole. S3 may correspond to vibrational sounds made by the heart due to a rapid filling of ventricles with blood from the atria while the ventricle wall is not relaxed. S4 may correspond to vibrational sounds made by the heart due to the rapid filling of ventricles with blood from the atria due to atrial contraction.

In some implementations, the processor is configured to identify cardiovibration data points corresponding to a S1 region of a heartbeat. For example, the S1 region may be determined based on its relative position with the R-peak of a QRS signal. The processor is configured to obtain the digitized cardiovibration signal, identify heart beats within the obtained digitized cardiovibration signal by identifying local R-peaks in an ECG signal and looking for corresponding cardiovibration features in the cardiovibration signal occurring at the same time, and identify a set of points adjacent to the identified local R-peak which correspond to a S1 region for each identified heartbeat.

Similarly, in some implementations the processor is configured to identify cardiovibration data points corresponding to a S2 region of a heartbeat. For example, the S2 region may be determined based on its relative position with the T-wave. The processor is configured to obtain the digitized cardiovibration signal, identify heart beats within the obtained digitized cardiovibration signal by identifying local R-peaks in an ECG signal and looking for corresponding cardiovibration features in the cardiovibration signal occurring at the same time within the obtained digitized cardiovibration signal, identify a corresponding T-wave for each identified local R-peak, and identify a set of points adjacent to the identified T-wave corresponding to a S2 region for each identified heartbeat.

In some implementations, the processor is configured to identify cardiovibration data points corresponding to both a S1 region and a S2 region of a heartbeat. For example, the processor is configured to isolate both S1 and S2 heart sounds for each heartbeat. For example the cardiovibration data points are extracted from the cardiovibration signal. An ECG signal obtained for the same time period as the cardiovibration signal may be used to identify the local R-peaks and T-waves. Times corresponding to the local R-peaks and T-waves in the ECG signal may be used to determine the timing for datapoint extraction for the cardiovibration signal.

In some implementations, a Zeelenberg R peak finding algorithm or other R-peak finding algorithm may be applied to find R-peaks. For example, ECG signal data may be passed through a differentiator with a 50 Hz notch filter. Then the ECG signal data may be passed through a digital band-pass filter with a 6-25 Hz passband and 35-100 Hz stopband. Opposite polarity thresholds may be applied to the band-passed signal data. R-peaks may be determined by count the number of threshold crossings in a 160 millisecond region. For example, if no threshold crossings are present, the ECG signal data corresponds to a shift, if one threshold crossing is present with cuts then the ECG signal data corresponds to a QRS wave. If more than one threshold crossing is present then the ECG signal data is noisy. By applying a Zeelenberg or other R-peak finding algorithm, R-peaks can be located within the ECG signal and heart beats can be separated using the located R-peaks. In some implementations, heart beats with timing greater than 250 milliseconds can be discarded.

Timing corresponding to the S1 region and/or S2 region can be determined for each heartbeat determined from the ECG signal using the algorithms described above. For example, an S1 region may span the first 0 to 20% of the heartbeat duration. Similarly, the S2 region may be based on the location of the T-peak and span approximately between 50% to 70% of the beat duration. S2 regions may vary on a patient by patient basis and the region corresponding to the S2 may be determined accordingly. Once timing corresponding to the S1 and/or S2 region is determined based on the ECG heartbeat data the cardiovibration data points corresponding to the determined timings can be used to identify cardiovibration datapoints corresponding to S1, S2, or other predetermined physiological markers.

In some implementations, heart beats are separated using the located R peaks and beats longer than 250 ms are discarded. The frequency power spectrum for each beat is extracted using an NFFT value of 100. The S1 region is defined as the first time bin in the spectrum, typically between 0% and 20% of the beat duration. The S2 region is defined based upon the location of the T peak, typically between 50% and 70% of the beat duration. Because the location of the S2 region can vary from patient to patient, in some implementations the S2 region may be calibrated on a per patient basis.

Turning back to FIG. 9, at step 604, the processor is configured to analyze a frequency spectrum corresponding to the identified cardiovibration data points to determine one or more cardiovibration frequency metrics for the patient associated with the predetermined physiological marker of the patient. For example, each isolated set of cardiovibration data points corresponding to a predetermined physiological marker (e.g., S1, S2) can be analyzed by applying a Fast Fourier Transform (FFT) and/or a FFT Power Spectrum to the identified cardiovibration data points. The processor is configured to perform a fast fourier transform (FFT) on the identified cardiovibration data points to determine a frequency spectrum for the identified cardiovibration data points. The frequency spectrum can decompose the identified cardiovibration data points into components of different frequencies. Additionally, the processor is configured to determine a power spectral density for the determined frequency spectrum. In some implementations, the FFT Power Spectrum of the identified cardiovibration data points may be divide into multiple regions (e.g., three regions). Regions may include the integrated spectrum, low frequency regions below 75 Hz, and/or high frequency regions above 75 Hz.

The processor is configured to determine cardiovibration frequency metrics based on the FFT and FFT Power Spectrum data. In some implementations, cardiovibration frequency metrics may be determined for each region of the FFT power spectrum and/or S1 or S2 heart sounds. In some implementations, cardiovibration frequency metrics may be determined for low frequency regions only.

In some implementations, the cardiovibration frequency metrics may be based on the FFT of the cardiovibration data points and include power amplitude, frequency at maximum power, weighted mean average frequency, and/or frequency standard deviation. In some implementations, the cardiovibration frequency metric may include a peak frequency which corresponds to the frequency at which the power spectral density for the identified cardiovibration data points is the highest. In some implementations, the cardiovibration frequency metrics may include a width which corresponds to the difference between the highest and lowest frequencies present in the identified cardiovibration data points. In some implementations, the cardiovibration frequency metrics includes the mean frequency which corresponds to the average frequency of the identified cardiovibration data points.

In some implementations the cardiovibration frequency metrics may be based on the power frequency spectrum of the cardiovibration data points. For example, the cardiovibration metrics may include one or more of a standard deviation, an entropy measure, or a spectrum bandwidth measure of the frequency spectrum for the identified cardiovibration data points. A spectrum bandwidth may correspond to a difference between the maximum frequency and minimum frequency (including the peak frequency) for which the power spectrum assumes a fixed fraction of total power (e.g. 95% of total power). Cardiovibration frequency metrics may be generated for each heartbeat, each frequency region, each physiological marker, and/or each patient. Cardiovibration frequency metrics may also be averaged over a patient's heartbeats.

In some implementations cardiovibration frequency metrics may be used to determine the risk of having a cardiac event in the future. For example, if scar tissue is present in a patient's heart, the tissue in the heart will be stiffer than healthy heart tissue. Accordingly, lower frequency vibration sounds emitted by a heart with scar tissue will be dampened, such that the average frequency of vibrations would be shifted higher than would be found from healthy tissue. Thus, cardiovibration frequency metrics can be used to determine risk of a patient having a future cardiac event such as cardiac ischemia and/or cardiac arrest.

As illustrated at step 606 of FIG. 9, the processor is configured to provide the one or more cardiovibration frequency metrics for the patient to a trained cardiovibration classifier. A trained cardiovibration classifier may be trained to output risk information or risk assessments for a patient including an indication as to whether a patient is likely to experience a cardiac ischemia and/or sudden cardiac arrest in the future.

In some implementations, the cardiovibration classifier includes a thresholding algorithm. For example, one or more cardiovibration frequency metrics determined for a patient may be compared to thresholds for said cardiovibration frequency metrics. If the determined cardiovibration frequency metrics are above (or below) the indicated thresholds, the patient may be at a greater risk of experiencing a future cardiac event.

In some implementations, the cardiovibration classifier includes one or more of a logistic regression model, support vector machine model, neural network model, a learning survival model, or the like. For example, in some implementations a logistic regression model may include as features the cardiovibration frequency metrics and output a binary classification of the risk associated with the patient having a future cardiac event. In another example, a support vector machine model may be applied to the cardiovibration frequency metrics to output a binary classification of risk (e.g., high-risk or low-risk of having a future cardiac event).

In some implementations, the cardiovibration classifier may be trained at least in part on historical cardiovibration frequency metrics associated with the predetermined physiological marker derived from a plurality of patients. For example, the plurality of patients may include a group of patients providing clinical data including historical cardiovibration frequency metrics for the group of patients. The group of patients may include a first subset of patients that undergoes a future cardiac event and a second subset of patients that does not undergo a future cardiac event. In some implementations, the future cardiac event may require a treatment (e.g., the application of a shock). In some implementations, the historical cardiovibration frequency metrics associated with the predetermined physiological marker are derived from a plurality of patients having a first set of cardiovibration frequency metrics associated with a group of patients who experienced a cardiac arrest and a second set of cardiovibration frequency metrics associated with a group of patients who did not experience a cardiac arrest. Accordingly, the cardiovibration classifier may be trained to distinguish between patients that undergo a future cardiac event and patients that do not undergo a future cardiac event based on their associated cardiovibration frequency metrics.

In an example implementation, a cardiovibration classifier may be trained on heart sound recordings forming a dataset (e.g., 111 total patients). The heart sound recordings may include those taken from a first subset of patients having a sudden cardiac arrest within ten hours of the heart sound recording (e.g., 59 patients who experienced a sudden cardiac arrest of the total of 111 patients), and a second subset of patients who did not experience a sudden cardiac arrest event within ten hours of the heart sound recordings. Data from the dataset of patient cardiovibration data can be analyzed to find statistically significant differences in the mean frequency distributions (e.g., 18.9+/−0.9 Hz and 15.8+/−0.9 Hz for S1, respectively) and in the standard deviation of the mean frequency distributions of the S1 and S2 heart sounds (e.g., 11.5+/−0.4 Hz and 9.5+/−0.6 Hz, respectively) when comparing the subset of data corresponding to patients who go into sudden cardiac arrest and patients who do not. Accordingly, frequency distributions associate with heart sounds can provide indicators for risk associated with sudden cardiac arrest.

As shown at step 608 of FIG. 9, the trained cardiovibration classifier may have been optimized based on one or more predetermined classifier evaluation metrics. For example, during training the cardiovibration classifier may be provided with training data including the historical cardiovibration frequency metrics from a plurality of patients and may be trained to output risk information based on the provided training data. In some implementations, the cardiovibration classifier may be optimized using a predetermined classifier evaluation metrics. For example, predetermined classifier evaluation metrics may include a probability evaluation metric (e.g., Brier score), a concordance index, a specificity index, a sensitivity metric, or an area under the curve metric. In some implementations, optimizing the trained cardiovibration classifier may include adjusting one or more thresholds, weights, or metrics of the cardiovibration classifier to improve one or more of the predetermined classifier metrics. Additionally, the predetermined classifier metrics may provide an indicator for the performance of the cardiovibration classifier.

For example, if the trained cardiovibration classifier provides a binary output, the cardiovibration classifier may be adjusted or tuned to provide sensitivity, specificity and positive predictive values within an expected range. For example, the cardiovibration classifier can be configured to output results having a minimum threshold of sensitivity or within a sensitivity range (e.g., at least at 85% sensitivity, at least at 90% sensitivity, at least at 95% sensitivity, or in a range of 95-100% sensitivity). Similarly, the cardiovibration classifier can be configured to output results having a minimum threshold of specificity or within a specificity range (e.g., at least at 85% specificity, at least 90% specificity, at least 95% specificity, or in a range of 95-100% specificity). In some implementations, the thresholds, weights, and/or metrics of the cardiovibration classifier may be adjusted to achieve the required sensitivity, specificity and positive predictive value. In some implementations, the cardiovibration classifier may provide a probability output or Brier Score and the cardiovibration classifier can be configured to output results having a Brier Score within the required range. For example the classifier may be trained such that Brier Scores between 0 and 0.1, 0 and 0.2, or 0 and 0.3, and the like are achieved.

In some implementations, the trained cardiovibration classifier may be further trained using ECG features. For example, in some implementations the wearable cardiac sensing device may include ECG electrodes that are configured to sense ECG signals from a patient. The ECG signals can then be analyzed to determine one or more ECG features. The ECG features may then be provided to the trained cardiovibration classifier. In some implementations the ECG features provided to the cardiovibration classifier may include historical ECG features that were determined across the plurality of patients. In some implementations, the plurality of patients providing data for the ECG features may be the same as the plurality of patients providing data for the cardiovibration frequency metrics. In some implementations, the plurality of patients providing data for the ECG features may be distinct from the plurality of patients providing data for the cardiovibration frequency metrics. ECG features may include QRS metrics and/or T-wave metrics. Examples of QRS metrics include, but are not limited to, timing of QRS peaks, QRS width, QRS height, R-R length, and the like. Examples of T-wave metrics include T-wave height, timing of T-waves, T-wave alternans or repolarization information, and the like.

Turning back to FIG. 9, at step 610, the processor is configured to output risk information concerning a future cardiac ischemia and/or a sudden cardiac arrest event occurring within a predetermined future period of time. The risk information that is output may be based on applying the trained and optimized cardiovibration classifier on the cardiovibration frequency metrics. For example, the cardiovibration classifier that is trained and optimized based on historical cardiovibration frequency metrics from a plurality of patients may be applied to the cardiovibration frequency metrics determined for a particular patient.

The risk information output by processor may provide a binary indication as to whether a patient is at “high risk” or “low risk” for a future cardiac ischemia or a sudden cardiac arrest event. In some implementations, the risk information output by the processor may provide a classification as to whether the patient is one or a plurality of classes. For example, the classification may indicate whether the patient is of a “high risk class”, “medium risk class”, or “low risk class” for a future cardiac ischemia or sudden cardiac arrest event. In some implementations the processor may output a numerical value as risk information such as a percentage between 0 and 100% or number between 0 and 1, or the like, that indicates probability of having a future cardiac event. A number between 0 and 1 may indicate a probabilistic measure. In some implementations the numerical risk information is considered a risk score. In some implementations, the risk information output by the processor may include a survival function indicative of the probability that the patient will remain event free past a certain time. In some implementations, the risk information output by the processor may include a hazard function indicative of a frequency or rate that a cardiac event will occur, if it has not occurred prior to a certain time.

The risk information output by the processor may be configured to indicate whether the ischemia or the sudden cardiac arrest will occur within a predetermined future period of time. For example, the survival function may be indicative of the probability that the patient will remain event free past the future period of time. Similarly, the hazard function indicative of a frequency or rate that a cardiac event will occur, if it has not occurred during the predetermined future period of time. The predetermined future period of time may be any suitable time period including, without limitation, one month, 14-days, 10-days, 5-days, 3-days, 1, day, 20 hours, 16 hours, 10 hours, 5 hours, 3 hours, 1 hour, or the like. In some implementations the predetermined future period of time may be related to the timespan of the training data (e.g., historical cardiovibration frequency metrics from a plurality of persons) that is provided to the cardiovibration classifier. For example, if the training data is based on three weeks of data then the predetermined future period of time may also be set to three weeks.

In some implementations, the processor may be further configured to output the risk information to a display screen, graphical user interface, electronic health record, clinical interface or the like. For example the risk information may be output to portable gateway 110, technician interfaces 114, and caregiver interfaces 116 as illustrated in FIG. 1. Outputting the risk information may include displaying the risk information on a display screen, generating a medical report including the risk information, initiating an alarm responsive to the risk information, or initiating a therapeutic protocol responsive to the risk information.

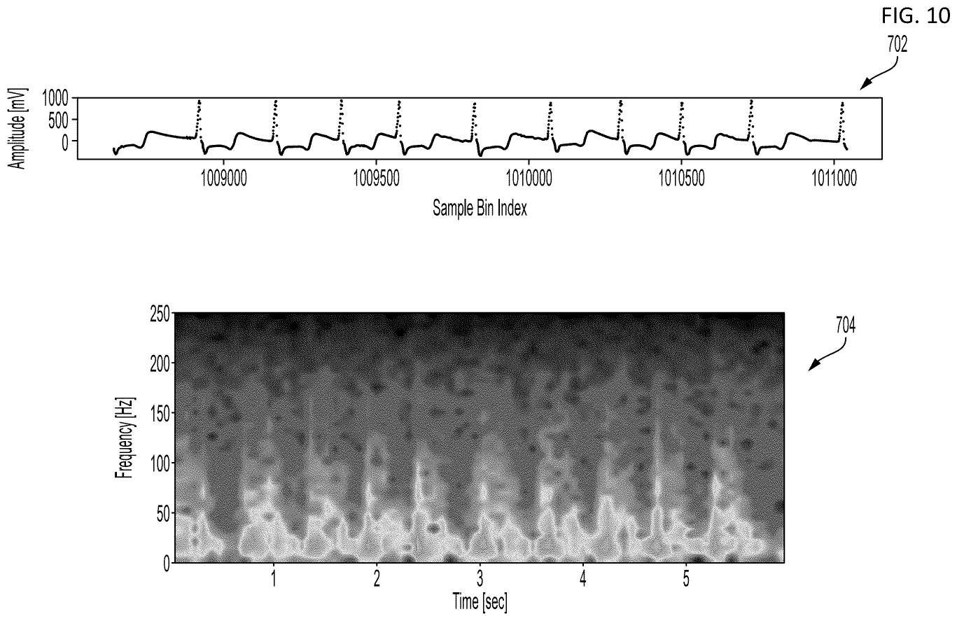

FIG. 10 illustrates cardiovibration data points. A first panel 702 of FIG. 10 provides an illustration of heart sounds or cardiovibration data points identified based on a digitized cardiovibration signal. A second panel 704 provides a spectrogram of the cardiovibration data points illustrated in the first panel 702. The spectrogram illustrates that S1 and S2 sounds in the cardiovibration signal are primarily observed in the low frequency regions (e.g., <75 Hz).

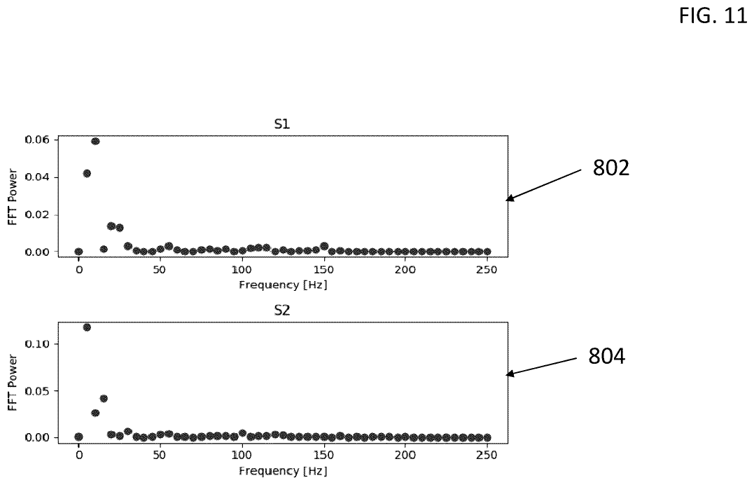

FIG. 11 illustrates power spectrum data for physiological markers of a patient. A first panel 802 of FIG. 11 provides an illustration of the power spectrum associated with S1 Sounds. A second panel 804 of FIG. 11 provides an illustration of the power spectrum associated with a different physiological marker of the patient (e.g., an S2 sound). Both panels in FIG. 11 illustrate the FFT power spectrum from a single heart beat from the S1 and S2 heart sound regions. As discussed above, power spectrum data such as that illustrated in FIG. 11 can be used to determine one or more cardiovibration frequency metrics for the patient associated with the predetermined physiological marker of the patient.

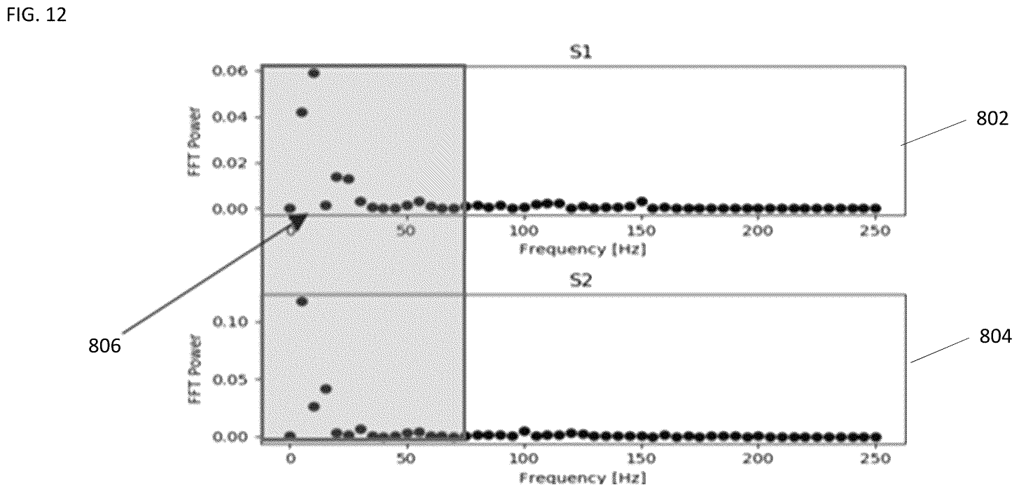

FIG. 12 provides an illustration of cardiovibration frequency metrics and their associated power spectrum data. As discussed above, panels 802, 804 illustrate the FFT power spectrum from a single heart beat from the S1 and S2 heart sound regions, respectively. In FIG. 12, the frequency at the maximum power 806, which is a cardiovibrational frequency metric, is shown.

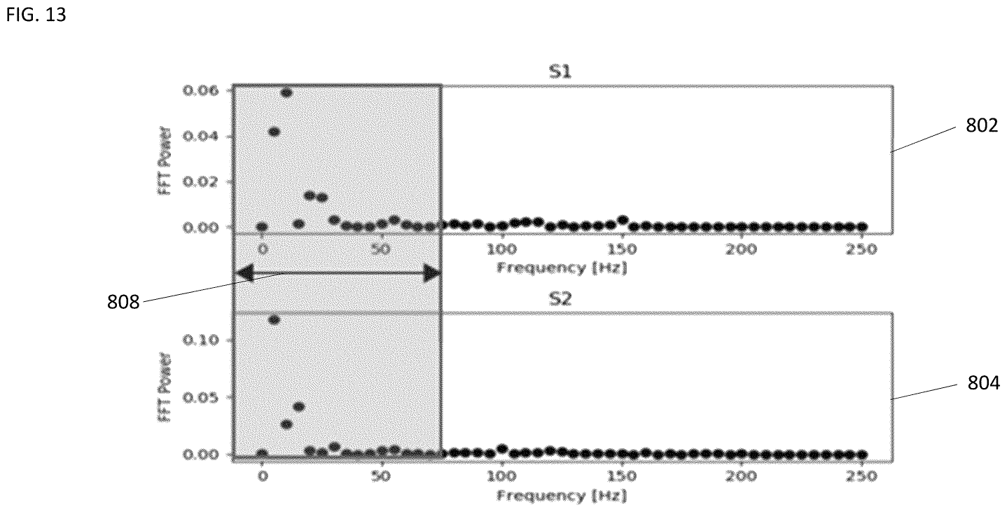

FIG. 13 provides a second illustration of cardiovibration frequency metrics and their associated power spectrum data. As discussed above, panels 802, 804 illustrate the FFT power spectrum from a single heart beat from the S1 and S2 heart sound regions, respectively. In FIG. 13, the mean frequency of the entire signal 808 is shown.

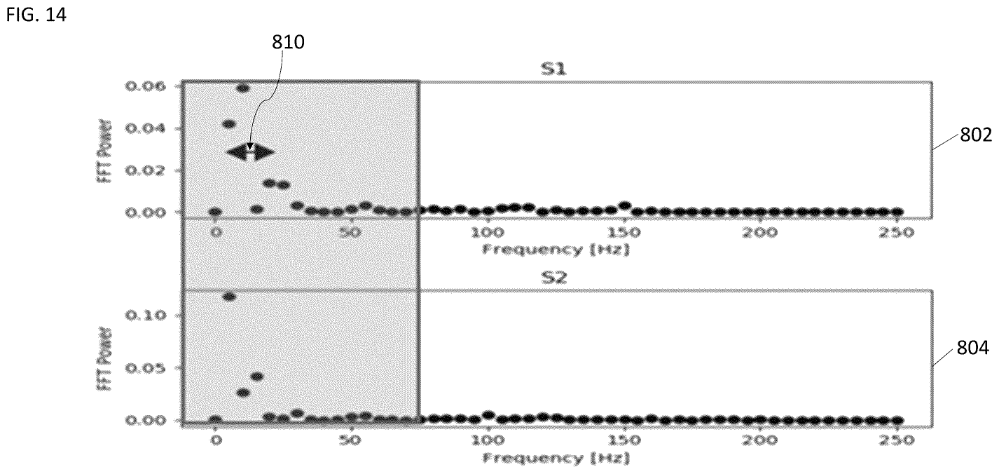

FIG. 14 provides a third illustration of cardiovibrational frequency metrics and their associated power spectrum data. As discussed above, panels 802, 804 illustrate the FFT power spectrum from a single heart beat from the S1 and S2 heart sound regions, respectively. In FIG. 14, the standard deviation and width of frequencies 810 observed at a low frequencies is shown.

FIGS. 12-14 illustrate how cardiovibrational frequency metrics may differ in patients with a higher risk of having a cardiac ischemic event or a sudden cardiac arrest in the future with those who may not. In particular, the frequency at maximum power may be observed to be higher for those who have future cardiac events. Additionally, the mean frequency of the entire signal may be observed to be higher for patients with a higher risk of having a cardiac ischemic event. Further, the width of the peak and standard deviation associated with the cardiovibration frequency is higher for patients with a higher risk of having a cardiac ischemic event.

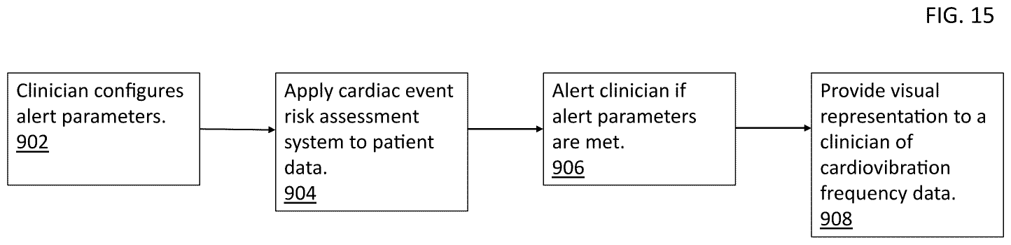

FIG. 15 illustrates a sample process flow 900 for a clinician focused cardiac event risk assessment system that analyzes cardiovibration data to generate and output risk information concerning a future cardiac ischemic or sudden cardiac arrest event occurring in a patient.

As illustrated in FIG. 15, in a first step 902 a physician may configure one or more alert parameters. Alert parameters may include parameters for a frequency index, where the frequency index may be based on a normal spectral heart sound metric. In configuring the alert parameters, the clinician may set alert parameters or ranges based on the frequency index value, averages of the frequency index value, or timescales. For example, the clinician may configure an alert that indicates that they want to be notified if the daily average of either S1 or S2 frequency index exceeds 1.5 for 2 out of 3 days. The clinician may configure alert parameters via a clinical interface. In an example implementation, the frequency index may be based on a single cardiovibration frequency metric (e.g., the entropy of the S1 power spectrum). In other implementations, the frequency index may be based on a combination of a plurality of cardiovibration frequency metrics. In some example implementations, the frequency index may be indexed across a global normal population. In some example implementation, the frequency index may be indexed across patient specific data, such as the patient's baseline during the time period where they first used their device.

In a next step 904, a cardiac event risk assessment system may be applied to patient data to determine that a patient has a risk of having a cardiac ischemia or sudden cardiac arrest in the future using analysis of cardiovibration frequencies as discussed above. Alternatively, the cardiac event risk assessment system may monitor the patient to determine whether the alert parameters set by the clinician have been met. For example, the patient may be monitored and the cardiac event risk assessment system may determine that the patient had a S1 Frequency Index that exceeded 1.5 on days 9 and 10.

In a next step 906, responsive to determining that the alert parameters set by the clinician have been met, the system may generate and transmit an alert or notification to a clinician. The notification may be in the form of an automated phone call, email, electronic health record update, or the like. The notification may provide the clinician with patient information such as their name, address, date of birth, phone number, patient identification number, or other identifying information, as well as a summary of the data gathered from the patient. The summary of the data gathered from the patient may provide an overview of the cardiovibration frequency data that triggered the alert. Additionally, in some implementations, the clinical significance of the observed cardiovibration frequency data may be provided to a clinician.

In a next step 908, a clinician may view event data corresponding to the alert or notification, for example, via a web interface, patient portal, electronic health record, or the like. Accordingly, the clinician may be provided additional information and data to guide clinical decisions for the patient on a clinical interface.

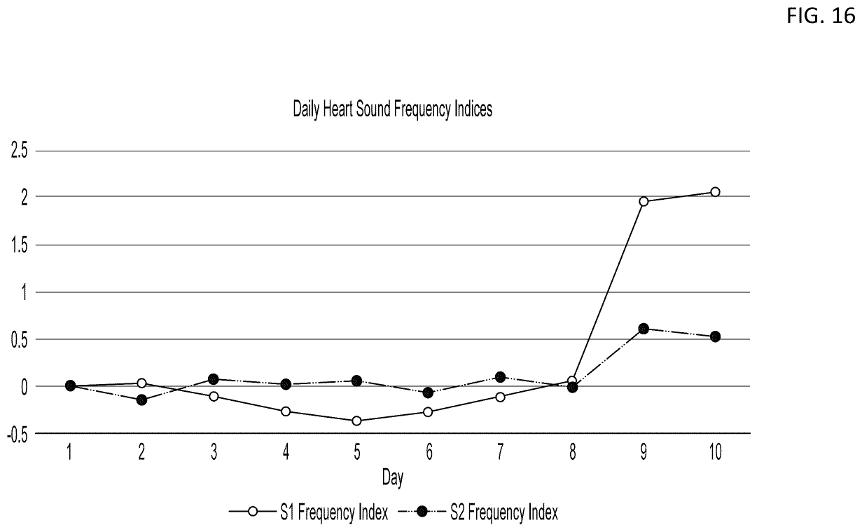

FIG. 16 illustrates an example of how cardiovibration data may be visually displayed to a clinician in step 908 of the process illustrated in FIG. 15. As shown, the cardiovibration data may be characterized by sound frequency indices. Thus, clinicians may be presented with a visualization of the sound frequency indices over an observed time period (e.g., 10 days).

In another example, sudden cardiac arrest, or the complete stoppage of heart activity, is associated with abnormal heartbeats. One example of an abnormal heartbeat are premature ventricular contractions (PVCs), which are extra heartbeats that begin in the heart's ventricle. In particular, PVCs may be generated in runs or sets, where extra heartbeats are in found in a series. In some, PVCs may be a part of a regular pattern that alternates with regular heartbeats. For example, the PVC may be found every second, or third heartbeat, in patterns referred to as n-geminy. In some implementations a cardiac event risk assessment system outputs risk information related to sudden cardiac arrest for a patient by applying a trained machine learning algorithm to data indicative of the PVC count, burden, and/or pattern.

Clinical data indicates that PVCs are more frequent in patients with structural heart disease and hypertension. Additionally, the presence of frequent PVCs demonstrates a significantly higher risk of heart failure, ventricular tachycardia, ventricular fibrillation, and death. Further, PVC-induced cardiomyopathy is recognized as a potential long-term consequence of PVCs. Further, the burden or frequency PVCs have been shown to correlate with the degree of LV dilatation and systolic dysfunction. Accordingly, an analysis of PVC-related data can be used to determine whether the patient has a higher risk for sudden cardiac arrest.

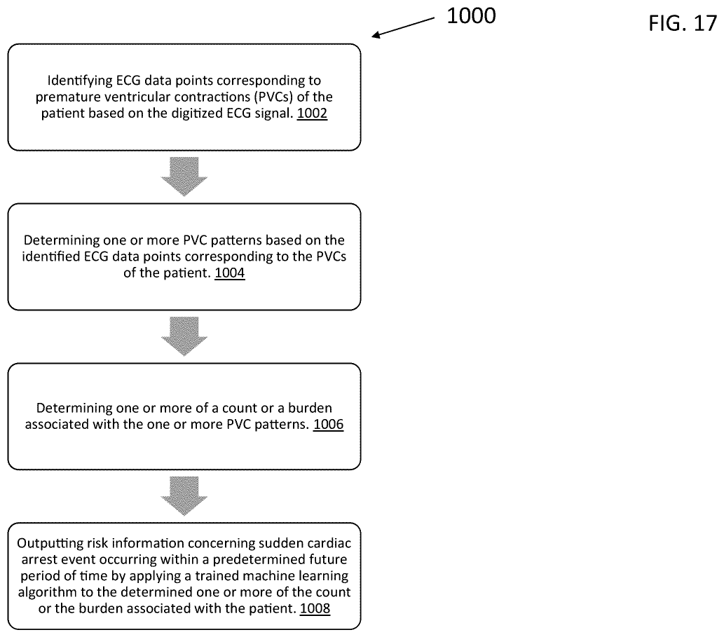

FIG. 17 illustrates a sample process flow in an cardiac event risk assessment system that analyzes data related to PVCs to generate and output risk information concerning a future cardiac event such as sudden cardiac arrest event occurring in a patient.

The sample process 1000 shown in FIG. 17 can be implemented by a processor in communication with the wearable cardiac sensing device 102. In implementations, the processor is part of the wearable cardiac sensing device 102. For example, where the wearable cardiac sensing device 102 includes a cardiac sensing unit 106 and a portable gateway 110, the processor is implemented by the microcontroller 310 of the cardiac sensing unit 106. As another example, the processor may be implemented in the portable gateway 110. As another example, where the wearable cardiac sensing device 102 includes a garment-based sensing device 118, implemented by the processor 518. As another example, the processor is implemented in the remote server 104. As another example, the processor is implemented partially by a processor at the wearable cardiac sensing device 102 and partially by a processor at the remote server 104.

As shown in FIG. 17, the processor is configured to identify ECG data points corresponding to premature ventricular contractions (PVCs) of the patient based on a digitized ECG signal at step 1002. For example, the processor is configured to extract a predetermined timeseries of ECG data points. In implementations, as described above, digitized ECG signals of the patient 100 may be stored in a non-transitory memory of the wearable cardiac sensing device 102. As such, the processor may receive the digitized ECG signals from the wearable cardiac sensing device 102 (e.g., via a transmission of the wearable cardiac sensing device 102) or retrieve the digitized ECG signals from the non-transitory memory of the wearable cardiac sensing device 102. For example, the processor receives or retrieves a predetermined timeseries of ECG data points as the data points forming a segment of the digitized ECG signals, where each of the data points represents an ECG amplitude recorded by the wearable cardiac sensing device 102 at a given time. The digitized ECG signal segments may be pre-segmented (e.g., by the wearable cardiac sensing device 102, such as at the cardiac sensing unit 106, at the portable gateway 110, or at the controller 500 of the garment-based sensing device 118). Alternatively, the digitized ECG signal segments may be segmented by the processor, for instance, at the time the processor receives or retrieves the digitized ECG signals. The segments may be generated according to a predetermined time period (e.g., 60 seconds of ECG data, 90 seconds of ECG data, 120 seconds of ECG data, 5 minutes of ECG data, and/or the like), according to a transmission schedule (e.g., segmented to facilitate transmission of the digitized ECG signals to the remote server 104), according to a symptom event recorded by the wearable cardiac sensing device 102, such that each segment represents a single ECG cycle, and/or the like.

For example, the ECG timeseries can be based on a predetermined sampling frequency and predetermined gain. For example, the predetermined sampling frequency can be between about 100 Hz to about 1000 Hz. For example, the predetermined sampling frequency can be 150 Hz, 240 Hz, 300 Hz, 260 Hz, or 480 Hz. For example, the predetermined gain can be between about 50 and about 1000. For example, the predetermined gain can be 120, 180, 200, 250, 300, or 350. In implementations, ECG can be digitized in a ECG pre-processor configuration (e.g., ECG acquisition circuitry) prior to being processed by the processes described in connection with FIG. 9. For example, the ECG data can be in the form of a single ECG channel, or a plurality of ECG channels (e.g., two or more ECG channels). For example, in implementations, there can be two 16 bit ECG channels that can be acquired and sent to a digital signal processor for signal processing. In examples, a sampling rate can be dynamic or predetermined, e.g., based on a rate determined by the digital signal processor. In examples, an ECG streaming command can be issued by a processor to initiate, pause, or stop the acquisition and streaming of ECG samples from the ECG acquisition circuitry to the digital signal processor. In examples, an acknowledge frame can be provided to allow for synchronization of the ECG data streamed from the ECG acquisition circuitry and the digital signal processor.

In some examples, an electrode falloff signal or indication can be included in the transmission frame, e.g., to allow for the digital signal processor to analyze and determine whether an ECG electrode is deemed to not be making proper contact with skin of the patient. For example, the ECG sensing interface of the processor can incorporate a falloff detection circuit. In this example, a low level AC signal can be applied to the body of the patient and sensed by each ECG electrode circuit. For instance, this fall off signal can be digitized at a 1 Hz interval.

The processor may identify ECG feature data points corresponding to a certain ECG feature from within the digitized ECG signals of the patient 100. Example ECG features may include the P wave, the QRS complex including the QR segment and RS segment, and the T wave. In implementations, the processor may identify the features of an ECG signal by applying a feature extractor. For example, the processor may implement a rhythm classifier stored in a non-transitory computer readable medium (e.g., a memory, a programmable circuit board, a field programmable gate array, an integrated circuit, any combination thereof, and/or the like). The rhythm classifier may include at least one neural network trained based on a historical collection of ECG signal portions with known rhythm information. Among the rhythm information the rhythm classifier is trained to identify may be the certain ECG feature. Additionally, the processor may detect time data corresponding to the certain ECG feature.

As an illustration, in implementations, the processor may use a Pan-Tompkins-based QRS detector. The processor may first filter the ECG feature data points. For instance, the processor may receive or retrieve raw ECG data sampled at, say, 250 Hz. The processor may then remove baseline wander, high frequency noise, and 50/60 Hz interference. This filtering may be represented mathematically by denoting the raw ECG data as x and representing the process as:

$${y}_{1} = b p f * x$$

In this equation, bpf represents band pass filtering, * stands for convolution, and y1 represents the filtered signal. The processor may find the derivative of the filtered y1 signal shown above square the result, which may be represented as:

$${y}_{2} = {( \frac{d {y}_{1}}{d t} )}^{2}$$

Next, the processor may apply a moving average to the y2 result, which may be further represented mathematically by:

$${y}_{3} = MovingAverageFilter * {y}_{2}$$

The processor then may apply an adaptive power threshold to locate the QRS complexes in the y3 signal. However, as other illustrations, the processor may use a Hilbert transform process or a phasor transform process to identify the ECG feature data points corresponding to the certain ECG feature. In implementations, the rhythm classifier may determine a confidence score associated with the detected ECG feature data points (e.g., output a confidence score associated with the probability that the certain ECG feature data points were identified correctly).

The processor is further configured to identify ECG data points corresponding to premature ventricular contractions (PVCs). PVCs may be identified by analyzing the morphological space associated with the ECG feature data points. The processor is further configured to determine timestamps associated with PVCs. Any one of a number of suitable methods may be used to identify ECG data points associated with PVCs including morphological space analysis, wavelet transform, automated diagnostic methods, deep neural networks, state vector machines, support vector machines, Gaussian process methods, and the like.

At step 1004 of FIG. 17, the processor is further configured to determine one or more PVC patterns based on the identified ECG data points corresponding to the PVCs of the patient. PVC patterns can include a PVC run, which is indicative of a number of consecutive PVCs. For example, a PVC run may be a singlet, where a heartbeat having a PVC is spaced apart from other heartbeats having a PVC. A PVC run may be a couplet where there are two heartbeats with PVCs in consecutive heartbeats. A PVC run may be a triplet where there are three heartbeats with PVCs consecutively. A PVC run may be a quadruplet where there are four heartbeats with PVCs. A PVC run may be a non-sustained ventricular tachycardia (NSVT) where there are five or more consecutive heartbeats with PVCs.

PVC patterns can also include n-geminy which is indicative of the interval between PVCs. In some patients, PVCs occur in repeating patterns. For example, N-geminy may include bigeminy, trigeminy, quadrageminy, or 5-geminy. Bigeminy is when every other heartbeat is a PVC. Trigeminy is when every third heartbeat is a PVC. Quadrigeminy is when every fourth beat is a PVC. 5-geminy is when every fifth beat is a PVC.

The processor may determine the PVC pattern based on the identified ECG data points corresponding to the PVCs of the patient by analyzing the timestamps corresponding to the PVCs for a patient. For example, when ECG data points identified as corresponding to the PVCs are located in two adjacent heartbeats, it may be a part of a PVC couplet. In another example, if the ECG data points identified as corresponding to PVCs are spaced by one heartbeat, then the processor may determine that the PVC pattern is bigeminy.

Turning back to FIG. 17, at step 1006 the processor is configured to determine counts and/or burdens associated with the determined PVC patterns. For example, in some embodiments the burden associated with the determined one or more PVC patterns includes a count associated with the one or more PVC patterns over the total time of the digitized ECG signal. In some implementations, burdens may be calculated over a minute, 30-minutes, an hour, 5-hours, and the like. In some implementations, the processor may determine a Lown score associated with the count or burden. The Lown score may be a measure of the grade of seriousness for PVC classification of an ECG sequence. For example, the severity of the observed PVC pattern can be classified as having Lown scores between Grade 0 and Grade V as shown in Table 1.

TABLE 1
Lown Score Definitions for PVC Classification

| | |
|---|---|
| Grade 0 | No ventricular extrasystoles/ectopics |
| Grade I | Unifocal and infrequent PVCs; <30 PVCs per hour |
| Grade II | Unifocal and frequent PVCs; >=30 PVCs per hour |
| Grade III | Multifocal |
| Grade IVA | 2 consecutive beats (couplets) |
| Grade IVB | >=3 consecutive beats (salvos) |
| Grade V | “R on T” phenomenon |

At step 1008 of FIG. 17, the processor is configured to output risk information concerning a sudden cardiac arrest event occurring within a predetermined future period of time by applying a trained machine learning algorithm to the determined one or more of the count or the burden associated with the patient. In some implementations, the machine learning algorithm may have been trained at least in part on historical PVC pattern data derived from a plurality of patients. For example, the historical PVC pattern data may be derived from a plurality of patients including a first set of PVC pattern data associated with a group of patients who experienced a sudden cardiac arrest event and a second set of PVC pattern data associated with a group of patients who did not experience a sudden cardiac arrest event.

In some implementations the machine learning algorithm may include gradient boosting classifiers that are trained to determine a binary risk for the patient having a future cardiac event based on the PVC burdens and/or counts. In some embodiments, the gradient boosting classifiers may be applied to the determined Lown Scores for the patient. In some implementations, the gradient boosting classifier may include hyperparameters tuned using a grid search. In some implementations, the gradient boosting classifier may be applied to a combination of Lown Scores and other metrics. In some implementations, the gradient boosting classifier may be applied to metrics distinct from Lown Scores.

In some implementations, the inclusion of PVC burdens and/or counts into the training of a machine learning based algorithm may result in a classifier that performs with greater specificity and/or sensitivity. For example, a model including PVC burdens and/or counts in its training may provide increased performance (e.g., area under the curve, and sensitivity at specificity, and the like) when compared to a model that does not include PVC burdens and/or counts.

In some implementations, the machine learning algorithm may include logistic regression, naïve Bayes, random forest, gradient boosting, neural networks, learned survival models. The logistic regression, naïve Bayes, random forest, gradient boosting, neural networks, learned survival models may be trained on data including the historical PVC pattern data. In some embodiments, the trained machine learning algorithm may be configured to output risk information. Examples of machine learning algorithms include, but are not limited to, a logistic regression model, support vector machine model, neural network model, a learning survival model, and the like.

The processor is configured to output risk information based on applying the trained machine learning algorithm(s) to PVC burdens, PVC counts and/or Lown scores.

The risk information output by processor may provide a binary indication as to whether a patient is at “high risk” or “low risk” for a future sudden cardiac arrest event. In some implementations, the risk information output by the processor may provide a classification as to whether the patient is one or a plurality of classes. For example, the classification may indicate whether the patient is of a “high risk class”, “medium risk class”, or “low risk class” for a future sudden cardiac arrest event. In some implementations the processor may output a numerical value as risk information such as a percentage between 0 and 100% or number between 0 and 1, or the like, that indicates probability of having a future cardiac event. A number between 0 and 1 may indicate a probabilistic measure. In some implementations the numerical risk information is considered a risk score. In some implementations, the risk information output by the processor may include a survival function indicative of the probability that the patient will remain event free past a certain time. In some implementations, the risk information output by the processor may include a hazard function indicative of a frequency or rate that a cardiac event will occur, if it has not occurred prior to a certain time.

The risk information output by the processor may be configured to indicate whether the sudden cardiac arrest will occur within a predetermined future period of time. For example, the survival function may be indicative of the probability that the patient will remain event free past the future period of time. Similarly, the hazard function indicative of a frequency or rate that a cardiac event will occur, if it has not occurred during the predetermined future period of time. The predetermined future period of time may be any suitable time period including, without limitation, one month, 14-days, 10-days, 5-days, 3-days, 1, day, 20 hours, 16 hours, 10 hours, 5 hours, 3 hours, 1 hour, or the like. In some implementations the predetermined future period of time may be related to the timespan of the training data that is provided to the trained machine learning algorithm. For example, if the training data is based on three weeks of data then the predetermined future period of time may also be set to three weeks.

In some implementations, the processor is further configured to output the risk information to a display screen, graphical user interface, electronic health record, clinical interface or the like. For example the risk information may be output to portable gateway 110, technician interfaces 114, and caregiver interfaces 116 as illustrated in FIG. 1. Outputting the risk information may include displaying the risk information on a display screen, generating a medical report including the risk information, initiating an alarm responsive to the risk information, or initiating a therapeutic protocol responsive to the risk information, analogous to what was described above.

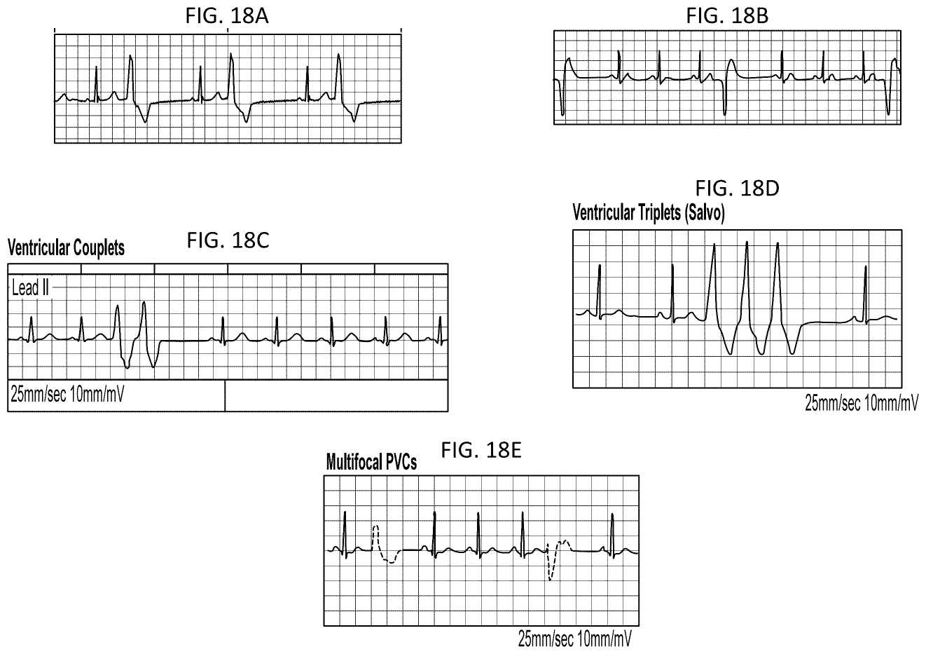

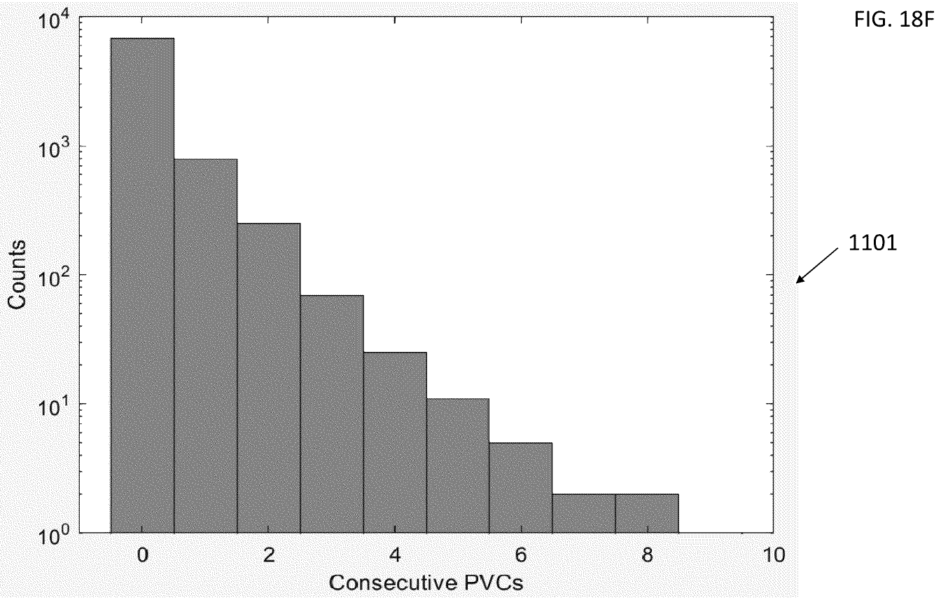

FIGS. 18A-18F provide illustrations of PVC patterns as determined for a patient. FIG. 18A provides an illustration of data points corresponding to a bigeminy pattern, where every other heartbeat is a PVC. FIG. 18B provides an illustration of data points corresponding to a quadrageminy pattern, where every fourth beat is a PVC. FIG. 18C provides an illustration of data points corresponding to couplets, and particularly ventricular couplets, where there are two heartbeats with PVCs in consecutive heartbeats. FIG. 18D provides an illustration of data points corresponding to a triplets, and particularly ventricular triplets, where there are three heartbeats with PVCs consecutively. FIG. 18E provides an illustration of multi-focal PVCS, or PVCs that originate from multiple regions.

FIG. 18F provides an illustration of PVC counts and patterns determined for a patient. As shown in the histogram 1101 PVC patterns such as consecutive PVCs may be determined from the ECG data points corresponding to PVCs. The patient data presented in FIG. 18F shows that the patient has a high number of normal heartbeats (without consecutive PVCs) and approximately 10³ single PVCs, and the like. As shown, the patient experiences far fewer heartbeats with higher numbers of consecutive PVCs than normal heartbeats without PVCs or with smaller numbers of consecutive PVCs.

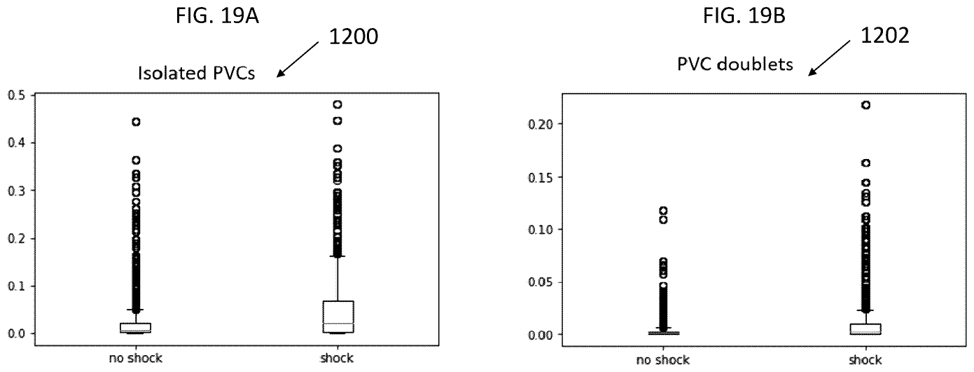

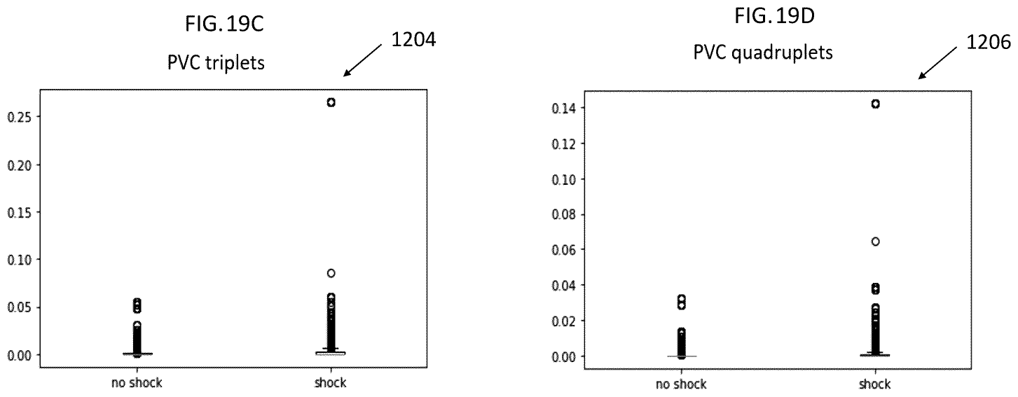

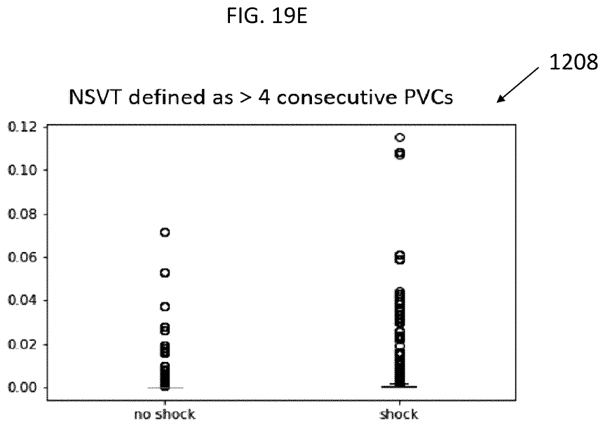

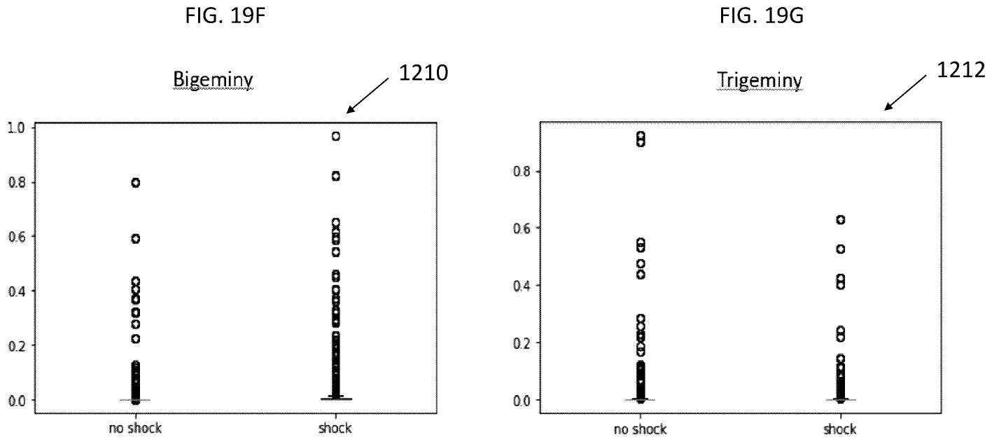

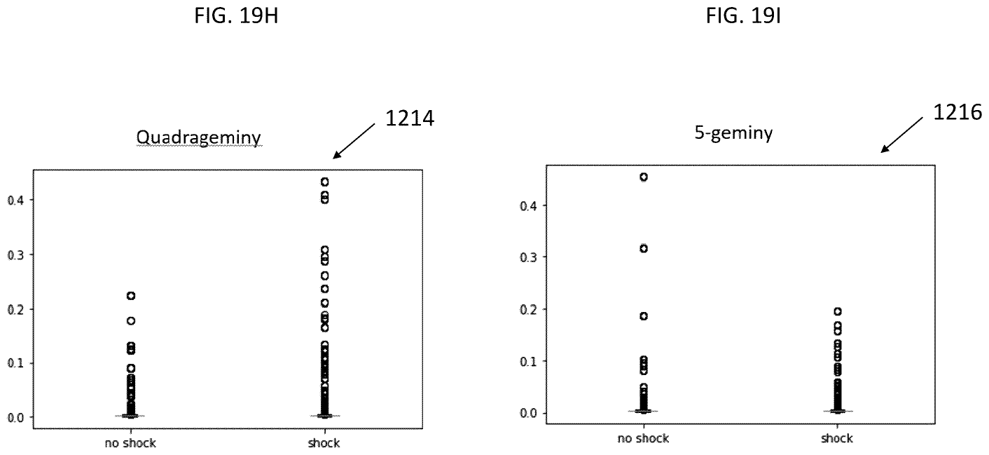

FIGS. 19A-19I provide illustrations of example training data that includes historical PVC/n-geminy pattern data that may be provided to a machine learning algorithm that is configured to determine risk information related to whether a patient will have a future cardiac event based on their PVC/n-geminy pattern data. As shown in box plots of FIGS. 19A-19I, example training data may include historical PVC/n-geminy pattern data for two categories of patients: (1) those receiving a shock, and therefore undergoing a cardiac event (e.g., sudden cardiac arrest) and (2) those not receiving a shock. FIGS. 19A-19I illustrate PVC/n-geminy metrics for both categories of patients. In implementations, the boxes depict an interquartile range, e.g., between 25^{th} and 75^{th} percentile. Further, the line within the boxes depict a median value for the corresponding data for both categories of patients. The circles depict statistical outliers in the data and are shown for both categories of patients.

In particular, FIGS. 19A-19E illustrate box plots depicting burdens associated with PVC runs in the two categories of patients in the example training data. In these FIGS. 19A-19E, the circles represent patient records within the example training data, e.g. outliers within a dataset. Further, the y-axis describes a PVC run burden value associated with the corresponding patient record. In one example scenario, the PVC run burden value can be calculated by summing all the beats matching a corresponding description of the PVC run and dividing by the total number of beats. For example, such descriptions of PVC runs include isolated PVC for FIG. 19A, PVC doublets for FIG. 19B, PCF triplets for FIG. 19C, PVC quadruplets for FIG. 19D, and a non-sustained ventricular tachycardia (NSVT) burden, e.g., defined, in this example scenario, as four consecutive PVCs. For example, as shown in FIG. 19A, patients receiving a shock and therefore undergoing a cardiac event had a greater PVC burden value of isolated PVCs 1200. For example, a median value for the isolated PVCs is higher for the shock category than the no shock category. Further, the 27th percentile end of the box for the shock category is higher than the no shock category. As such, the underlying data can be used as one form of training input to a risk assessment machine learning model as described herein. Similarly, as shown in FIG. 19B, in a chart illustrating burdens associated with PVC doublets 1202, patients receiving a shock and therefore undergoing a cardiac event had a greater PVC burden value of PVC doublets. In FIG. 19C a chart illustrating burdens associated with PVC triplets 1204 is shown. As illustrated, patients receiving a shock and therefore undergoing a cardiac event had a greater PVC burden value of PVC triplets. In FIG. 19D a chart illustrating burdens associated with PVC quadruplets 1206 is shown. As illustrated, patients receiving a shock and therefore undergoing a cardiac event had a greater PVC burden value of PVC quadruplets. In FIG. 19E a chart illustrating burdens associated with non-sustained ventricular tachychardia (NSVT) or greater than four consecutive PVCs 1208 is shown. As illustrated, patients receiving a shock and therefore undergoing a cardiac event had a greater PVC burden value of NSVT.

In particular, FIGS. 19F-19I illustrate box plots depicting burdens associated with n-geminy in the two categories of patients in the example training data. In these FIGS. 19F-19I, similar to the above, the circles represent patient records within the example training data, e.g. outliers within a dataset. Further, the y-axis describes an n-geminy burden value associated with the corresponding patient record. In one example scenario, the n-geminy burden value can be calculated by summing all the beats matching a corresponding description of the n-geminy and dividing by the total number of beats. For example, as shown in the chart 1210 of FIG. 19F, patients with higher n-geminy burden values corresponding to a bigeminy pattern were associated with undergoing a cardiac event. Similarly, as shown in chart 1212 of FIG. 19G, patients with higher n-geminy burden values corresponding to a trigeminy pattern were associated with undergoing a cardiac event. Further, as shown in chart 1214 of FIG. 19H, patients with higher n-geminy burden values corresponding to a quadrageminy pattern were associated with undergoing a cardiac event. And as shown in chart 1216 of FIG. 19I, patients with higher n-geminy burden values corresponding to a 5-geminy pattern were associated with undergoing a cardiac event.

Accordingly, the example training data visualized in FIGS. 19A-19I demonstrate that a PVC pattern data indicating a higher presence of isolated PVCs, PVC doublets, PVC triplets, PVC quadruplets, NSVT Burdens, Bigeminy, Trigeminy, Quadrageminy, and/or 5-Geminy may be associated with a higher risk of having a sudden cardiac arrest event. In some embodiments the PVC pattern data having the most predictive power may depend on the target outcome. For example, when the risk assessment system is identifying risk associated with heart failure the PVC pattern data most associated with risk associated with heart failure may be different than the PVC pattern data most associated with sudden cardiac arrest. Accordingly, the PVC pattern data metric having the greatest predictive power may vary depending on the type of risk assessed by the risk assessment system.

As discussed above, sudden cardiac arrest is associated with abnormal heartbeats such as premature ventricular contractions (PVCs). However, PVCs may originate from more than one focal point, or from different locations in the ventricles, indicating that multiple areas of the ventricles may have altered myocardial contractility. Accordingly, patients with PVCs that originate from more than one focal point can be associated with a greater risk of having a sudden cardiac arrest event.

Additionally, patients with notches in their QRS patterns are also associated with a greater risk of sudden cardiac arrest. Notches in QRS patterns may indicate abnormal contraction due to scarring of heart tissue and the like.

Accordingly, in some implementation, a cardiac event risk assessment system may output risk information related to sudden cardiac arrest for a patient by applying a clustering algorithm that determines whether the patient has PVCs that originate from multiple foci. Optionally, this risk assessment system can be coupled with a risk assessment system that analyzes notches in QRS patterns.

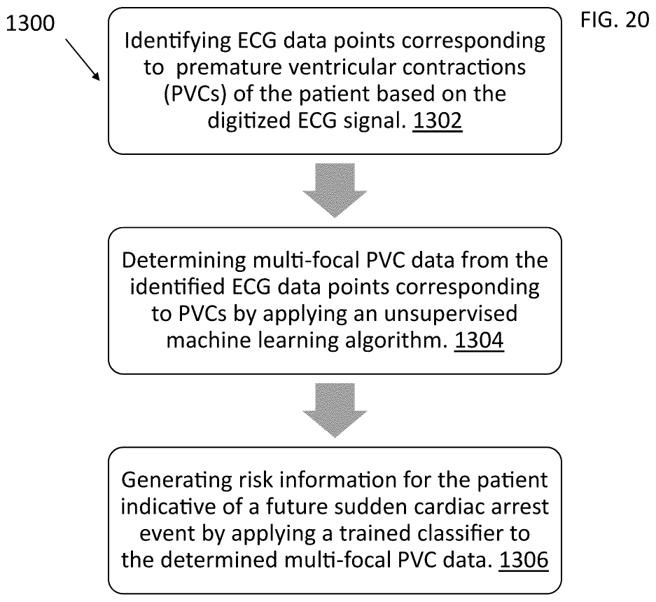

FIG. 20 illustrates a sample process flow in a cardiac event risk assessment system that analyzes data related to PVC focal points to generate and output risk information concerning a future cardiac event such as a sudden cardiac arrest event occurring in a patient.

The sample process 1300 shown in FIG. 20 can be implemented by a processor in communication with the wearable cardiac sensing device 102. In implementations, the processor is part of the wearable cardiac sensing device 102. For example, where the wearable cardiac sensing device 102 includes a cardiac sensing unit 106 and a portable gateway 110, the processor is implemented by the microcontroller 310 of the cardiac sensing unit 106. As another example, the processor is implemented in the portable gateway 110. As another example, where the wearable cardiac sensing device 102 includes a garment-based sensing device 118, the processor is implemented by the processor 518. As another example, the processor is implemented in the remote server 104. As another example, the processor is implemented partially by a processor at the wearable cardiac sensing device 102 and partially by a processor at the remote server 104.

As shown in FIG. 20, the processor is configured to identify ECG data points corresponding to premature ventricular contractions (PVCs) of the patient based on a digitized ECG signal at step 1302. For example, the processor is configured to extract a predetermined timeseries of ECG data points. In implementations, as described above, digitized ECG signals of the patient 100 are stored in a non-transitory memory of the wearable cardiac sensing device 102. As such, the processor may receive the digitized ECG signals from the wearable cardiac sensing device 102 (e.g., via a transmission of the wearable cardiac sensing device 102) or retrieve the digitized ECG signals from the non-transitory memory of the wearable cardiac sensing device 102. For example, the processor may receive or retrieve a predetermined timeseries of ECG data points as the data points forming a segment of the digitized ECG signals, where each of the data points represents an ECG amplitude recorded by the wearable cardiac sensing device 102 at a given time. The digitized ECG signal segments may be pre-segmented (e.g., by the wearable cardiac sensing device 102, such as at the cardiac sensing unit 106, at the portable gateway 110, or at the controller 500 of the garment-based sensing device 118). Alternatively, the digitized ECG signal segments may be segmented by the processor, for instance, at the time the processor receives or retrieves the digitized ECG signals. The segments may be generated according to a predetermined time period (e.g., 60 seconds of ECG data, 90 seconds of ECG data, 120 seconds of ECG data, 5 minutes of ECG data, and/or the like), according to a transmission schedule (e.g., segmented to facilitate transmission of the digitized ECG signals to the remote server 104), according to a symptom event recorded by the wearable cardiac sensing device 102, such that each segment represents a single ECG cycle, and/or the like.

As discussed above, in some examples, the ECG timeseries can be based on a predetermined sampling frequency and predetermined gain. For example, the predetermined sampling frequency can be between about 100 Hz to about 1000 Hz. For example, the predetermined sampling frequency can be 150 Hz, 240 Hz, 300 Hz, 260 Hz, or 480 Hz. For example, the predetermined gain can be between about 50 and about 1000. For example, the predetermined gain can be 120, 180, 200, 250, 300, or 350. In implementations, ECG can be digitized in a ECG pre-processor configuration (e.g., ECG acquisition circuitry) prior to being processed by the processes described in connection with FIG. 20. For example, the ECG data can be in the form of a single ECG channel, or a plurality of ECG channels (e.g., two or more ECG channels). For example, in implementations, there can be two 16 bit ECG channels that can be acquired and sent to a digital signal processor for signal processing. In examples, a sampling rate can be dynamic or predetermined, e.g., based on a rate determined by the digital signal processor. In examples, an ECG streaming command can be issued by a processor to initiate, pause, or stop the acquisition and streaming of ECG samples from the ECG acquisition circuitry to the digital signal processor. In examples, an acknowledge frame can be provided to allow for synchronization of the ECG data streamed from the ECG acquisition circuitry and the digital signal processor.

As also discussed above, in some examples, an electrode falloff signal or indication can be included in the transmission frame, e.g., to allow for the digital signal processor to analyze and determine whether an ECG electrode is deemed to not be making proper contact with skin of the patient. For example, the ECG sensing interface of the processor can incorporate a falloff detection circuit. In this example, a low level AC signal can be applied to the body of the patient and sensed by each ECG electrode circuit. For instance, this fall off signal can be digitized at a 1 Hz interval.

Analogous to the process described above, in some implementations the processor may identify ECG feature data points corresponding to a certain ECG feature from within the digitized ECG signals of the patient 100. Example ECG features may include the P wave, the QRS complex including the QR segment and RS segment, and the T wave. In implementations, the processor may identify the features of an ECG signal by applying a feature extractor. For example, the processor may implement a rhythm classifier stored in a non-transitory computer readable medium (e.g., a memory, a programmable circuit board, a field programmable gate array, an integrated circuit, any combination thereof, and/or the like). The rhythm classifier may include at least one neural network trained based on a historical collection of ECG signal portions with known rhythm information. Among the rhythm information the rhythm classifier is trained to identify may be the certain ECG feature. Additionally, the processor may detect time data corresponding to the certain ECG feature.

As an illustration, in implementations, the processor may use a Pan-Tompkins-based QRS detector, Hibert transform process, or phasor transform process, rhythm classifier and the like (discussed above).

The processor may be further configured to identify ECG data points corresponding to premature ventricular contractions (PVCs). PVCs may be identified by analyzing the morphological space associated with the ECG feature data points. The processor may be further configured to determine timestamps associated with PVCs. Any one of a number of suitable methods may be used to identify ECG data points associated with PVCs including morphological space analysis, wavelet transform, automated diagnostic methods, deep neural networks, state vector machines, support vector machines, Gaussian process methods, and the like.

Turning back to FIG. 20, at step 1304, the processor is configured to determine multi-focal PVC data from the identified ECG data points corresponding to PVCs. For example, at step 1304 the processor can apply an unsupervised machine learning algorithm that is trained to determine multiple focal points when provided with a set of PVCs. In some embodiments, the unsupervised machine learning algorithm may include an unsupervised clustering algorithm.

In some implementations the unsupervised clustering algorithm may be a K-means unsupervised clustering algorithm. The unsupervised clustering algorithm may be applied to patient PVC characteristics data for each identified PVC in the identified ECG data points that correspond to one or more PVCs of the patient. Patient PVC characteristics data may include the R-R duration for the PVC, a R-R duration for a previously identified PVC, a local prematurity index, a beat type, QRS duration of the identified PVC, a morphology score for the identified PVC, an area corresponding to a R-S segment for the identified PVC, a ratio between a Q-R segment for the identified PVC to a R-S segment for the identified PVC, and the like.

In some implementations, the unsupervised clustering algorithm may be a 2-means unsupervised clustering algorithm. In such an implementation, patient PVC characteristics data may include a morphology score for the identified PVC, and a QRS width for the identified PVC.

After the processor has determined patient PVC characteristics data from the identified ECG data points corresponding to the PVCs, the processor can determine multi-focal PVC data by generating a proposed clustering of PVCs, and then generating a final clustering of PVCs. For example, the processor may generate a proposed clustering of PVCs by applying the unsupervised clustering algorithm to the patient PVC characteristics data. The processor may generate a final clustering of PVCs by one or more of maximizing a silhouette coefficient for the proposed clustering of PVCs and/or maximizing a gap score for PVCs in each of the proposed clusters.

The multi-focal PVC data determined by the processor may indicate a number of focal points corresponding to the identified ECG data points corresponding to PVCs. Alternatively, or additionally, the multi-focal PVC data may provide an indicator of the presence or absence of multiple focal points within a set of PVCs.

As shown in FIG. 20, at step 1306, the processor is also configured to generate risk information for the patient indicative of a future sudden cardiac arrest by applying a trained classifier to the determined multi-focal PVC data. The classifier may include one or more of a logistic regression model, support vector machine model, neural network model, learning survival model, and the like. In some implementations the classifier for determining risk information may have been trained on a historical data set of multi-focal PVC data. For example, the historical data set of multi-focal PVC data may have been derived from a plurality of patients including a first set of patients who experienced a sudden cardiac arrest and a second set of patients who did not experience a sudden cardiac arrest event. In some implementations, training data including the historical data set of multi-focal PVC data may include labeled data labeled by experts. Further, in some implementations the classifier may be trained until it achieves performance metrics set by a user.

In an example implementation, a the historical data set of multi-focal PVC data included labeled data from patients wearing a cardiac monitor. Data included one hour of ECG data in the hour leading up to the patient experiencing a sudden cardiac arrest. In a data set composed of ten patients, a first subset of five patients had a single PVC morphology and a second subset of five patients had multiple PVC morphologies. Labeled data included labels indicating PVC morphology. In some implementations, labeling may be performed by trained ECG technicians, automated algorithms, and the like. In some embodiments, an unsupervised classifier may be used on the multi-focal PVC data,
such that a ROC-AUC or accuracy score is not reported. In the example implementation, the average number of optimal number of clusters determined by using the Silhouette score method is 2.4 for patients with multiple PVC morphologies and 1.2 for patients with a single PVC morphology after excluding the cluster consisting of normal beats.

In an example embodiment, the historical data may include ECG data from patients who are known to have experienced a sudden cardiac arrest event.

The risk information output by processor may provide a binary indication as to whether a patient is at “high risk” or “low risk” for a future sudden cardiac arrest event. In some implementations, the risk information output by the processor may provide a classification as to whether the patient is one or a plurality of classes. For example, the classification may indicate whether the patient is of a “high risk class”, “medium risk class”, or “low risk class” for a future sudden cardiac arrest event. In some implementations the processor may output a numerical value as risk information such as a percentage between 0 and 100% or number between 0 and 1, or the like, that indicates probability of having a future cardiac event. A number between 0 and 1 may indicate a probabilistic measure. In some implementations the numerical risk information is considered a risk score. In some implementations, the risk information output by the processor may include a survival function indicative of the probability that the patient will remain event free past a certain time. In some implementations, the risk information output by the processor may include a hazard function indicative of a frequency or rate that a cardiac event will occur, if it has not occurred prior to a certain time.

The risk information output by the processor may be configured to indicate whether the sudden cardiac arrest will occur within a predetermined future period of time. For example, the survival function may be indicative of the probability that the patient will remain event free past the future period of time. Similarly, the hazard function indicative of a frequency or rate that a cardiac event will occur, if it has not occurred during the predetermined future period of time. The predetermined future period of time may be any suitable time period including, without limitation, one month, 14-days, 10-days, 5-days, 3-days, 1, day, 20 hours, 16 hours, 10 hours, 5 hours, 3 hours, 1 hour, or the like. In some implementations the predetermined future period of time may be related to the timespan of the training data that is provided to the trained machine learning algorithm. For example, if the training data is based on three weeks of data then the predetermined future period of time may also be set to three weeks.

In some implementations, the processor may be further configured to output the risk information to a display screen, graphical user interface, electronic health record, clinical interface or the like. For example the risk information may be output to portable gateway 110, technician interfaces 114, and caregiver interfaces 116 as illustrated in FIG. 1. Outputting the risk information may include displaying the risk information on a display screen, generating a medical report including the risk information, initiating an alarm responsive to the risk information, or initiating a therapeutic protocol responsive to the risk information, analogous to what was described above.

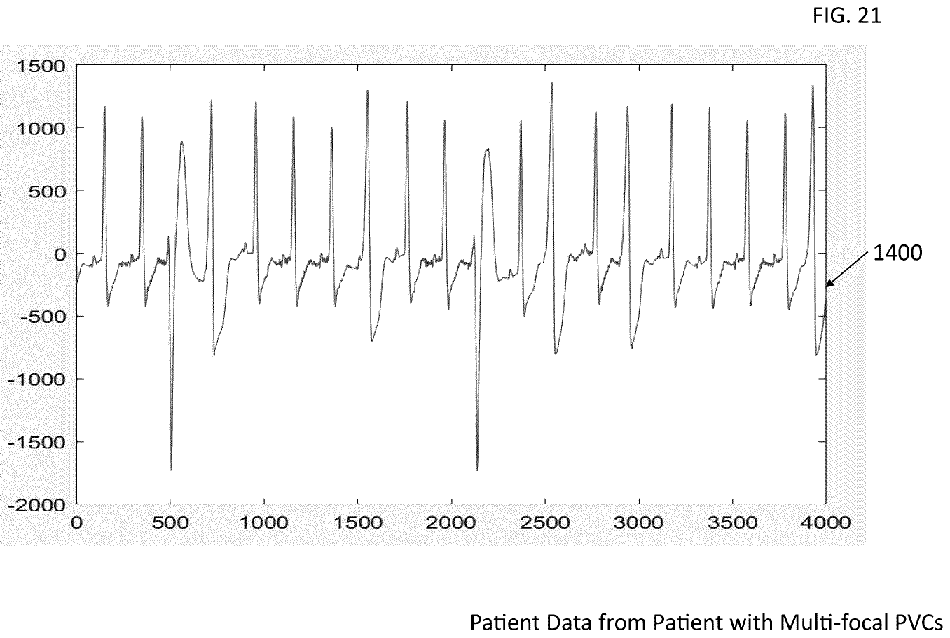

FIG. 21 illustrates a digitized ECG signal corresponding to a patient having multi-focal PVCs. As shown, the ECG signal includes PVCs with different morphologies. Signal 1400 is an example of the signal that may be used to identify ECG data points corresponding to PVCs in step 1302 of process 1300 illustrated in FIG. 20.

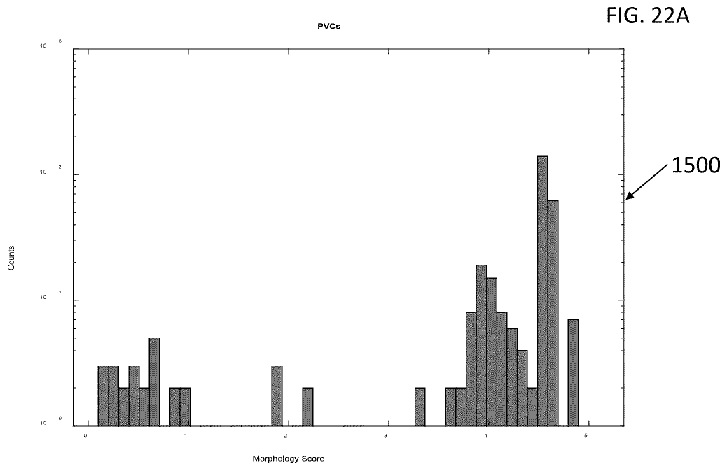

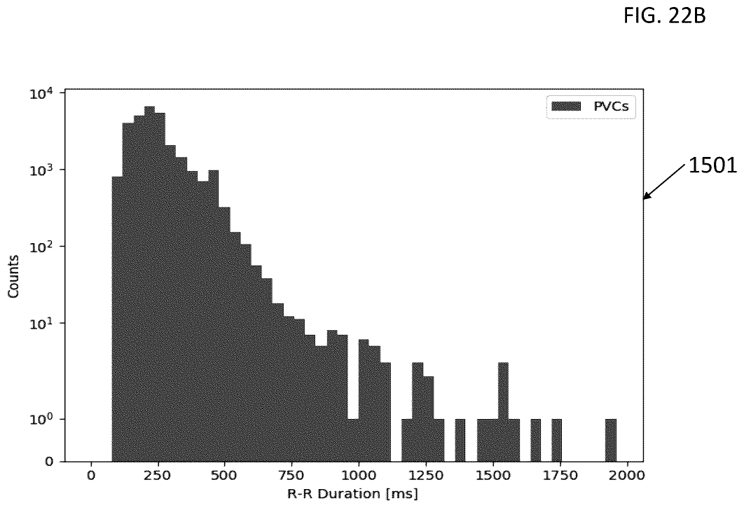

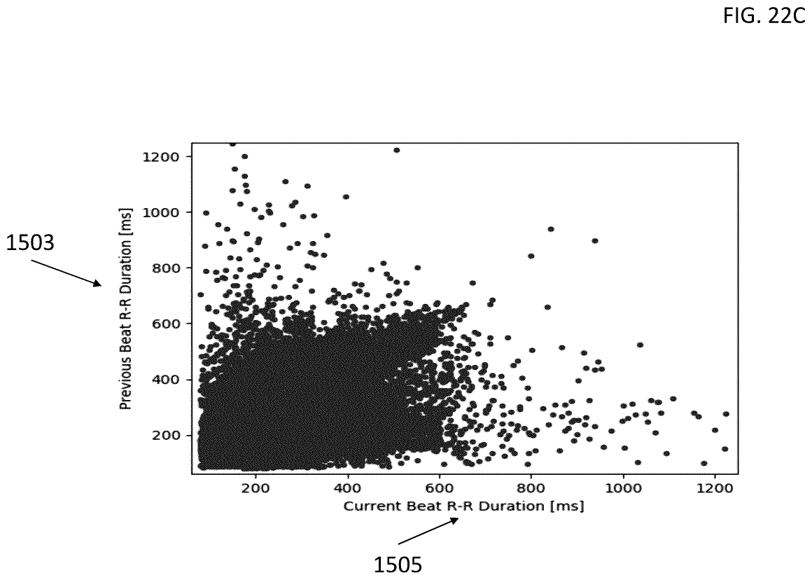

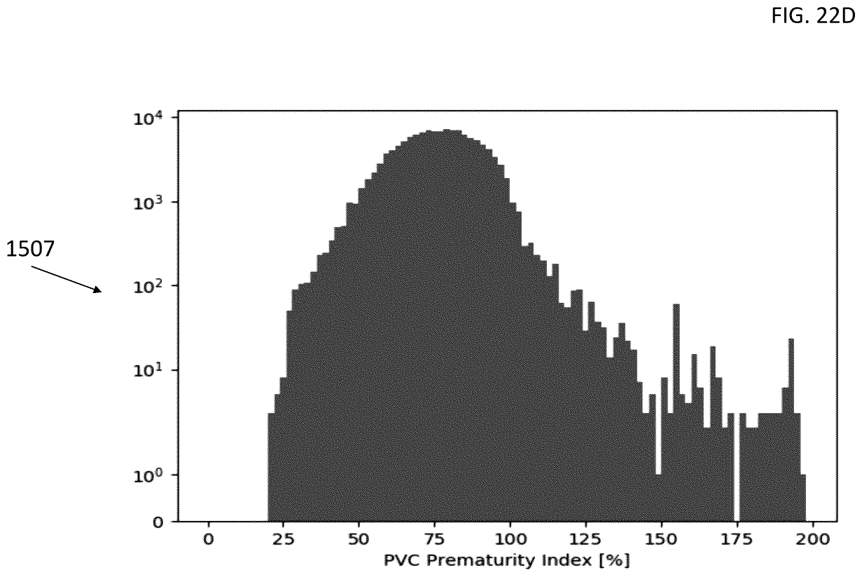

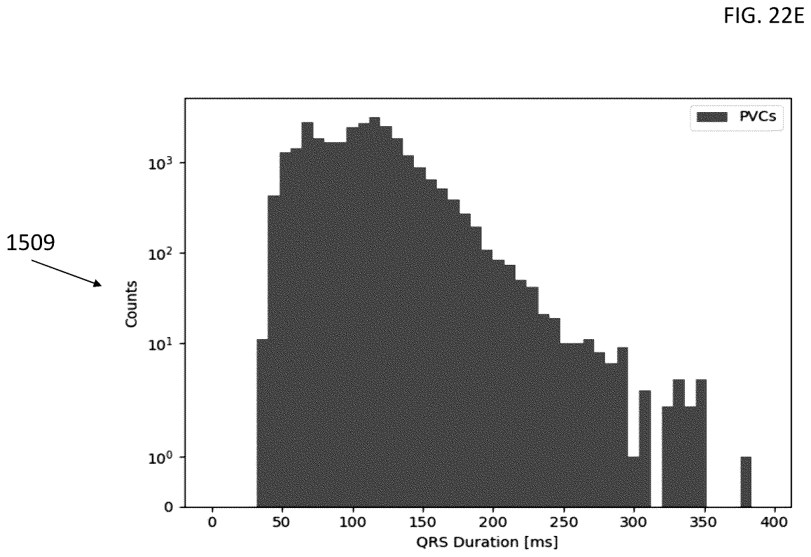

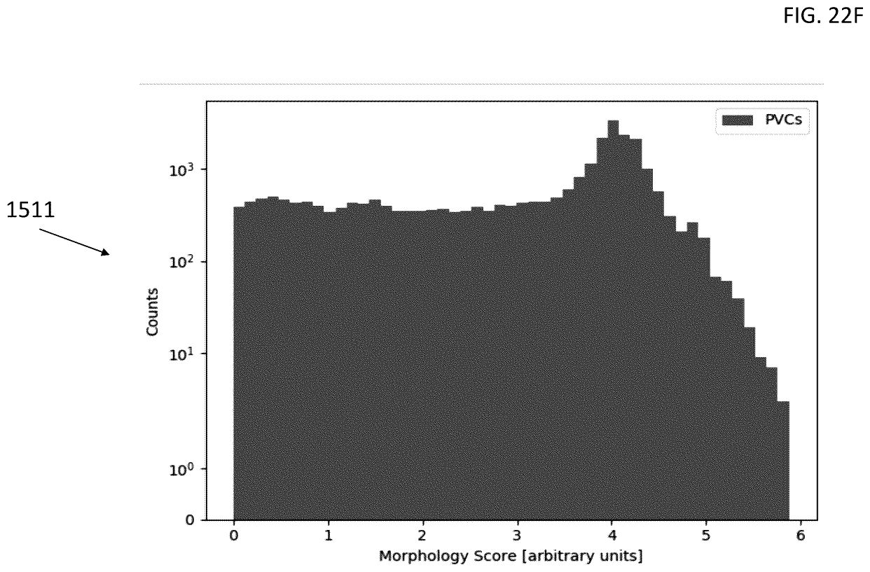

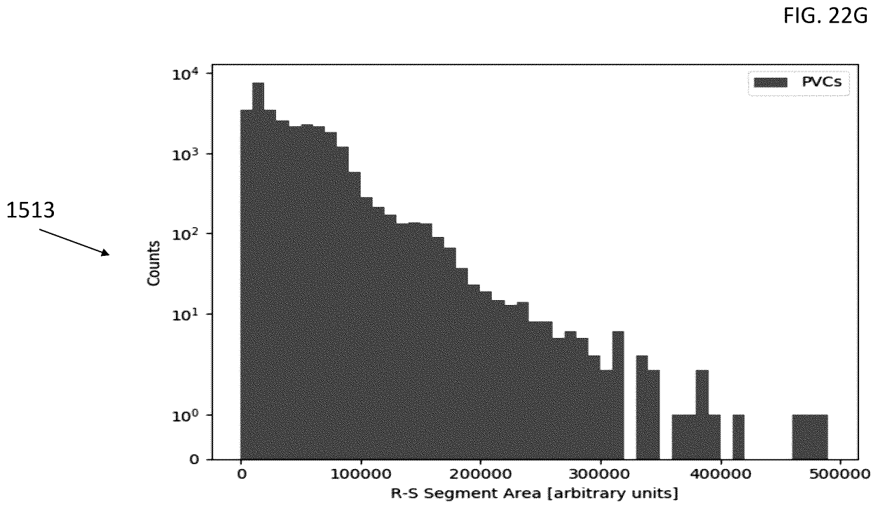

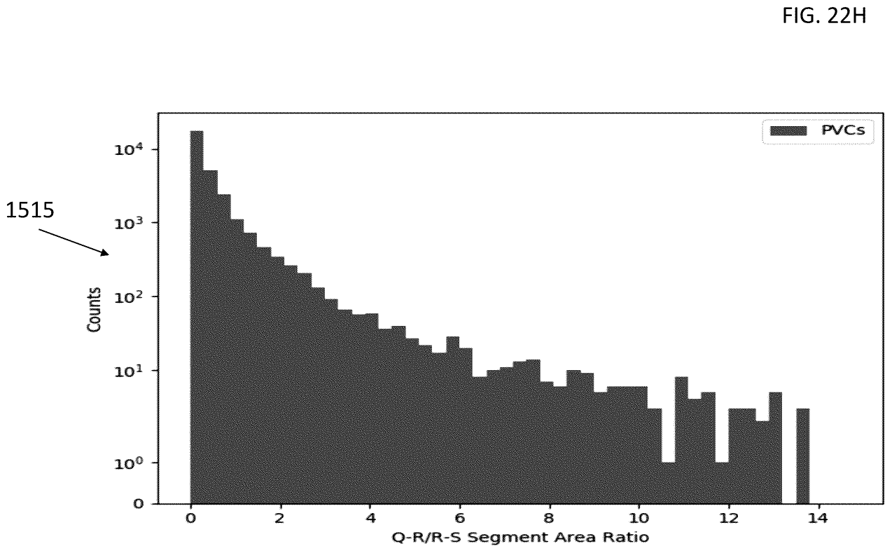

FIG. 22A illustrates patient PVC characteristics data. As shown, morphology scores and their corresponding counts were determined for PVCs of a patient. The illustrated data 1500 provides PVC morphology score distribution for PVC beats in one hour preceding a cardiac event requiring a shock. In some embodiments the PVC morphology score may be determined by applying a convex hull algorithm or the like to characteristics of the PVC.

FIGS. 22B-22G also illustrate patient PVC characteristics data, and two variable correlations for PVC characteristics data. In particular, FIG. 22B illustrates a histogram of R-R duration across PVCs and shows count data 1501 for various R-R durations. A similar histogram to that illustrated in FIG. 22B is expected for R-R duration for previously identified PVCs. FIG. 22C illustrates correlations between current beat R-R duration 1505 and previous beat R-R duration 1503.

FIG. 22D illustrates patient PVC characteristics data and more particularly, a local prematurity index 1507. A local prematurity index can be indicative of how early a PVC beat is. In addition to local prematurity index, PVC characteristics data may include beat characteristics such as a count of the number of normal beats, a count of the number of PVCs, and a determination of the fraction of PVC beats.

As shown in FIG. 22E, patient PVC characteristics data may also include QRS duration counts as shown in the histogram 1509. Patient PVC characteristics data may also include morphology score data as shown in the histogram 1511 illustrated in FIG. 22F.

As illustrated in FIG. 22G, patient PVC characteristic data may also include an area corresponding to a R-S segment for the identified PVCs 1513. And as shown in FIG. 22H, patient PVC characteristic data may also include a histogram 1515 of Q-R to R-S segment area ratio for identified PVCs.

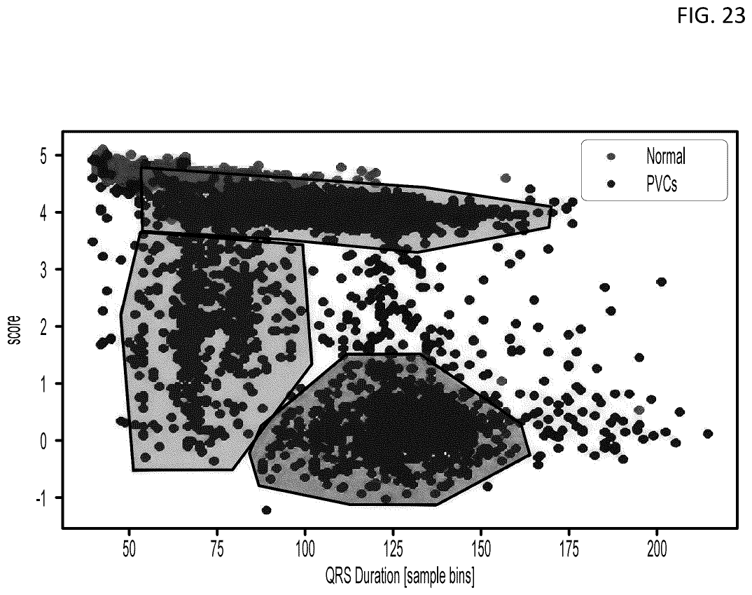

As discussed above, in an example embodiment, clustering algorithms may be applied to PVC characteristics data to determine boundaries and clusters within the PVC characteristics data. Although clusters may be determined from multi-dimensional data (e.g. 7 dimensions), FIG. 23 provides a visual representation of clusters determined based on two dimensions of PVC characteristics data. As illustrated in FIG. 23, the applied clustering algorithm identified three distinct PVC morphologies. For example, a hierarchical density-based spatial clustering of applications with noise (HDBSCAN) clustering algorithm applied to the QRS data produced non-circular clusters that would be approximately represented as shown in FIG. 23. Alternative clustering algorithms may be used.

The unsupervised clustering algorithm may be applied to patient PVC characteristics data for each identified PVC in the identified ECG data points that correspond to one or more PVCs of the patient. Patient PVC characteristics data may include the R-R duration for the PVC, a R-R duration for a previously identified PVC, a local prematurity index, a beat type, QRS duration of the identified PVC, a morphology score for the identified PVC, an area corresponding to a R-S segment for the identified PVC, a ratio between a Q-R segment for the identified PVC to a R-S segment for the identified PVC, and the like.

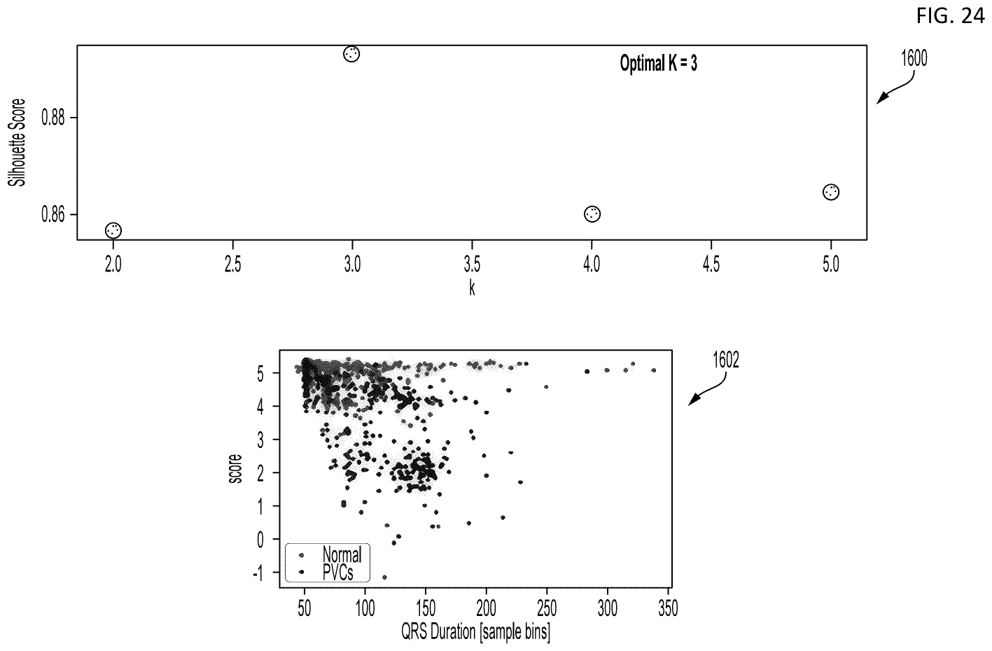

FIG. 24 illustrates the output from a k-means clustering algorithm. As shown in panel 1600, a silhouette score can be determined for each prospective number of groupings (e.g., 2, 3, 4). The silhouette score may provide an indication as to whether the given number of clusters, k, provides adequate separation of data points. A higher silhouette score may indicate a better quality of clustering. As illustrated in panel 1600, a higher peak at k=3 may indicate that the patient has PVCs originating from three focal points. The second panel 1602 in FIG. 24 illustrates a plot of QRS duration and morphology score. As shown the PVCs cluster in three effective groupings, indicating that the patient has PVCs originating from three focal points.

As an alternative or in addition to a silhouette score, in some implementations a gap score may be used as a metric to determine whether the unsupervised learning algorithm has appropriately determined clusters based on the PVC data in order to determine how many focal points are associated with the provided PVC data. A gap score may be determined for each cluster based on the determined distance between points in the cluster. A gap statistic may be calculated across all clusters and maximized. Maximizing the gap statistic may provide the optimal value of k, or the number of clusters, indicating the number of focal points observed in the PVC data.

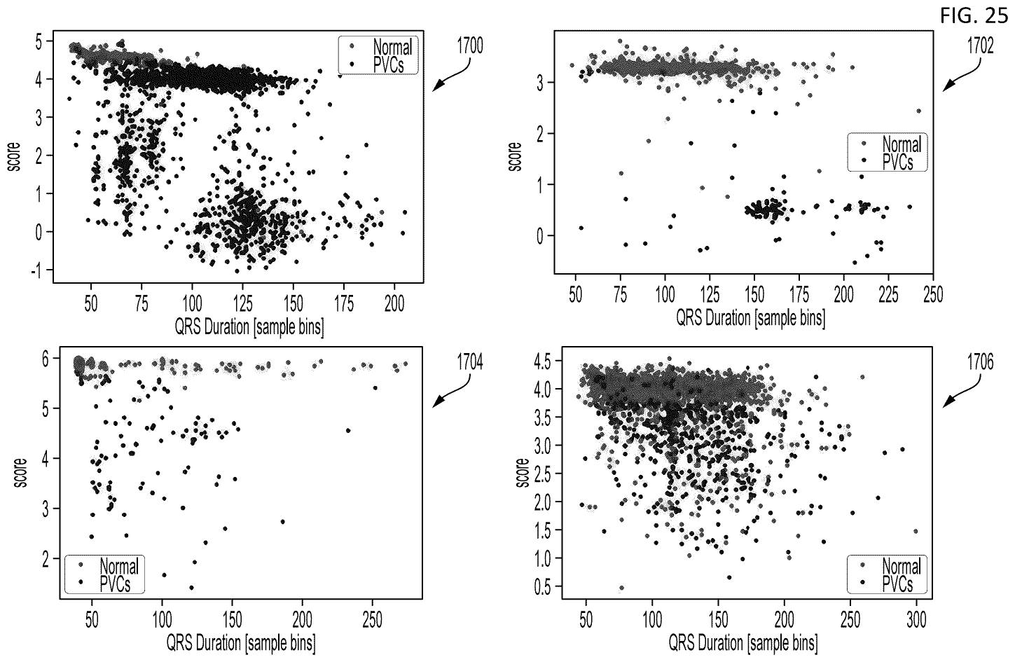

FIG. 25 provides an illustration of PVC data across different patient data sets. For example, although all of the data sets shown in FIG. 25 are for bifocal PVCs, visualizing the underlying QRS duration data alone is insufficient to indicate that the PVCs are of two different morphologies. For example, the QRS duration data in 1700 seems to indicate the presence of more than two focal points. The QRS duration data in 1702 also seems to indicate the presence of more than two focal points. The QRS duration data in 1704 indicates one focal point. And the QRS data in 1706 indicates one focal point.

In some implementations, the processor may be further configured to identify QRS notch data points which correspond to the QRS notches of patient based on the digitized ECG signal. As discussed above, the presence of QRS notches in an ECG signal may indicate a greater risk for having a sudden cardiac arrest event.

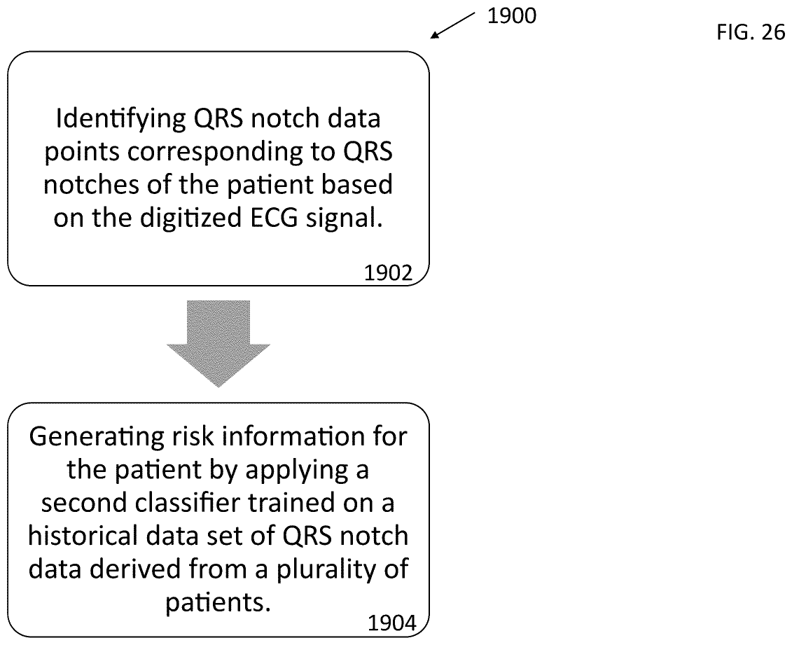

FIG. 26 provides a sample process flow in a cardiac event risk assessment system that analyzes data related to QRS notches to generate and output risk information concerning a future cardiac event such as a sudden cardiac arrest event occurring in a patient.

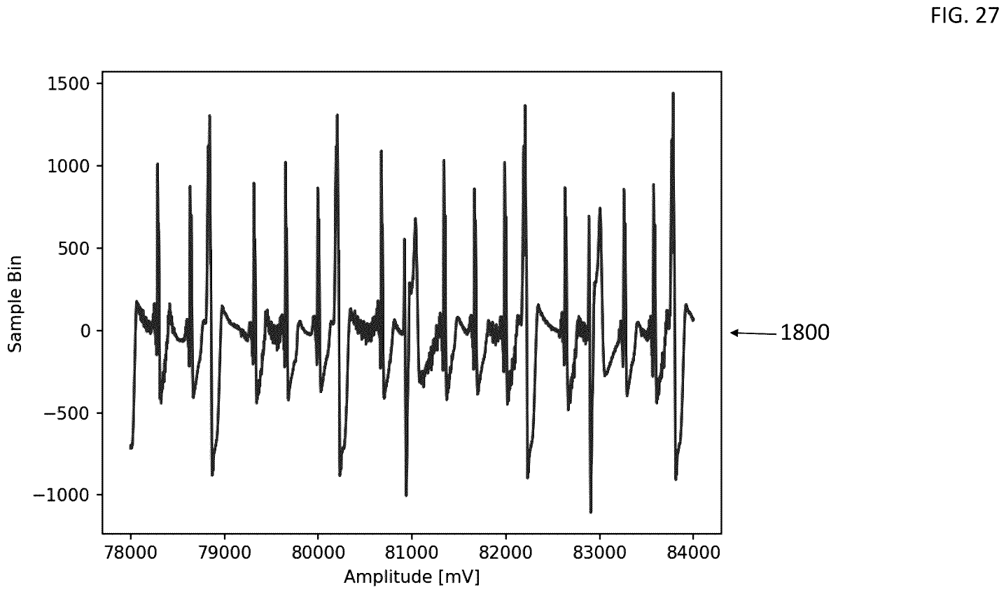

The sample process 1900 shown in FIG. 26 can be implemented by a processor in communication with the wearable cardiac sensing device 102. In implementations, the processor may be part of the wearable cardiac sensing device 102. For example, where the wearable cardiac sensing device 102 includes a cardiac sensing unit 106 and a portable gateway 110, the processor may be implemented by the microcontroller 310 of the cardiac sensing unit 106. As another example, the processor may be implemented in the portable gateway 110. As another example, where the wearable cardiac sensing device 102 includes a garment-based sensing device 118, the processor may be implemented by the processor 518. As another example, the processor may be implemented in the remote server 104. As another example, the processor may be implemented partially by a processor at the wearable cardiac sensing device 102 and partially by a processor at the remote server 104. In some implementations, the sample process illustrated in FIG. 27 may be carried out by the same processor as that used to carry out the sample process illustrated in FIG. 20.

In a first step 1902 of process 1900 illustrated in FIG. 26, the processor may identify QRS notch data points corresponding to QRS notches of the patient based on the digitized ECG signal. For example, the processor may be provided with digitized ECG signal data analogous to that illustrated in FIG. 27.

In some implementations, the processor may identify QRS notch data points by applying wavelet transforms or similar processes. For example, data points corresponding to truncated QRS complexes may be extracted from the digitized ECG signal. A discrete wavelet transform can be applied to the extracted data points in order to transform the truncated QRS complex. Transition points within the transformed QRS complex can be identified. The identified transition points can then be provided to a trained support vector machine classifier. When applied to the identified transition points, the trained support vector machine classifier can identify data points associated with a QRS notch.

The support vector machine classifier can be trained to identify QRS complexes with notches. For example, in some implementations, the support vector machine classifier can be trained on a set of features that includes one or more of normal beats, high frequency direction, high frequency local minima, high frequency amplitudes, low frequency direction, low frequency local minima, and the like.

Turning back to FIG. 26, in a second step 1904, the processor is configured to generate risk information for the patient by applying a second classifier that is trained on a historical data set of QRS notch data derived from a plurality of patients. The second classifier can be configured to generate risk information. The second classifier may include one or more of a logistic regression model, support vector machine model, neural network model, a learning survival model, or the like. The historical data set of QRS notch data may include data derived from a plurality of patients, including a first set of patients that underwent a cardiac event, and a second set of patients that did not undergo a cardiac event.

FIG. 27 provides an illustration of a digitized ECG signal having QRS notches.

In such an embodiment, the classifier generating risk information based on PVC focal points may be modified to include training including the historical data set of QRS notch data. In other embodiments, a second classifier can be trained on the historical data set of QRS notch data to determine additional risk information for the patient, in addition to and separate from the classifier configured to determine risk information for the patient based on PVC focal points.

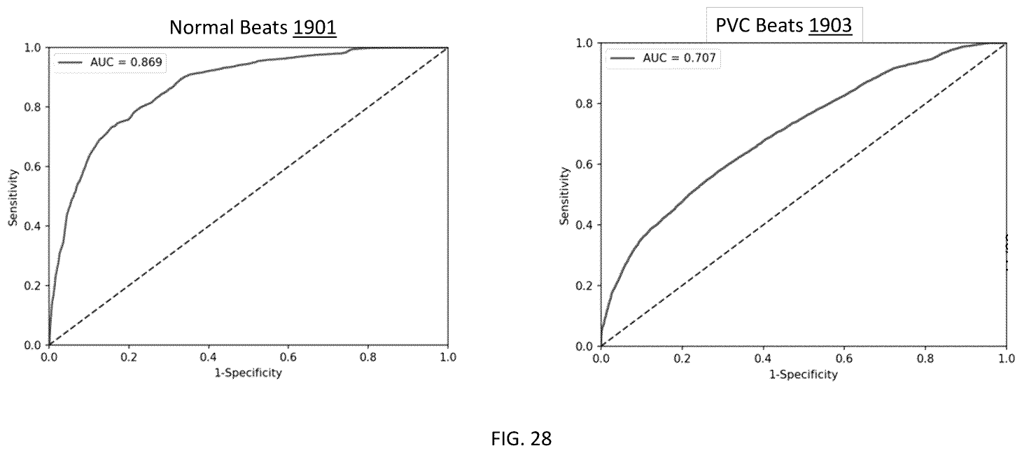

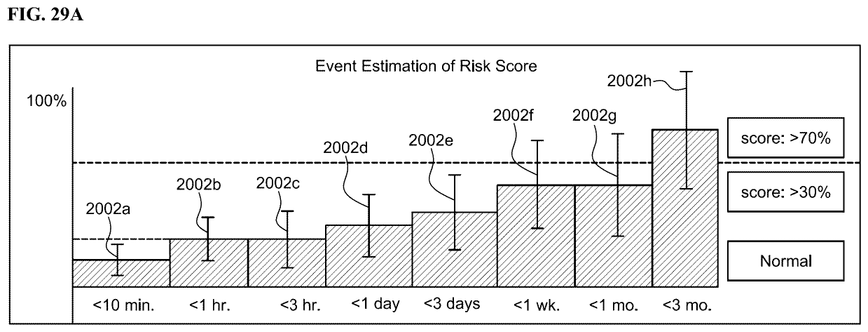

FIG. 28 provides an illustration of PVC notch detection using convex hull features performed on an example dataset including patients wearing a cardiac monitor device. ECG data received from the cardiac monitor device were annotated for notched QRS complexes in normal beats and in PVC beats. A gradient boosting classifier that included a set of frequency-based ECG features along with the convex hull ECG features was applied to the resulting ECG data. As illustrated in FIG. 28, in an example embodiment, the classifier performed with a ROC-AUC of 0.87 for normal beats 1901 and a ROC-AUC of 0.71 for PVC beats 1903. In particular, FIG. 28 illustrates the ROC curves for the classifiers and models trained on normal beats and PVC beats.

As discussed herein, embodiments of the wearable cardiac event risk assessment system may be used to determine the risk of a future adverse cardiac event such as a sudden cardiac arrest event or a risk of cardiac ischemia. As discussed, the risk associated with a future adverse cardiac event may be visually presented to interested parties such as a physician, caregiver, and/or the patient themselves. Risk estimates may be provided in any suitable format. For example, risk estimates may be valid for multiple future time periods, e.g., the next hour, the next three hours, the next 6 hours, the next 24 hours, the next three days, the next week, the next 2 weeks, the next month, the next 3 months, the next 6 months, and/or other configurable duration. In some implementations, the associated risk estimates for the various future time periods can include information reflecting a confidence or other reliability measure for the associated risk estimate, e.g., in the form of a confidence rating or other such metric.

For example, as shown in FIG. 6A, the information may be displayed graphically to allow the subject and/or a caregiver to assess both the current risk and future risk of an adverse cardiac event. FIG. 6A depicts a bar graph can provide a visual representation of example cardiac event estimation of risk scores for multiple time periods. The visual representations of the event estimation of risk scores for the various time periods may include information related to a reliability of the event estimate of risk score, e.g., a confidence value, associated with the corresponding event estimation of risk score. For example, a visual representation 2002a-h of the confidence value may be superimposed on each bar of the bar graph. The visual representations 2002a-h of the confidence values can indicate a likelihood that an event may occur for the associated time period, such that the visual representations that span a relatively greater distance (e.g., the visual representations 2002g and 2002h) indicate a relatively lower confidence in the event estimation of risk score than the visual representations that span a relatively lesser distance (e.g., the visual representations 2002a and 2002b). In some implementations, the ranges of the visual representations 2002a-h of the confidence values may indicate possible error ranges of the corresponding event estimation of risk score. In some examples, confidence values for event estimation of risk scores that correspond to relatively longer time periods (e.g., within one month, within three months) are lower than confidence values for event estimation of risk scores that correspond to relatively shorter time periods (e.g., within ten minutes, within one hour). This may be due to the difficulty involved in making predictions relatively far into the future, for example.

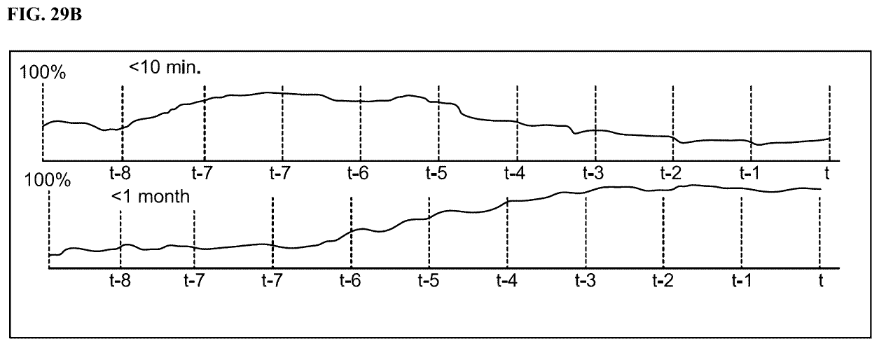

Further, in some examples, historical information about the progression or change in event estimation of risk scores may be provided, examples of which are shown in FIG. 29B. FIG. 29B includes multiple trend lines representing previously calculated event estimation of risk scores over a period of time. For example, in some implementations, the described system can establish an initial baseline risk estimate (and associated confidence rating) for a patient. For example, in implementations, the cardiac monitor can estimate initial and/or baseline values for physiological parameters (e.g., baseline ECG, cardiovibration, and/or lung fluid level recordings) at an initial period of time (e.g., first hour, first two hours, first three hours, first day, or other predetermined period of time) during the beginning of the patient's use of the device. These baseline values can be used to establish a baseline risk estimate for future cardiac events for the patient. Thereafter, the risk estimates can vary during the patient's use of the device. In such cases, the implementations herein can present such changes to the risk estimates with information showing the changes relative to the baseline values. For example, as shown in FIG. 29A and FIG. 29B, event estimation of risk score can be presented in relation to the normal value which is based on the initial baseline risk estimate for the patient.

Embodiments of the present disclosure discuss a cardiac event risk assessment system for assessing future cardiac risk for a patient. As described herein, the system can include a wearable cardiac sensing device configured to be removably, bodily-attached to the patient, the wearable cardiac sensing device configured to sense cardiovibration signals from a patient and provide digitized cardiovibration signals; and a processor in communication with the wearable cardiac sensing device, the processor configured for: identifying cardiovibration data points corresponding to a predetermined physiological marker of the patient based on the digitized cardiovibration signal; analyzing a frequency spectrum corresponding to the identified cardiovibration data points to determine one or more cardiovibration frequency metrics for the patient associated with the predetermined physiological marker of the patient; providing the one or more cardiovibration frequency metrics for the patient to a trained cardiovibration classifier, wherein the trained cardiovibration classifier is trained at least in part on historical cardiovibration frequency metrics associated with the predetermined physiological marker derived from a plurality of patients; and optimizing the trained cardiovibration classifier based on one or more predetermined classifier evaluation metrics; outputting, based on the trained and optimized cardiovibration classifier, risk information concerning a future cardiac ischemia and/or a sudden cardiac arrest event occurring within a predetermined future period of time.

Optionally, the predetermined physiological marker can include a first heart sound (S1) corresponding to closure of the atrioventricular valve of the patient, and/or the predetermined physiological marker can include a second heart sound (S2) corresponding to aortic closure and pulmonic closure for the patient. Optionally, the processor configured for identifying cardiovibration data points can obtain the digitized cardiovibration signal; identify heart beats within the obtained digitized cardiovibration signal by identifying local R-peaks within the obtained digitized cardiovibration signal; and identify a set of points adjacent to the identified local R-peak corresponding to a S1 region for each identified heartbeat. Optionally, identifying cardiovibration data points can include: obtaining the digitized cardiovibration signal; identifying heart beats within the obtained digitized cardiovibration signal by identifying local R-peaks within the obtained digitized cardiovibration signal; identifying a corresponding T-wave for each identified local R-peak; and identifying a set of points adjacent to the identified T-wave corresponding to a S2 region for each identified heartbeat.

Optionally, the processor can be further configured for analyzing the frequency spectrum corresponding to the identified cardiovibration data points to determine one or more cardiovibration frequency metrics for the patient associated with the predetermined physiological marker by performing a fast fourier transform on the identified cardiovibration data points to determine a frequency spectrum for the identified cardiovibration data points, wherein the frequency spectrum decomposes the identified cardiovibration data points into components of different frequencies, and determining a power spectral density for the determined frequency spectrum. Cardiovibration frequency metrics can include a peak frequency, wherein the peak frequency comprises the frequency at which the power spectral density for the identified cardiovibration data points is the highest. Cardiovibration frequency metrics can include a width, wherein the width includes the difference between the highest and lowest frequencies present in the identified cardiovibration data points. Cardiovibration frequency metrics can include the mean frequency, wherein the mean frequency comprises the average frequency of the identified cardiovibration data points. Cardiovibration frequency metrics can include one or more of a standard deviation, or an entropy measure, or a spectrum bandwidth measure of the frequency spectrum for the identified cardiovibration data points. A cardiovibration classifier can include a thresholding algorithm. The cardiovibration classifier can include one or a combination of a logistic regression model, support vector machine model, neural network model, or a learning survival model. In some embodiments, historical cardiovibration frequency metrics associated with the predetermined physiological marker derived from a plurality of patients can include a first set of cardiovibration frequency metrics associated with a group of patients who experienced a cardiac arrest and a second set of cardiovibration frequency metrics associated with a group of patients who did not experience a cardiac arrest. In some embodiments, optimizing the trained cardiovibration classifier can include adjusting one or more thresholds, weights or metrics of the trained cardiovibration classifier to improve the one or more predetermined classifier metrics, where the one or more predetermined classifier metrics includes an indicator for the performance of the cardiovibration classifier. The one or more predetermined classifier metrics can include at least one of a probability evaluation metric, a concordance index, a sensitivity metric, or an area under the curve metric.

The risk information can include a binary classification of high risk or low risk, a plurality of classes including high-risk, medium-risk, or low-risk, a risk score including a percentage between 0 and 100, and/or a risk score including a probabilistic measure between 0 and 1. The risk information can include a survival function including a probability that a patient will remain free of having a cardiac ischemic and/or sudden cardiac arrest event after the predetermined future period of time. The risk information can include a hazard function indicative of a frequency or rate that a cardiac ischemia and/or sudden cardiac arrest event will occur after the predetermined future period of time has elapsed without the patient having a cardiac ischemia and/or sudden cardiac arrest event. The predetermined future period of time can include one of one month, 14-days, 10-days, 5-days, 3-days, 1-day, 20 hours, 16 hours, 10 hours, 5 hours, 3 hours, or 1 hour. The predetermined future period of time can be based on a time span derived from training data comprising the historical cardiovibration frequency metrics and known cardiac ischemia and/or a sudden cardiac arrest events. Risk information can include at least one of displaying the risk information on a display screen, generating a medical report comprising the risk information, initiating an alarm responsive to the risk information, or initiating a therapeutic protocol responsive to the risk information.

The wearable cardiac sensing device can include a garment configured to be worn around the patient's torso for an extended period of time. The wearable cardiac sensing device can include one or more cardiovibration sensors configured to sense cardiovibration signals. The cardiovibration sensors can be removably mounted onto the garment, or at least a portion of the one or more cardiovibration sensors are permanently integrated into the garment. Optionally, the wearable cardiac sensing device can include a removable adhesive patch configured to be adhere to skin of the patient. At least a portion of the one or more cardiovibration sensors can be configured to be removably mounted onto the removable adhesive patch. The wearable cardiac sensing device can include a cardiac sensing unit incorporating the portion of the one or more cardiovibration sensors, the cardiac sensing unit configured to be removably mounted onto the removable adhesive patch. The wearable cardiac sensing device can include the processor. The wearable cardiac sensing device can be in communication with a remote server and the processor can be further configured to transmit the cardiovibration data points to the remote server. The system can include a remote server in communication with the wearable cardiac sensing device, which can include the processor. In some embodiments, the system can include a portable gateway configured to facilitate communication between the remote server and the wearable cardiac sensing device. The wearable cardiac sensing device can include ECG electrodes and be configured to sense electrocardiogram (ECG) signals from the patient via the ECG electrodes. The processor can be configured to analyze the ECG signals to determine one or more ECG features, and provide the one or more ECG features to the trained cardiovibration classifier, where the trained cardiovibration classifier is further trained on historical ECG features derived from the plurality of patients. The one or more ECG features can include at least one of one or more QRS metrics, or one or more T wave metrics.

In some embodiments, a cardiac event risk assessment system includes a wearable cardiac sensing device configured to be removably, bodily-attached to the patient, the wearable cardiac sensing device configured to sense electrocardiogram (ECG) signals from a patient and provide digitized ECG signals; and a processor in communication with the wearable cardiac sensing device. The processor can be configured to identify ECG data points corresponding to premature ventricular contractions (PVCs) of the patient based on the digitized ECG signal; determine one or more PVC patterns based on the identified ECG data points corresponding to the PVCs of the patient; determine one or more of a count or a burden associated with the one or more PVC patterns; and output risk information concerning sudden cardiac arrest event occurring within a predetermined future period of time by applying a trained machine learning algorithm to the determined one or more of the count or the burden associated with the patient, wherein the trained machine learning algorithm is trained at least in part on historical PVC pattern data derived from a plurality of patients.

Optionally, the PVC pattern includes a PVC run, wherein a PVC run is indicative of the number of consecutive PVCs. Optionally, the PVC run is one of a singlet, couplet, triplet, quadruplet, or Non-sustained ventricular tachycardia (NSVT). The PVC pattern can include a n-geminy, wherein an n-geminy is indicative of the interval between PVCs. N-geminy is one of a bigeminy, trigeminy, quadrageminy, or 5-geminy. Optionally, the burden associated with the one or more PVC patterns can include a count associated with the one or more PVC patterns over the total time of the digitized ECG signal. In some embodiments historical PVC pattern data derived from a plurality of patients includes a first set of PVC pattern data associated with a group of patients who experienced a sudden cardiac arrest event and a second set of PVC pattern data associated with a group of patients who did not experience a sudden cardiac arrest event.

The risk information can include a binary classification of high risk or low risk, a plurality of classes including high-risk, medium-risk, or low-risk, a risk score including a percentage between 0 and 100, and/or a risk score including a probabilistic measure between 0 and 1. The risk information can include a survival function including a probability that a patient will remain free of having a cardiac ischemic and/or sudden cardiac arrest event after the predetermined future period of time. The risk information can include a hazard function indicative of a frequency or rate that a cardiac ischemia and/or sudden cardiac arrest event will occur after the predetermined future period of time has elapsed without the patient having a cardiac ischemia and/or sudden cardiac arrest event. The predetermined future period of time can include one of one month, 14-days, 10-days, 5-days, 3-days, 1-day, 20 hours, 16 hours, 10 hours, 5 hours, 3 hours, or 1 hour. Risk information can include at least one of displaying the risk information on a display screen, generating a medical report comprising the risk information, initiating an alarm responsive to the risk information, or initiating a therapeutic protocol responsive to the risk information. The predetermined future period of time is based on a time span derived from training data comprising the PVC pattern data and known sudden cardiac arrest events.

The wearable cardiac sensing device can include a garment configured to be worn around the patient's torso for an extended period of time. The wearable cardiac sensing device can include one or more sensors configured to sense ECG signals. The sensors can be removably mounted onto the garment, or at least a portion of the one or more sensors are permanently integrated into the garment. Optionally, the wearable cardiac sensing device can include a removable adhesive patch configured to be adhere to skin of the patient. At least a portion of the one or more sensors can be configured to be removably mounted onto the removable adhesive patch. The wearable cardiac sensing device can include a cardiac sensing unit incorporating the portion of the one or more sensors, the cardiac sensing unit configured to be removably mounted onto the removable adhesive patch. The wearable cardiac sensing device can include the processor. The wearable cardiac sensing device can be in communication with a remote server and the processor can be further configured to transmit the ECG data to the remote server. The system can include a remote server in communication with the wearable cardiac sensing device, which can include the processor. In some embodiments, the system can include a portable gateway configured to facilitate communication between the remote server and the wearable cardiac sensing device. The processor can be configured to analyze the ECG signals to determine one or more ECG features.

In some embodiments, a cardiac event risk assessment system for assessing future cardiac risk for a patient can include a wearable cardiac sensing device configured to be removably, bodily-attached to the patient, the wearable cardiac sensing device configured to sense electrocardiogram (ECG) signals from a patient and provide digitized ECG signals and a processor in communication with the wearable cardiac sensing device. The processor can be configured to: identify ECG data points corresponding to premature ventricular contractions (PVCs) of the patient based on the digitized ECG signal; determine multi-focal PVC data from the identified ECG data points corresponding to PVCs by applying an unsupervised machine learning algorithm, wherein the unsupervised machine learning algorithm is trained to determine multiple focal points within a set of PVCs; and generate risk information for the patient indicative of a future sudden cardiac arrest event by applying a trained classifier to the determined multi-focal PVC data, wherein the classifier is trained on a historical data set of multi-focal PVC data derived from a plurality of patients. The processor can be further configured to identify QRS notch data points corresponding to QRS notches of the patient based on the digitized ECG signal, where generating risk information for the patient includes applying a second classifier trained on a historical data set of QRS notch data derived from a plurality of patients.

Optionally, the unsupervised machine learning algorithm includes an unsupervised clustering algorithm. Optionally, determining multi-focal PVC data can include generating patient PVC characteristics data for each identified PVC in the identified ECG data points corresponding to one or more PVCs of the patient; and applying the trained K-means unsupervised clustering algorithm on the generated patient PVC characteristics data.

In some embodiments patient PVC characteristics data includes one or more of a R-R duration for the identified PVC, a R-R duration for a previously identified PVC, a local prematurity index, a beat type, QRS duration for the identified PVC, a morphology score for the identified PVC, an area corresponding to a R-S segment for the identified PVC, or a ratio between a Q-R segment for the identified PVC to a R-S segment for the identified PVC; and the unsupervised clustering algorithm includes a k-means clustering algorithm. Additionally or alternatively, the patient PVC characteristics data includes one or more of a morphology score for the identified PVC, and a QRS width for the identified PVC, and wherein the unsupervised clustering algorithm includes a 2-means unsupervised clustering algorithm.

The processor can be further configured to generate a proposed clustering of PVCs based on the application of the unsupervised clustering algorithm to the patient PVC characteristics data; and generate a final clustering of PVCs based on at least one of maximizing a silhouette coefficient for the proposed clustering of PVCs and maximizing a gap score for PVCs in each of the proposed clusters. Optionally, the determined multi-focal PVC data includes a number of focal points corresponding to the identified ECG data points corresponding to PVCs, or an indicator of the presence or absence of multiple focal points within a set of PVCs.

The historical data set of multi-focal PVC data derived from a plurality of patients includes a first set of multi-focal PVC data associated with a group of patients who experienced a sudden cardiac arrest event and a second set of multi-focal PVC data associated with a group of patients who did not experience a sudden cardiac arrest event.

The risk information can include a binary classification of high risk or low risk, a plurality of classes including high-risk, medium-risk, or low-risk, a risk score including a percentage between 0 and 100, and/or a risk score including a probabilistic measure between 0 and 1. The risk information can include a survival function including a probability that a patient will remain free of having a cardiac ischemic and/or sudden cardiac arrest event after the predetermined future period of time. The risk information can include a hazard function indicative of a frequency or rate that a cardiac ischemia and/or sudden cardiac arrest event will occur after the predetermined future period of time has elapsed without the patient having a cardiac ischemia and/or sudden cardiac arrest event. The predetermined future period of time can include one of one month, 14-days, 10-days, 5-days, 3-days, 1-day, 20 hours, 16 hours, 10 hours, 5 hours, 3 hours, or 1 hour. Risk information can include at least one of displaying the risk information on a display screen, generating a medical report comprising the risk information, initiating an alarm responsive to the risk information, or initiating a therapeutic protocol responsive to the risk information. The predetermined future period of time can be based on a time span derived from training data comprising the historical data set of multi-focal PVC data and known sudden cardiac arrest events.

In some embodiments identifying QRS notch data points corresponding to QRS notches of the patient based on the digitized ECG signal further and can include: extracting data points from the digitized ECG signal corresponding to truncated QRS complexes; transforming the truncated QRS complex by applying a discrete wavelet transform to the extracted data points; identifying transition points in the transformed QRS complex; and identifying data points associated with a QRS notch by applying a trained support vector machine classifier to the identified transition points. A support vector machine can be trained to identify QRS complexes with notches. The support vector machine can be trained on a set of features, wherein the set of features includes normal beats, high frequency direction, high frequency local minima, high frequency amplitudes, low frequency direction and low frequency local minima.

The historical data set of QRS notch data can be derived from a plurality of patients includes a first set of QRS notch data associated with a group of patients who experienced a sudden cardiac arrest event and a second set of QRS notch data associated with a group of patients who did not experience a sudden cardiac arrest event.

The wearable cardiac sensing device can include a garment configured to be worn around the patient's torso for an extended period of time. The wearable cardiac sensing device can include one or more sensors configured to sense ECG signals. The sensors can be removably mounted onto the garment, or at least a portion of the one or more sensors are permanently integrated into the garment. Optionally, the wearable cardiac sensing device can include a removable adhesive patch configured to be adhere to skin of the patient. At least a portion of the one or more sensors can be configured to be removably mounted onto the removable adhesive patch. The wearable cardiac sensing device can include a cardiac sensing unit incorporating the portion of the one or more sensors, the cardiac sensing unit configured to be removably mounted onto the removable adhesive patch. The wearable cardiac sensing device can include the processor. The wearable cardiac sensing device can be in communication with a remote server and the processor can be further configured to transmit the ECG data to the remote server. The system can include a remote server in communication with the wearable cardiac sensing device, which can include the processor. In some embodiments, the system can include a portable gateway configured to facilitate communication between the remote server and the wearable cardiac sensing device. The processor can be configured to analyze the ECG signals to determine one or more ECG features.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

We claim:

1. A cardiac event risk assessment system for assessing future cardiac risk for a patient, the system comprising:
a wearable cardiac sensing device configured to be removably, bodily-attached to the patient, the wearable cardiac sensing device configured to sense cardiovibration signals from a patient and provide digitized cardiovibration signals; and
a processor in communication with the wearable cardiac sensing device, the processor configured for:
identifying cardiovibration data points corresponding to a predetermined physiological marker of the patient based on the digitized cardiovibration signal;
analyzing a frequency spectrum corresponding to the identified cardiovibration data points to determine one or more cardiovibration frequency metrics for the patient associated with the predetermined physiological marker of the patient;
providing the one or more cardiovibration frequency metrics for the patient to a trained cardiovibration classifier, wherein the trained cardiovibration classifier is trained at least in part on historical cardiovibration frequency metrics associated with the predetermined physiological marker derived from a plurality of patients;
optimizing the trained cardiovibration classifier based on one or more predetermined classifier evaluation metrics; and
outputting, based on the trained and optimized cardiovibration classifier, risk information concerning a future cardiac ischemia and/or a sudden cardiac arrest event occurring within a predetermined future period of time.

2. The system of claim 1, wherein the predetermined physiological marker comprises a first heart sound (S1) corresponding to closure of the atrioventricular valve of the patient, and wherein the processor configured for identifying cardiovibration data points is further configured for:
obtaining the digitized cardiovibration signal;
identifying heart beats within the obtained digitized cardiovibration signal by identifying local R-peaks within the obtained digitized cardiovibration signal; and
identifying a set of points adjacent to the identified local R-peak corresponding to a S1 region for each identified heartbeat.

3. The system of claim 1, wherein the predetermined physiological marker comprises a second heart sound (S2) corresponding to aortic closure and pulmonic closure for the patient, and wherein the processor configured for identifying cardiovibration data points is further configured for:
obtaining the digitized cardiovibration signal;
identifying heart beats within the obtained digitized cardiovibration signal by identifying local R-peaks within the obtained digitized cardiovibration signal;
identifying a corresponding T-wave for each identified local R-peak; and
identifying a set of points adjacent to the identified T-wave corresponding to a S2 region for each identified heartbeat.

4. The system of claim 1, wherein the processor is further configured for analyzing the frequency spectrum corresponding to the identified cardiovibration data points to determine one or more cardiovibration frequency metrics for the patient associated with the predetermined physiological marker comprises:
performing a fast fourier transform on the identified cardiovibration data points to determine a frequency spectrum for the identified cardiovibration data points, wherein the frequency spectrum decomposes the identified cardiovibration data points into components of different frequencies; and
determining a power spectral density for the determined frequency spectrum.

5. The system of claim 4, wherein the cardiovibration frequency metrics comprises at least one of a peak frequency, a width, a mean frequency, a standard deviation, an entropy measure, or a spectrum bandwidth measure of the frequency spectrum for the identified cardiovibration data points, wherein the peak frequency comprises the frequency at which the power spectral density for the identified cardiovibration data points is the highest, wherein the width comprises the difference between the highest and lowest frequencies present in the identified cardiovibration data points, and wherein the mean frequency comprises the average frequency of the identified cardiovibration data points.

6. The system of claim 1, wherein the cardiovibration classifier comprises one or a combination of a thresholding algorithm, a logistic regression model, support vector machine model, neural network model, or a learning survival model.

7. The system of claim 1, wherein the historical cardiovibration frequency metrics associated with the predetermined physiological marker derived from a plurality of patients comprises a first set of cardiovibration frequency metrics associated with a group of patients who experienced a cardiac arrest and a second set of cardiovibration frequency metrics associated with a group of patients who did not experience a cardiac arrest.

8. The system of claim 1, wherein optimizing the trained cardiovibration classifier comprises adjusting one or more thresholds, weights or metrics of the trained cardiovibration classifier to improve the one or more predetermined classifier metrics, wherein the one or more predetermined classifier metrics comprises an indicator for the performance of the cardiovibration classifier.

9. The system of claim 1, wherein the one or more predetermined classifier metrics comprises at least one of a probability evaluation metric, a concordance index, a sensitivity metric, or an area under the curve metric.

10. The system of claim 1 wherein the risk information comprises one or more of: (i) a binary classification of high risk or low risk, (ii) a plurality of classes comprising a high-risk, medium-risk or low-risk, or a risk score, or (iii) a risk score comprising a percentage between 0 and 100 or a probabilistic measure between 0 and 1.

11. The system of claim 1, wherein the risk information comprises a survival function including a probability that a patient will remain free of having a cardiac ischemic and/or sudden cardiac arrest event after the predetermined future period of time, or a hazard function indicative of a frequency or rate that a cardiac ischemia and/or sudden cardiac arrest event will occur after the predetermined future period of time has elapsed without the patient having a cardiac ischemia and/or sudden cardiac arrest event.

12. The system of claim 1, wherein the predetermined future period of time comprises one of one month, 14-days, 10-days, 5-days, 3-days, 1-day, 20 hours, 16 hours, 10 hours, 5 hours, 3 hours, or 1 hour, and/or a period of time based on a time span derived from training data comprising the historical cardiovibration frequency metrics and known cardiac ischemia and/or a sudden cardiac arrest events.

13. The system of claim 1, wherein outputting the risk information comprises at least one of displaying the risk information on a display screen, generating a medical report comprising the risk information, initiating an alarm responsive to the risk information, or initiating a therapeutic protocol responsive to the risk information.

14. The system of claim 1, the wearable cardiac sensing device further comprises:
a garment configured to be worn around the patient's torso for an extended period of time;
one or more cardiovibration sensors configured to sense cardiovibration signals, wherein at least a portion of the one or more cardiovibration sensors is configured to be removably mounted onto or permanently integrated into the garment;
one or more electrocardiogram (ECG) electrodes configured to sense electrocardiogram signals from the patient via the ECG electrodes.

15. The system of claim 1, wherein the wearable cardiac sensing device further comprises a removable adhesive patch configured to be adhere to skin of the patient, wherein at least a portion of one or more cardiovibration sensors are configured to be removably mounted onto the removable adhesive patch.

16. The system of claim 1, wherein the processor is further configured for
analyzing the ECG signals to determine one or more ECG features comprising at least one or more QRS metrics or one or more T-wave metrics, and provide the one or more ECG features to the trained cardiovibration classifier, wherein the trained cardiovibration classifier is further trained on historical ECG features derived from the plurality of patients.

17. A cardiac event risk assessment system for assessing future cardiac risk for a patient, the system comprising:
a wearable cardiac sensing device configured to be removably, bodily-attached to the patient, the wearable cardiac sensing device configured to sense electrocardiogram (ECG) signals from a patient and provide digitized ECG signals; and
a processor in communication with the wearable cardiac sensing device, the processor configured for:
identifying ECG data points corresponding to premature ventricular contractions (PVCs) of the patient based on the digitized ECG signal;
determining one or more PVC patterns based on the identified ECG data points corresponding to the PVCs of the patient;
determining one or more of a count or a burden associated with the one or more PVC patterns; and
outputting risk information concerning sudden cardiac arrest event occurring within a predetermined future period of time by applying a trained machine learning algorithm to the determined one or more of the count or the burden associated with the patient, wherein the trained machine learning algorithm is trained at least in part on historical PVC pattern data derived from a plurality of patients.

18. The system of claim 17, wherein a PVC pattern comprises a PVC run, wherein a PVC run is indicative of the number of consecutive PVCs, and the PVC run is one of a singlet, couplet, triplet, quadruplet, or Non-sustained ventricular tachycardia (NSVT).

19. The system of claim 17, wherein a PVC pattern comprises a n-geminy, wherein an n-geminy is indicative of the interval between PVCs, and the n-geminy is one of a bigeminy, trigeminy, quadrageminy, or 5-geminy.

20. The system of claim 17, wherein a burden associated with the one or more PVC patterns comprises a count associated with the one or more PVC patterns over the total time of the digitized ECG signal.

21. The system of claim 17, wherein the historical PVC pattern data derived from a plurality of patients comprises a first set of PVC pattern data associated with a group of patients who experienced a sudden cardiac arrest event and a second set of PVC pattern data associated with a group of patients who did not experience a sudden cardiac arrest event.

22. The system of claim 17 wherein the risk information comprises one or more of a binary classification of high risk or low risk, a plurality of classes comprising a high-risk, medium-risk, or low risk, a risk score comprising a percentage between 0 and 100, or a risk score comprising a probabilistic measure between 0 and 1.

23. The system of claim 17, wherein the risk information comprises a survival function including a probability that a patient will remain free of having a sudden cardiac arrest event after the predetermined future period of time, or the risk information comprises a hazard function indicative of a frequency or a rate that a sudden cardiac arrest event will occur after the predetermined future period of time has elapsed without the patient having a sudden cardiac arrest event.

24. The system of claim 17, wherein the risk of the future sudden cardiac arrest event is determined for a predetermined future period of time, comprising one of one month, 14-days, 10-days, 5-days, 3-days, 1-day, 20 hours, 16 hours, 10 hours, 5 hours, 3 hours, or 1 hour, and is based on a time span derived from training data comprising the PVC pattern data and known sudden cardiac arrest events.

25. The system of claim 17, wherein outputting the risk information comprises at least one of displaying the risk information on a display screen, generating a medical report comprising the risk information, initiating an alarm responsive to the risk information, or initiating a therapeutic protocol responsive to the risk information.

26. The system of claim 17, wherein the wearable cardiac sensing device further comprises:
a garment configured to be worn around the patient's torso for an extended period of time; and
one or more sensors configured to sense physiological signals, wherein the physiological signals comprises electrocardiogram (ECG) signals, wherein at least a portion of the one or more sensors are configured to be removably mounted onto or permanently integrated into the garment.

27. The system of claim 26, wherein the wearable cardiac sensing device further comprises a removable adhesive patch configured to be adhere to skin of the patient, and at least a portion of the plurality of sensors are configured to be removably mounted onto the removable adhesive patch.

28. The system of claim 27, wherein the wearable cardiac sensing device further comprises a cardiac sensing unit incorporating the portion of the one or more sensors, the cardiac sensing unit configured to be removably mounted onto the removable adhesive patch.

29. A cardiac event risk assessment system for assessing future cardiac risk for a patient, the system comprising:
a wearable cardiac sensing device configured to be removably, bodily-attached to the patient, the wearable cardiac sensing device configured to sense electrocardiogram (ECG) signals from a patient and provide digitized ECG signals; and
a processor in communication with the wearable cardiac sensing device, the processor configured for:
identifying ECG data points corresponding to premature ventricular contractions (PVCs) of the patient based on the digitized ECG signal;
determining multi-focal PVC data from the identified ECG data points corresponding to PVCs by applying an unsupervised machine learning algorithm, wherein the unsupervised machine learning algorithm is trained to determine multiple focal points within a set of PVCs; and
generating risk information for the patient indicative of a future sudden cardiac arrest event by applying a trained classifier to the determined multi-focal PVC data, wherein the classifier is trained on a historical data set of multi-focal PVC data derived from a plurality of patients.

30. The cardiac event risk assessment system of claim 29, wherein the processor is configured for:
identifying QRS notch data points corresponding to QRS notches of the patient based on the digitized ECG signal;
wherein generating risk information for the patient comprises applying a second classifier trained on a historical data set of QRS notch data derived from a plurality of patients.

31. The system of claim 29, wherein the unsupervised machine learning algorithm comprises an unsupervised clustering algorithm.

32. The system of claim 29, wherein determining multi-focal PVC data further comprises:
generating patient PVC characteristics data for each identified PVC in the identified ECG data points corresponding to one or more PVCs of the patient; and
applying the trained K-means unsupervised clustering algorithm on the generated patient PVC characteristics data.

33. The system of claim 32, wherein the patient PVC characteristics data comprises one or more of a R-R duration for the identified PVC, a R-R duration for a previously identified PVC, a local prematurity index, a beat type, QRS duration for the identified PVC, a morphology score for the identified PVC, an area corresponding to a R-S segment for the identified PVC, or a ratio between a Q-R segment for the identified PVC to a R-S segment for the identified PVC; and wherein the unsupervised clustering algorithm comprises a k-means clustering algorithm.

34. The system of claim 32, wherein the patient PVC characteristics data comprises one or more of a morphology score for the identified PVC, and a QRS width for the identified PVC, and wherein the unsupervised clustering algorithm comprises a 2-means unsupervised clustering algorithm.

35. The system of claim 32, further comprising:
generating a proposed clustering of PVCs based on the application of the unsupervised clustering algorithm to the patient PVC characteristics data; and
generating a final clustering of PVCs based on at least one of maximizing a silhouette coefficient for the proposed clustering of PVCs and maximizing a gap score for PVCs in each of the proposed clusters.

36. The system of claim 32, wherein the determined multi-focal PVC data comprises a number of focal points corresponding to the identified ECG data points corresponding to PVCs, or an indicator of the presence or absence of multiple focal points within a set of PVCs.

37. The system of claim 29, wherein the historical data set of multi-focal PVC data derived from a plurality of patients comprises a first set of multi-focal PVC data associated with a group of patients who experienced a sudden cardiac arrest event and a second set of multi-focal PVC data associated with a group of patients who did not experience a sudden cardiac arrest event.

38. The system of claim 29 wherein the risk information comprises one or more of a binary classification of high risk or low risk, a risk classification including a plurality of classes including a high-risk, medium-risk, or low-risk, a risk score comprising a percentage between 0 and 100, or a risk score comprising a probabilistic measure between 0 and 1.

39. The system of claim 29, wherein the risk information comprises a survival function including a probability that a patient will remain free of having a sudden cardiac arrest event after a predetermined future period of time, or the risk information comprises a hazard function indicative of a frequency or rate that a cardiac ischemia and/or sudden cardiac arrest event will occur after a predetermined future period of time has elapsed without the patient having a sudden cardiac arrest event.

40. The system of claim 29, wherein the risk of the future sudden cardiac arrest event is determined for a predetermined future period of time comprising one of one month, 14-days, 10-days, 5-days, 3-days, 1-day, 20 hours, 16 hours, 10 hours, 5 hours, 3 hours, or 1 hour, wherein the predetermined future period of time is based on a time span derived from training data comprising the historical data set of multi-focal PVC data and known sudden cardiac arrest events.

41. The system of claim 29, wherein outputting the risk information comprises at least one of displaying the risk information on a display screen, generating a medical report comprising the risk information, initiating an alarm responsive to the risk information, or initiating a therapeutic protocol responsive to the risk information.

42. The system of claim 30, wherein identifying QRS notch data points corresponding to QRS notches of the patient based on the digitized ECG signal further comprises:
extracting data points from the digitized ECG signal corresponding to truncated QRS complexes;
transforming the truncated QRS complex by applying a discrete wavelet transform to the extracted data points;
identifying transition points in the transformed QRS complex; and
identifying data points associated with a QRS notch by applying a trained support vector machine classifier to the identified transition points.

43. The system of claim 42, wherein the support vector machine is trained to identify QRS complexes with notches and is trained on a set of features, wherein the set of features comprises normal beats, high frequency direction, high frequency local minima, high frequency amplitudes, low frequency direction and low frequency local minima.

44. The system of claim 42, wherein the historical data set of QRS notch data derived from a plurality of patients comprises a first set of QRS notch data associated with a group of patients who experienced a sudden cardiac arrest event and a second set of QRS notch data associated with a group of patients who did not experience a sudden cardiac arrest event.

45. The system of claim 29, wherein the wearable cardiac sensing device further comprises:
a garment configured to be worn around the patient's torso for an extended period of time; and
one or more sensors configured to sense physiological signals, wherein the physiological signals comprises electrocardiogram (ECG) signals, wherein at least a portion of the one or more sensors are configured to be one of removably mounted onto or permanently integrated into the garment.

46. The system of claim 45, wherein the wearable cardiac sensing device further comprises a removable adhesive patch configured to be adhere to skin of the patient and at least a portion of the plurality of sensors are configured to be removably mounted onto the removable adhesive patch.

47. The system of claim 46, wherein the wearable cardiac sensing device further comprises a cardiac sensing unit incorporating the portion of the one or more sensors, the cardiac sensing unit configured to be removably mounted onto the removable adhesive patch.

48. A non-transitory computer-readable medium for assessing future cardiac risk for a patient, the computer-readable medium comprising program instructions that, when executed by at least one processor, cause the at least one processor to:
identify cardiovibration data points corresponding to a predetermined physiological marker of the patient based on a digitized cardiovibration signal provided by a wearable cardiac sensing device configured to be removably, bodily-attached to the patient, and configured to sense cardiovibration signals from a patient and provide the digitized cardiovibration signal;
analyze a frequency spectrum corresponding to the identified cardiovibration data points to determine one or more cardiovibration frequency metrics for the patient associated with the predetermined physiological marker of the patient;
provide the one or more cardiovibration frequency metrics for the patient to a trained cardiovibration classifier, wherein the trained cardiovibration classifier is trained at least in part on historical cardiovibration frequency metrics associated with the predetermined physiological marker derived from a plurality of patients;
optimize the trained cardiovibration classifier based on one or more predetermined classifier evaluation metrics; and
output, based on the trained and optimized cardiovibration classifier, risk information concerning a future cardiac ischemia and/or a sudden cardiac arrest event occurring within a predetermined future period of time.

49. The non-transitory computer-readable medium of claim 48, wherein the predetermined physiological marker comprises a first heart sound (S1) corresponding to closure of the atrioventricular valve of the patient, and wherein the program instructions further cause the at least one processor to:
obtain the digitized cardiovibration signal;
identify heart beats within the obtained digitized cardiovibration signal by identifying local R-peaks within the obtained digitized cardiovibration signal; and
identify a set of points adjacent to the identified local R-peak corresponding to a S1 region for each identified heartbeat.

50. The non-transitory computer-readable medium of claim 48, wherein the predetermined physiological marker comprises a second heart sound (S2) corresponding to aortic closure and pulmonic closure for the patient, and wherein the program instructions further cause the at least one processor to:
obtain the digitized cardiovibration signal;
identify heart beats within the obtained digitized cardiovibration signal by identifying local R-peaks within the obtained digitized cardiovibration signal;
identify a corresponding T-wave for each identified local R-peak; and
identify a set of points adjacent to the identified T-wave corresponding to a S2 region for each identified heartbeat.

51. The non-transitory computer-readable of claim 48, wherein the analyzing the frequency spectrum corresponding to the identified cardiovibration data points to determine one or more cardiovibration frequency metrics for the patient associated with the predetermined physiological marker further comprises:
performing a fast fourier transform on the identified cardiovibration data points to determine a frequency spectrum for the identified cardiovibration data points, wherein the frequency spectrum decomposes the identified cardiovibration data points into components of different frequencies; and
determining a power spectral density for the determined frequency spectrum.

52. The non-transitory computer-readable medium of claim 48, wherein the program instructions further cause the at least one processor to at least one of: display the risk information on a display screen, generate a medical report comprising the risk information, initiate an alarm responsive to the risk information, or initiate a therapeutic protocol responsive to the risk information.

53. A non-transitory computer-readable medium for assessing future cardiac risk for a patient, the computer-readable medium comprising program instructions that, when executed by at least one processor, cause the at least one processor to:
identify ECG data points corresponding to premature ventricular contractions (PVCs) of the patient based on a digitized ECG signal, wherein the digitized ECG signal is provided by a wearable cardiac sensing device configured to sense electrocardiogram (ECG) signals from the patient and is configured to be removably, bodily-attached to the patient;
determine one or more PVC patterns based on the identified ECG data points corresponding to the PVCs of the patient;
determine one or more of a count or a burden associated with the one or more PVC patterns; and
output risk information concerning sudden cardiac arrest event occurring within a predetermined future period of time by applying a trained machine learning algorithm to the determined one or more of the count or the burden associated with the patient, wherein the trained machine learning algorithm is trained at least in part on historical PVC pattern data derived from a plurality of patients.

54. The non-transitory computer-readable medium of claim 53, wherein the one or more PVC patterns comprises a PVC run or an n-geminy, and a burden associated with the one or more PVC patterns comprises a count associated with the one or more PVC patterns over the total time of the digitized ECG signal, wherein the PVC run is indicative of the number of consecutive PVCs, and the PVC run is one of a singlet, couplet, triplet, quadruplet, or Non-sustained ventricular tachycardia (NSVT), and the PVC pattern comprises a n-geminy, wherein an n-geminy is indicative of the interval between PVCs, and the n-geminy is one of a bigeminy, trigeminy, quadrageminy, or 5-geminy.

55. The non-transitory computer-readable medium of claim 53, wherein the program instructions further cause the at least one processor to at least one of: display the risk information on a display screen, generate a medical report comprising the risk information, initiate an alarm responsive to the risk information, or initiate a therapeutic protocol responsive to the risk information.

56. A non-transitory computer-readable medium for assessing future cardiac risk for a patient, the computer-readable medium comprising program instructions that, when executed by at least one processor, cause the at least one processor to:
identifying ECG data points corresponding to premature ventricular contractions (PVCs) of the patient based on a digitized ECG signal provided by a wearable cardiac sensing device configured to be removably, bodily-attached to the patient, the wearable cardiac sensing device configured to sense electrocardiogram (ECG) signals from a patient and provide the digitized ECG signals;
determining multi-focal PVC data from the identified ECG data points corresponding to PVCs by applying an unsupervised machine learning algorithm, wherein the unsupervised machine learning algorithm is trained to determine multiple focal points within a set of PVCs; and
generating risk information for the patient indicative of a future sudden cardiac arrest event by applying a trained classifier to the determined multi-focal PVC data, wherein the classifier is trained on a historical data set of multi-focal PVC data derived from a plurality of patients.

57. The non-transitory computer-readable medium of claim 56, wherein the program instructions further cause the at least one processor to:
identifying QRS notch data points corresponding to QRS notches of the patient based on the digitized ECG signal;
wherein generating risk information for the patient comprises applying a second classifier trained on a historical data set of QRS notch data derived from a plurality of patients.

58. The non-transitory computer-readable medium of claim 56, wherein the program instructions further cause the at least one processor to at least one of: display the risk information on a display screen, generate a medical report comprising the risk information, initiate an alarm responsive to the risk information, or initiate a therapeutic protocol responsive to the risk information.
